(19) United States
(12) Patent Application Publication
(10) Pub. No.: US 2021/0339024 A1
(43) Pub. Date: Nov. 4, 2021

(54) THERAPEUTIC SPACE ASSESSMENT

(71) Applicant: ALPHA OMEGA NEURO TECHNOLOGIES LTD., Nof HaGalil (IL)

(72) Inventors: Omer NAOR, Kiryat-Tivon (IL); Hagai BERGMAN, Jerusalem (IL); Alaa HANNA, Sakhnin (IL); Sunbula MASALHA, Shefamar (IL); Nabeel SAKRAN, Nazareth (IL); Imad YOUNIS, Nazareth Ilit (IL); Goerge ASAD, Nazareth (IL); Salam AUKAL, Shefa-Amr (IL)

(73) Assignee: Alpha Omega Neuro Technologies Ltd., Nof HaGalil (IL)

(21) Appl. No.: 17/273,326

(22) PCT Filed: Sep. 6, 2019
(86) PCT No.: PCT/IB2019/057524

§ 371 Date: Mar. 4, 2021

Related U.S. Application Data
(60) Provisional application No. 62/727,641, filed on Sep. 6, 2018.

(51) Int. Cl. A61N 1/36; A61N 1/372; A61N 1/05

(52) U.S. Cl. CPC A61N 1/36067; A61N 1/36139; A61N 1/36185; A61N 1/0534; A61N 1/36171; A61N 1/37247

(57) ABSTRACT

A method for selecting stimulation treatment parameter values, including:

receiving signals related to a patient condition from at least one sensor, during and/or following at least one brain stimulation session, in which stimulation is delivered in at least one location within the brain, using at least one set of treatment parameter values;
analyzing the received signals to quantitatively assess at least one treatment side effect and at least one symptomatic effect;
selecting a set of treatment parameter values based on the quantitative assessment of the treatment side effects and the symptomatic effect.

### RELATED APPLICATION/S

This application claims the benefit of priority under 35 USC § 119(e) of U.S. Provisional Patent Application No. 62/727,641 filed 6 Sep. 2018, the contents of which are incorporated herein by reference in their entirety.

### FIELD AND BACKGROUND OF THE INVENTION

The present invention, in some embodiments thereof, relates to therapeutic space assessment and, more particularly, but not exclusively, to therapeutic space assessment of a brain stimulation treatment.

Movement disorders can be defined as neurological conditions that affect the speed, fluency, quality, and ease of movement, and may result from hereditary, acquired or idiopathic causes. In some movement disorders, such as Parkinson's Disease, there are present additional signs and symptoms that can be noted, and whose evaluation is important for the diagnosis as well as for the assessment of the severity of the disease.

The assessment of the movement disorder's signs and symptoms can be important in diagnosis of the disease, during the disease treatment and following the treatment.

The following are some attempts to assess movement disorders and their symptoms: “A novel assistive method for rigidity evaluation during deep brain stimulation surgery using acceleration sensors” by Ashesh Shah et al.,“A portable system for quantitative assessment of arkinsonian rigidity” by Houde Dai et al., “A Novel Method for Systematic Analysis of Rigidity in Parkinson's Disease” by Takayuki Endo et al., “Measurement of Rigidity in Parkinson's Disease” by Arthur Prochazka et al., “Quantification of Hand Motor Symptoms in Parkinson's Disease: A Proof-of-Principle Study Using Inertial and Force Sensors” by JOSIEN C. VAN DEN NOORT et al., “Research and Development of a Portable Device to Quantify Muscle Tone in Patients with Parkinsons Disease” by David Wright et al., “QAPD: An Integrated System to Quantify Symptoms of Parkinson's Disease” by Vrajeshri Patel et al., “Assessing bradykinesia in Parkinson's disease using gyroscope signals” by S. Summa et al., “An Adaptive Model Approach for Quantitative Wrist Rigidity Evaluation during Deep Brain Stimulation Surgery” by Sofia Assis et al., and “A Mobile Cloud-Based Parkinson's Disease Assessment System for Home-Based Monitoring” by Di Pan et al.

Additional background art includes U.S. Pat. Nos. 9,289,603 and 9,282,928.

### SUMMARY OF THE INVENTION

Some examples of some embodiments of the invention are listed below:

Example 1. A method for selecting stimulation treatment parameter values, comprising:
receiving signals related to a patient condition from at least one sensor, during and/or following at least one brain stimulation session, in which stimulation is delivered in at least one location within the brain, using at least one set of treatment parameter values;
analyzing said received signals to quantitatively assess at least one treatment side effect and at least one symptomatic effect;
selecting a set of treatment parameter values based on said quantitative assessment of said treatment side effects and said symptomatic effect.
Example 2. A method according to example 1, comprising mapping a therapeutic space based on results of said quantitative assessment of said at least one treatment side effect and said at least one symptomatic effect.
Example 3. A method according to any one of examples 1 or 2, wherein said analyzing comprises analyzing said received signals following an implantation surgery.
Example 4. A method according to any one of examples 1 to 3, wherein said analyzing comprises analyzing said received signals during an implantation surgery.
Example 5. A method according to any one of the previous examples, wherein said analyzing comprises analyzing said received signals using one or more statistical methods to quantitatively assess said at least one treatment side effect and at least one symptomatic effect.
Example 6. A method according to any one of the previous examples comprising recording said received signals when the patient is at rest and/or when a patient performs a task.
Example 7. A method according to example 1, wherein said selecting comprises selecting said set of treatment parameter values based on future flexibility of said selected set of treatment parameter values.
Example 8. A method according to example 7, comprising calculating a range of said future flexibility, and wherein said selecting comprises selecting said set of treatment parameter values based on said calculated range.
Example 9. A method according to example 8, comprising delivering an indication regarding said future flexibility value.
Example 10. A method according to example 9, wherein said future flexibility is based on a tuning ability of said stimulation treatment when selecting a set of treatment parameter values.
Example 11. A method according to any one of examples 7 to 10, wherein said future flexibility is based on future changes in an at least one therapeutic effect modifier capable of affecting therapy and/or tuning ability in the future.
Example 12. A method according to example 11, wherein said therapeutic effect modifier comprises one or more of disease progression, drug regime, future changes in treatment side effects, and/or future changes in disease symptoms.
Example 13. A method according to any one of examples 11 or 12, wherein said therapeutic effect modifier comprises future changes in stimulation location and/or future changes in electrode configuration.
Example 14. A method according to any one of examples 11 to 13 comprising scoring said at least one therapeutic effect modifier, and wherein said delivering comprises delivering a visual indication regarding said scoring.
Example 15. A method according to any one of examples 9 to 14, wherein said delivering comprises delivering a visual indication regarding a group of treatment parameter value sets, and wherein said selecting comprises selecting said set of treatment parameter values from said group.
Example 16. A method according to example 7, comprising calculating a desired future flexibility value prior to said selecting, and mapping a therapeutic space based on said desired future flexibility value and said treatment parameter values set used for brain stimulation.
Example 17. A method according to example 16, comprising delivering a visual indication regarding said therapeutic space, and wherein said selecting comprises selecting said set of treatment parameter values based on said visual indication.
Example 18. A method according to any one of the previous examples, comprising determining that at least one stimulation electrode and/or an electrode lead is in a selected position inside the brain based on said quantitative assessment of said treatment side effects and said symptomatic effect.
Example 19. A method according to any one of the previous examples, wherein said analyzing comprises analyzing said received signals to quantitatively assess one or more of gaze deviation and diplopia, continuous activation of muscles in legs, arms or face, dyskinesia, muscle rigidity, tremor and bradykinesia.
Example 20. A method according to any one of the previous examples, wherein said selecting comprises selecting a set of values related to stimulation amplitude, stimulation frequency and/or stimulation duration of a stimulation treatment.
Example 21. A method according to any one of the previous examples wherein said brain stimulation comprises deep brain stimulation.
Example 22. A method for mapping therapeutic space, comprising:
receiving signals related to a patient condition from at least one sensor, during and/or following at least one brain stimulation delivered in at least one location within the brain, using at least one set of treatment parameter values;
analyzing said received signals to quantitatively assess at least one treatment side effect and at least one symptomatic effect;
mapping therapeutic space based on said quantitative assessment.
Example 23. A method according to example 22, wherein said mapping comprises mapping said therapeutic space based on a desired future flexibility.
Example 24. A method according to any one of examples 22 or 23, comprising recording said signals when the patient is at rest and when a patient performs a task.
Example 25. A method according to any one of examples 22 to 24, comprising determining that a stimulation electrode or an electrode lead is positioned in a correct location inside the brain.
Example 26. A method according to any one of examples 22 to 25, comprising selecting at least one set of treatment parameter values based on said mapping of said therapeutic space.
Example 27. A method according to any one of examples 22 to 26 comprising delivering an indication regarding said therapeutic space.
Example 28. A system for selecting a set of treatment parameter values for a brain stimulation treatment, comprising:
a control circuitry;
a memory connected to said control circuitry, wherein said memory stores signals related to a patient condition measured during and/or following at least one brain stimulation, at least one set of treatment parameter values used for said brain stimulation;
an analysis circuitry connected to said control circuitry, wherein said control circuitry signals said analysis circuitry to quantitatively assess at least one treatment side effect and at least one symptomatic effect of said brain stimulation based on said stored signals;
a user interface connected to said control circuitry, wherein said user interface is configured to deliver an indication regarding said at least one treatment side effect and said at least one symptomatic effect.
Example 29. A system according to example 28, wherein said control circuitry generates a map of a therapeutic space based on said quantitative assessment of said at least one treatment side effect and at least one symptomatic effect, and signals said user interface to deliver an indication regarding said mapped therapeutic space.
Example 30. A system according to example 29, wherein said control circuitry calculates at least one optional set of treatment parameter values based on said mapped therapeutic space.
Example 31. A system according to example 30, wherein said control circuitry signals said user interface to deliver an indication related to said at least one optional set of treatment parameter values.
Example 32. A system according to any one of examples 29 to 31, wherein said control circuitry calculates a relation between at least one set of treatment parameter values and said mapped therapeutic space, and signals said user interface to deliver an indication regarding said relation.
Example 33. A system according to any one of examples 29 to 32, wherein said control circuitry maps the therapeutic space based on at least one desired future flexibility range or score stored in said memory.
Example 34. A system according to example 28, wherein said analysis circuitry calculates said at least one value of a future flexibility based on said quantitative assessment of said at least one treatment side effect and said at least one symptomatic effect and/or said at least one set of treatment parameter values stored in said memory.
Example 35. A system according to example 34, wherein said analysis circuitry calculates said at least one value of said future flexibility based on a future effect of at least one therapeutic effect modifier comprising disease progression, future changes in treatment side effects, future changes in disease symptoms, future changes in stimulation location, future changes in number and/or combination of stimulation electrodes, and drug regime.
Example 36. A system according to example 35, wherein said user interface is configured to generate a graphical representation of a level of future effect of said at least one therapeutic effect modifier.
Example 37. A system according to example 35, comprising a communication circuitry connected to a remote database, and wherein said communication circuitry receives said at least one value related to future flexibility from said remote database.
Example 38. A system according to example 35, wherein said at least one value related to future flexibility is calculated based on a large dataset collected from a plurality of patients.
Example 39. A system according to any one of examples 28 to 38 wherein said user interface is configured to display a list of treatment parameter values sets suitable for a delivery of brain stimulation, based on said at least one treatment side effect and said at least one symptomatic effect.
Example 40. A system according to example 34, wherein said analysis circuitry is configured to generate a therapeutic space based on said at least one future flexibility value, said quantitative assessment of aid at least one side effect and said at least one treatment side effect, and said at least one set of treatment parameter values used for said at least one stimulation.
Example 41. A system according to example 40, wherein said user interface is configured to display a graphical representation of said generated therapeutic space around said at least one set of treatment parameter values used for said at least one stimulation.
Example 42. A system according to example 35, wherein said analysis circuitry generates a score for each of a plurality of therapeutic effect modifiers, and wherein said user interface is configured to display a graphical representation of said generated scores with relation to said therapeutic effect modifiers.
Example 43. A system according to any one of examples 28 to 42, wherein said at least one treatment side effect comprises gaze deviation, diplopia, continuous activation of muscles in legs, arms or face, and dyskinesia.
Example 44. A system according to any one of examples 28 to 43, wherein said at least one symptomatic effect comprises one or more of muscle rigidity, tremor and bradykinesia.
Example 45. A system according to example 30, wherein said control circuitry is connected to a programmer of a DBS system, and wherein said control circuitry is configured to directly program said DBS system using said programmer based on an input received from a user via the user interface.
Example 46. A method for detection of DBS-induced gaze disorder, comprising: receiving baseline signals recorded at eye movements prior to brain stimulation, and stimulation-related signals recorded at eye movement during brain stimulation;
identifying segments in the signals indicative of eye movements;
calculating a value of a change in the signal level in the identified segments;
comparing changes in values between the baseline signals and stimulation-related signals;
detecting stimulation-induced gaze disorder in said stimulation-related signals based on said comparison.
Example 47. A method for quantification of rigidity, comprising:
receiving baseline signals and stimulation-induced signals from at least one EMG electrode placed on a body of a patient, while said patient is at rest;
measuring in said signals an average signal feature localized around at least one selected time point;
calculating at least one central tendency parameter of said measured average signal feature in said stimulation-induced signals to detect changes in the stimulation-induced signal compared to said baseline signals;
quantifying a reduction in rigidity based on said detected changes.
Example 48. A method according to example 47, comprising:
identifying a reduction in a power of a frequency band in a range of 20-2000 Hz, and wherein said quantifying comprising quantifying a reduction in rigidity based on said identified reduction.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electromagnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A is a flow chart of a general process for programming a brain stimulation system, for example a DBS system, according to some embodiments of the invention;

FIG. 1B is a flow chart of a detailed process for programming a brain stimulation system, for example a DBS system, according to some embodiments of the invention;

FIG. 1C is a flow chart of a general process for assessment of a current condition future considerations when selecting treatment parameter values, according to some embodiments of the invention;

FIG. 1D is a flow chart of a process for selection or treatment parameter values, according to some embodiments of the invention;

FIG. 1E is a schematic illustration of a therapeutic space, according to some exemplary embodiments of the invention;

FIG. 2A is a block diagram of a system for assessment of a patient condition and selection of treatment parameter values, according to some embodiments of the invention;

FIG. 2B is a schematic illustration of a processing method of therapeutic effect modifiers and optimization certainty, according to some embodiments of the invention;

FIG. 3 is a block diagram of a system for assessment of a patient condition, according to some embodiments of the invention;

FIG. 4A is a flow chart of a general process for quantification of a patient condition following task performance, according to some embodiments of the invention;

FIG. 4B is a flow chart of a process for quantification of a patient condition using statistical inference and/or machine learning methods, according to some embodiments of the invention;

FIG. 5 is a flow chart of a process for quantification of neurological disease symptoms and/or treatment side effects, according to some embodiments of the invention;

FIG. 6A is a flow chart of a process for pulse generator programming based on quantitative assessment of patient symptoms and treatment side effects, according to some embodiments of the invention;

FIG. 6B is a flow chart of a process for pulse generator programming based on quantitative assessment of patient symptoms and treatment side effects and prior data from previous assessments, for example data from large dataset and/or operating room electrophysiology, according to some embodiments of the invention;

FIG. 7A is a flow chart of a process for generation of at least one index, according to some embodiments of the invention;

FIG. 7B is a flow chart of a process for generation of at least one index following separation of tremor and non-tremor related signals, according to some embodiments of the invention;

FIG. 7C is a flow chart of a process for task-related index calculations compared to a baseline, according to some embodiments of the invention;

FIGS. 8A-8C are flow charts of different methods for separation of tremor-related signals from non-tremor related signals, according to some embodiments of the invention;

FIGS. 9A-9C is a panel of graphs showing the application of an absolute value are flow charts of different processes of a signal for identification of tremor, used in an experiment and according to some embodiments of the invention;

FIG. 9D is a graphical representation of the results of the processes described in FIGS. 9A-9C, according to some embodiments of the invention;

FIGS. 9E-9G are tables showing correlation between analysis results and manual assessment, as performed in the experimental analysis;

FIG. 9H is a graph showing a high pass filter having a 1 Hz cutoff, used in an experiment and according to some embodiments of the invention;

FIGS. 10A and 10B are schematic illustrations showing locations for placing EMG electrodes, as used in an experiment and according to some embodiments of the invention;

FIGS. 11A-11F and 12 are graphs showing different analysis stages of a signal received from EMG electrodes, as used in an experiment and according to some embodiments of the invention;

FIGS. 13A and 13B are panels of graphs showing results of a tremor analysis process and a rigidity analysis process, performed during an experiment and according to some embodiments of the invention;

FIG. 14A is a schematic illustration of locations on a face for placement of electrodes for gaze assessment, according to some embodiments of the invention;

FIGS. 14B-14I are graphs showing stages and results of a gaze analysis process, performed during an experiment and according to some embodiments of the invention;

FIG. 15A is a schematic illustration of locations on a face for placement of electrodes for assessment of internal capsular recruitment, as used in an experiment and according to some embodiments of the invention;

FIGS. 15B-15C are graphs showing identification of a signal segment indicative of motor movement, in an experiment and according to some embodiments of the invention; and

FIGS. 16A-16D are screen shots of a display of a software for assessment of patient condition, according to some embodiments of the invention.

### DESCRIPTION OF SPECIFIC EMBODIMENTS OF THE INVENTION

The present invention, in some embodiments thereof, relates to therapeutic space assessment and, more particularly, but not exclusively, to therapeutic space assessment of a brain stimulation treatment.

An aspect of some embodiments relates to programming a brain stimulation system, for example a DBS system, based on quantitative assessment of at least one side effect of the treatment and/or at least one symptomatic effect of the treatment. In some embodiments, the quantitative assessment of the at least on side effect and/or the at least one symptomatic effect is used to update unfinished programming performed during an implantation surgery, for example a surgery in an operating room, of at least one stimulation electrode or an electrode lead. In some embodiments, the quantitative assessment of at least one side effect of the treatment and/or at least one symptomatic effect of the treatment is used to update a therapeutic space map defined during the surgery. In some embodiments, the programming is performed outside the operating room.

According to some embodiments, an assessment system, for example a patient condition assessment system is used for the quantitative assessment of the at least one side effect and/or the at least one symptomatic effect. In some embodiments, the system provides a feedback to a person programming the DBS system, for example a human programmer, regarding one or more sets of treatment parameter values. In some embodiments, the feedback is generated and delivered to the human programmer based on the assessment of the at least side effect and/or the at least one symptomatic effect. In some embodiments, the feedback is generated and provided regarding the option to program the DBS system with a set of treatment parameter values selected by the human programmer. In some embodiments, treatment parameters comprise stimulation location, number of stimulation electrodes, location of stimulation electrodes, combination of stimulation electrodes, stimulation amplitude, stimulation frequency, stimulation pulse width and stimulation duration.

According to some embodiments, the assessment system generates and provides the feedback to the human programmer based on the performed quantitative assessment and/or information inserted manually to the system by a user of the system or the human programmer. Alternatively or additionally, the assessment system generates and delivers the feedback to the human programmer based on information from a large dataset collected from a plurality of patients.

An aspect of some embodiments relates to selecting treatment parameter values of a neurological treatment, for example a brain stimulation treatment, based on a desired future flexibility, for example a desired leeway, of the therapy. In some embodiments, the treatment parameter values are selected based on a desired future flexibility of a specific set of treatment parameter values, for example when delivering the stimulation at a selected location within the brain. In some embodiments, the desired future flexibility is quantified and the quantification result is used when selecting the treatment parameter values. In some embodiments, the quantification results indicate the level of flexibility needed to allow modification of the treatment in the future when selecting a specific set of treatment parameter values. In some embodiments, the treatment parameter values are selected based on the desired future flexibility and quantitative assessment of the patient condition, for example quantitative assessment of at least one side effect and/or at least symptomatic effect of the therapy.

According to some embodiments, the treatment parameter values are selected during an implantation surgery of at least one stimulation electrode or an electrode lead. In some embodiments, the selected treatment parameter values are used for programming of a stimulation system, for example a DBS system in the operating room. In some embodiments, feedback is delivered to a human programmer of the DBS system, for example a surgeon, regarding a potential of treatment parameter values selected by the human programmer to be used for programming. In some embodiments, the feedback is generated and delivered to the human programmer based on a comparison between future flexibility of the selected treatment parameter values and the desired future flexibility. In some embodiments, the feedback includes suggestions for one or more alternative treatment parameter values sets. In some embodiments, during an implantation surgery in an operating room, the at least one stimulation electrode or electrode lead is moved to a different location based on the delivered feedback. In some embodiments, the feedback is used

According to some embodiments, the desired future flexibility is based on estimated changes in the future of at least one therapeutic effect modifier capable of affecting the delivered therapy. Alternatively or additionally, the desired future flexibility is based on an optimization certainty that at least one stimulation electrode or an electrode lead are in a desired location within the brain, or a certainty to complete an optimization process of selection treatment parameter values in a predetermined time period, for example during a time of an implantation surgery. In some embodiments, the desired future flexibility is estimated for a time period of at least one day, at least one week, at least one month, at least one year, at least 10 years or any intermediate, shorter or longer time period, following a transplantation surgery of at least one electrode or an electrode lead in the brain of a patient, or in a different embodiment, following programming of a pulse generator, for example an implanted pulse generator (IPG).

According to some embodiments, the desired future flexibility is quantified based on measurements of an assessment system measuring the response and/or condition of a single patient to stimulation delivered using at least one treatment parameter values set. Alternatively, the desired future flexibility is quantified based on a large dataset. In some embodiments, the large dataset is generated by collection of data from a plurality of patients that contains information regarding the effect of one or more therapeutic effect modifiers on a stimulation therapy during different time periods following an implantation surgery and/or following reprogramming of an IPG. In some embodiments, one or more of at least one algorithm, at least one statistical method, at least one lookup table is applied on the large dataset to generate a value, for example a score, for the potential effect of one or more therapeutic effect modifiers on outcomes of a stimulation therapy. In some embodiments, the value is generated for the potential effect of one or more therapeutic effect modifiers on a therapy delivered using one or more of a specific set of treatment parameter values, a specific stimulation location, a specific number and/or combination of stimulation electrodes delivering the stimulation. In some embodiments, a user inserts information related to the desired future flexibility manually to an assessment device.

According to some embodiments, patient measurement features are matched with previous data. In some embodiments, the matching is used to predict how patient act. In some embodiments, the prediction is based on patients having similar anatomy, progression and/or stimulation devices.

According to some exemplary embodiments, the quantification results of the desired future flexibility are presented to a user, for example an expert. In some embodiments, the quantification results are presented, for example on a display, in relation to one or more of a specific set of treatment parameter values, for example in relation to a selected combination of values of a first treatment parameter, for example stimulation amplitude, and a values of a second treatment parameter, for example frequency. In some embodiments, additional treatment parameters comprise stimulation duration, number of stimulation pulses, stimulation location, location of at least one electrode used for stimulation, number of electrodes used for stimulation, and a specific combination of electrodes used for the stimulation.

According to some embodiments, a display to the user includes two or more sets of treatment parameter values, side effects and/or symptomatic effect of the two or more sets and/or therapeutic space, for example shape and/or size of the therapeutic space.

According to some embodiments, the information received from the assessment system, for example the quantification of the patient condition, the mapping of the therapeutic space and/or calculating a desired future flexibility is used to determine whether the electrode or electrode lead is positioned in a desired stimulation location and/or that a selected configuration of electrodes is a desired configuration.

According to some exemplary embodiments, two or more stimulations are delivered to the brain, each with a different set of stimulation parameter values. In some embodiments, per each set of stimulation parameter values a desired future flexibility value, for example a score, is calculated.

In some embodiments, the future flexibility value is a range of values in at least one treatment parameter, for example a range of the intensity of the stimulation, a range of stimulation frequency values. In some embodiments, the score indicates the level of flexibility needed to allow modification of the treatment in the future, when selecting a set from the at least two different stimulation parameter values sets or a different potential set of treatment parameter values estimated from at least one set used for stimulation. In some embodiments, the calculated score is used, for example to rank potential treatment parameter values sets. In some embodiments, the ranking, the calculated score for each treatment parameter values set are presented, for example on a display, to a user.

According to some embodiments, the at least one therapeutic effect modifier comprises current disease symptoms and/or estimated changes in disease symptoms in the future. Alternatively or additionally, the at least one therapeutic effect modifier comprises a current drug regime of the patient and/or estimated changes in the drug regime of the patient in the future, for example due to age or clinical condition in the future. Alternatively or additionally, the at least one therapeutic effect modifier comprises a healing process from the implantation surgery. In some embodiments, during the healing process, changes in the tissue surrounding the stimulation electrode or tissue placed in contact with the stimulation electrode change the response of the tissue to delivered treatment, change the effect of the treatment on disease symptoms and/or change the appearance of side effects.

According to some embodiments, the at least one therapeutic effect modifier comprises a stimulation location, for example estimated changes in the stimulation location, use of a different stimulation electrode or a different combination of stimulation electrodes in the future. Alternatively or additionally, the at least one therapeutic effect modifier comprises disease progression, for example progression of the disease or a specific type of he disease in the future. In some embodiments, progression of the disease optionally leads to a need to deliver a more robust treatment, for example by changing treatment parameter values. Alternatively or additionally, the at least one therapeutic effect modifier comprises stimulation parameter values, for example estimated changes in stimulation parameter values in the future. Alternatively or additionally, the at least one therapeutic effect modifier comprises treatment side effects, for example estimated changes in the treatment side effect in the future. Optionally, the treatment side effects are side effects of a combination between one or more drugs administered to the patient and the stimulation treatment.

According to some embodiments, a future flexibility level, for example a value or score, is updated, for example while a stimulation electrode is implanted in the brain of a patient and/or therapy is delivered. In some embodiments, the future flexibility level is updated based on measurements of the patient condition, for example measurements of at least one symptomatic effect and/or at least one treatment side effect, performed while the patient is at his home or at a clinic. In some embodiments, the future flexibility level is updated based on changes in at least one therapeutic effect modifier or changes in a score of said at least one therapeutic effect modifier. In some embodiments, the future flexibility level is updated based on information received from analysis of a large dataset.

According to some exemplary embodiments, an indication, for example an alert signal, is delivered to the patient and/or to an expert or a person monitoring the condition of the patient, for example if the updated future flexibility level is not a desired future flexibility level. In some embodiments, the patient and/or the expert stops the stimulation treatment and/or programs the stimulation system with a different set of treatment parameter values. In some embodiments, the alert signal is delivered if the updated future flexibility level is smaller than a pre-determined value. In some embodiments, the indication or the updated future flexibility level is transmitted to the expert or a person monitoring the patient condition by wireless transmission or other tele-medicine methods.

An aspect of some embodiments relates to mapping a therapeutic space, for example a therapeutic window (TW) of a stimulation treatment, for example a brain stimulation treatment, based on a desired future flexibility of a therapy. In some embodiments, the defined therapeutic space includes at least one set of treatment parameter values that lead to a desired therapeutic effect on the patient, and has a desired future flexibility that allows changing of the treatment parameter values in the future while maintaining the desired therapeutic effect, and optionally maintaining a desired levels of side effects.

According to some embodiments, the therapeutic space is mapped based on a quantitative assessment of treatment side effects and symptomatic effect during and/or following stimulation and/or based on quantification of a desired future flexibility. In some embodiments, the term stimulation session refers to a session in which one or more stimulation pulses are actively delivered to a tissue. In some embodiments, the term “during stimulation” refers to during at least one stimulation session. In some embodiments, the term “following stimulation”, refers to following at least one stimulation session, when stimulation is not actively delivered to the tissue. In some embodiments, the therapeutic space is defined per a specific stimulation location and/or per a specific combination of two or more stimulation electrodes used to deliver a stimulation treatment. In some embodiments, the therapeutic space is mapped per a fixed location of at least one stimulation electrode or an electrode lead, taking into account that the electrode or lead cannot be moved after surgery.

According to some embodiments, the therapeutic space is displayed to a user, for example by a graphical representation of the therapeutic space. In some embodiments, the therapeutic space includes two or more regions that differ based on a symptomatic effect level, a side effects level and/or future flexibility. In some embodiments, the two or more regions are generated by clustering treatment parameter values sets that generate a symptomatic effect and/or lead to side effects within a pre-determined range of values. In some embodiments, the two or more regions are generated by clustering treatment parameter values that have a future flexibility level within a pre-determined range of values. In some embodiments, the two or more regions are scored, for example based on the level of one or more of the symptomatic effect level, the side effects level or a future flexibility level. In some embodiments, the graphical representation of the therapeutic space includes a graphical representation of the two or more regions, and/or a score of the groups.

According to some embodiments, the therapeutic space is updated based on future changes one or more therapeutic effect modifiers. Alternatively or additionally, the therapeutic space is updated based on future assessments of the patient condition, for example assessment of symptomatic effect and/or treatment side effect. In some embodiments, the updated therapeutic space is stored in a memory of an assessment device. In some embodiments, an indication, for example an alert signal is delivered to a patient or a person monitoring the patient condition, for example by wireless transmission or other tele-medicine methods, if the updated therapeutic space is not a desired therapeutic space. In some embodiments, the alert signal is delivered, if the updated therapeutic space size is reduced below a predetermined value.

According to some embodiments, the assessment system and methods described herein are used in an operating room. In some embodiments, in the operating room, a human programmer, for example a user of the system determines which stimulation to perform and where in the brain. In some embodiments, the assessment system provides the human programmer a map of the therapeutic space, for example in a form of a graphical representation of the therapeutic space. In some embodiments, the human programmer verifies a set of selected treatment parameter values using the map. Alternatively or additionally, the human programmer selects at least one set of alternative parameter values based on the information in the map, for example a set of treatment parameter values included in the map. In some embodiments, the assessment system and methods are used in the operating room to make sure that the implanted stimulation electrode or electrode lead are positioned in a correct place prior to completing the operating procedure. In some embodiments, the stimulation is performed from an acute navigating electrode or from one or more contacts of an implanted electrode lead.

According to some embodiments, the assessment system and methods described herein are used in an IPG programming session, for example a programming session performed outside the operating room, for example in a clinic. In some embodiments, in the programming session, the assessment results are used to select an optimal set of treatment parameters for chronic therapy. According to some embodiments, during the programming session, the assessment system provides suggested treatment parameter values sets to the user, or parameters that would lead to an optimally efficient search of the DBS parameters, that is most likely to end satisfactorily in a minimum time.

An aspect of some embodiments relates to using Electrooculography (EOG) to quantify stimulation induced gaze disorder side effect. In some embodiments, the effect of brain stimulation on the stimulation-induced gaze is quantified by comparing eye-movement related signals recorded prior to stimulation to eye-movement related signals recorded during and/or following stimulation. In some embodiments, segments in the recorded signals indicative of eye movements are identified. In some embodiments, a value related to a change in the signal in the segments is calculated. In some embodiments, the stimulation-induced gaze disorder is identified and quantified by comparing calculated change related values between signals recorded prior to stimulation, and the signals recorded during and/or following stimulation.

An aspect of some embodiments relates to quantification of rigidity based on signals measured before and after brain stimulation. In some embodiments, the signals are measured by at least one electrode, for example an EMG electrode connected to the patient. In some embodiments, rigidity is quantitatively assessed in the operating room, for example during an implantation surgery. In some embodiments, rigidity is quantified by measuring an average signal feature localized around at least one selected time point in the signals. Alternatively or additionally, rigidity is quantified by calculating at least one calculating at least one central tendency parameter of the averaged signal. In some embodiments, rigidity is quantified by identifying a reduction in a power of a frequency band in a range of 20-2000, for example 20-500 Hz, 200-1000 Hz, 1000-2000 Hz 1500-2000 Hz or any intermediate, smaller or larger frequency range, in stimulation-induced signals, for example signals recoded during a stimulation session.

A potential advantage of receiving a feedback from an assessment system as described herein, during an implantation process in an operating room is that the at least one stimulation electrode or electrode lead can be moved to a different location inside the brain. A potential advantage of receiving a feedback from the assessment system outside the operating room is that there is more time to perform fine tuning of the treatment parameter values, for example to reach an optimal therapeutic effect.

According to some embodiments, the methods and systems described below are used outside an operating room, for example in a clinic.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary General Programming Process

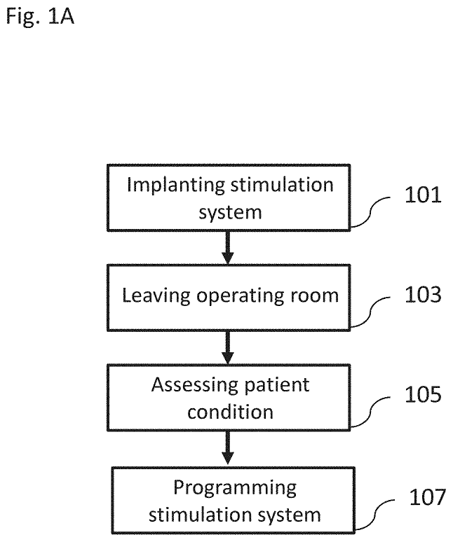

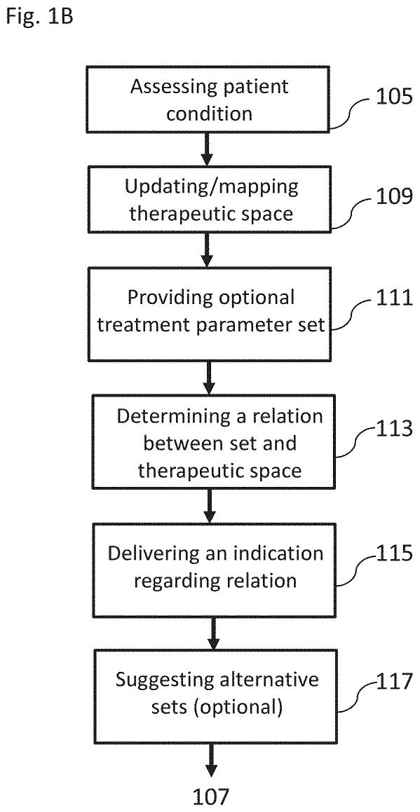

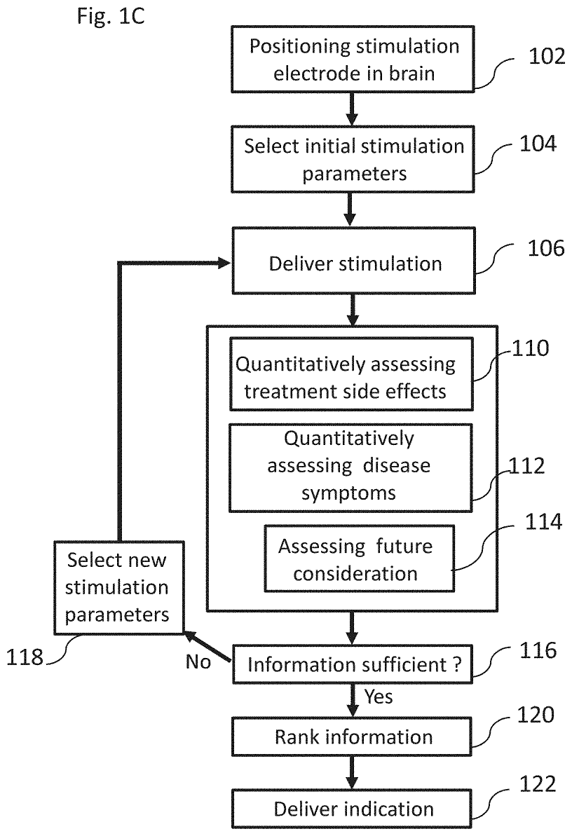

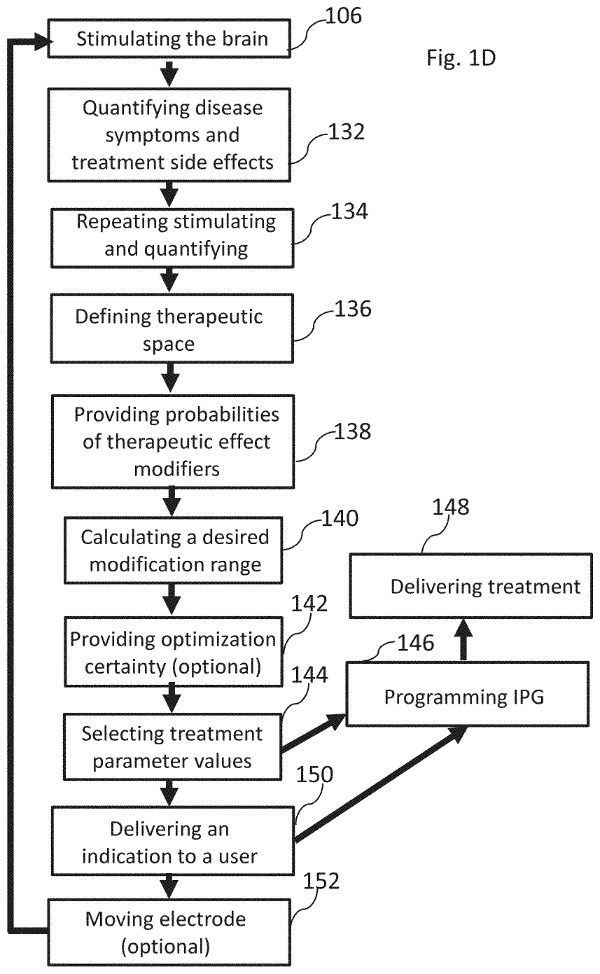

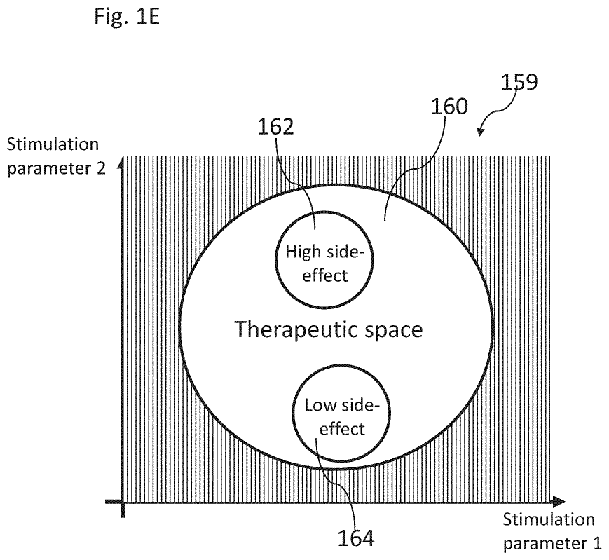

According to some exemplary embodiments, the assessment system and methods described herein are used for programming a brain stimulation system, for example a pulse generator of a brain stimulation system outside operating room. In some embodiments, the programming is performed after a healing process from an implantation surgery. Reference is now made to FIG. 1A, depicting a general programming process following an implantation procedure, according to some exemplary embodiments of the invention.

According to some exemplary embodiments, a stimulation system, for example at least one stimulation electrode or an electrode lead is implanted in a brain of a patient at block 101. In some embodiments, the stimulation system is implanted in an operating room, during an implantation surgery. In some embodiments, in the operating room the stimulation system is programmed with an unfinished program.

According to some exemplary embodiments, the patient leaves the operating room at block 103.

According to some exemplary embodiments, patient condition is assessed at block 105. According to some exemplary embodiments, the patient condition, for example at least one treatment side effect and/or at least one symptomatic effect is quantitatively assessed at block 105. In some embodiments, the patient condition is assessed during a programming session, for example programming session performed at the home of the patient or at a clinic. In some embodiments, the patient condition is assessed during a recovery period from the implantation surgery or following the recovery period.

According to some exemplary embodiments, the stimulation system, for example a pulse generator of the stimulation system, is programmed at block 107. In some embodiments, the stimulation system is programmed in a programming session, performed at the home of the patient or at the clinic. In some embodiments, the stimulation system is programmed based on the results of the patient condition assessment. Additionally, the stimulation system is programmed based on a desired future flexibility. In some embodiments, the stimulation system is programmed based on information received from a human programmer performing the programming and/or information received from a remote computer or a remote server. In some embodiments, the programming comprises updating an operating room unfinished program.

### Exemplary Detailed Programming Process

Reference is now made to FIG. 1B, depicting a detailed programming process, according to some exemplary embodiments of the invention.

According to some exemplary embodiments, patient condition is assessed at block 105, for example as described in FIG. 1A.

According to some exemplary embodiments, a therapeutic space is mapped at block 109. Alternatively, an existing therapeutic space, for example a therapeutic space mapped during an implantation surgery is updated at block 109. In some embodiments, the therapeutic space is mapped or updated based on the assessment of the patient condition. Additionally, the therapeutic space is mapped or updated based on desired future flexibility.

According to some exemplary embodiments, at least one optional treatment parameter values set is provided to the assessment system, at block 111. In some embodiments, the at least one optional treatment parameter values set is provided by a user, for example a human programmer, during a programming session.

According to some exemplary embodiments, a relation between the provided set and the therapeutic space is determined at block 113. In some embodiments, the assessment system determines whether the provided set is included or not included within the therapeutic space. In some embodiments, the assessment system determines a distance between the provided set to the margins of the therapeutic space.

According to some exemplary embodiments, an indication regarding the determined relation is delivered at block 115. In some embodiments, the assessment system delivers the indication to the user, for example the human programmer. In some embodiments, the assessment system delivers the indication as a feedback to the user, about the ability to program the stimulation system using the provided set.

According to some exemplary embodiments, alternative treatment parameter values sets are suggested at block 117. In some embodiments, the assessment system suggests alternative sets to the user, for example based on input from the user, for example the provided treatment parameter set. In some embodiments, the assessment system suggests the alternative sets based on the therapeutic space. Additionally, the assessment system suggests the alternative sets based on a desired future flexibility. In some embodiments, the system suggests the alternative sets, based on information received from the user, from the patient and/or from a large data set collected from a plurality of patients.

According to some exemplary embodiments, the user, for example the human programmer selects a set of treatment parameter values, for example from the lists of suggested sets, for programming the stimulation system at block 107. In some embodiments, the user selects a set for programming based on the indication delivered at block 115. In some embodiments, the user selects a set based on information displayed by the system regarding the therapeutic space and/or desired future flexibility.

### Exemplary General Process for Quantifying Expected Therapeutic Effect Modifiers

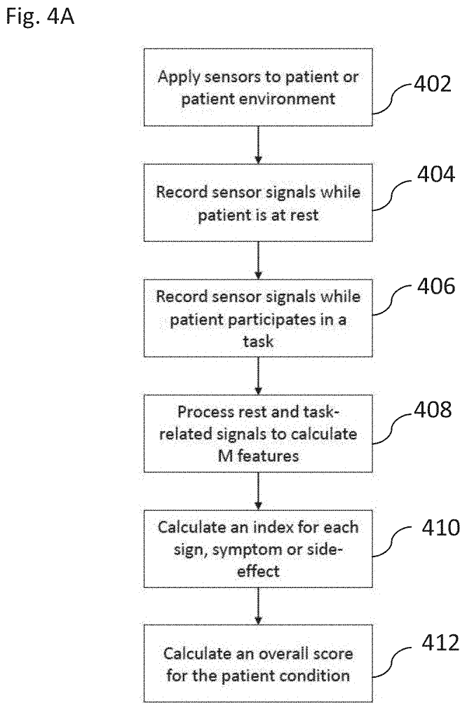

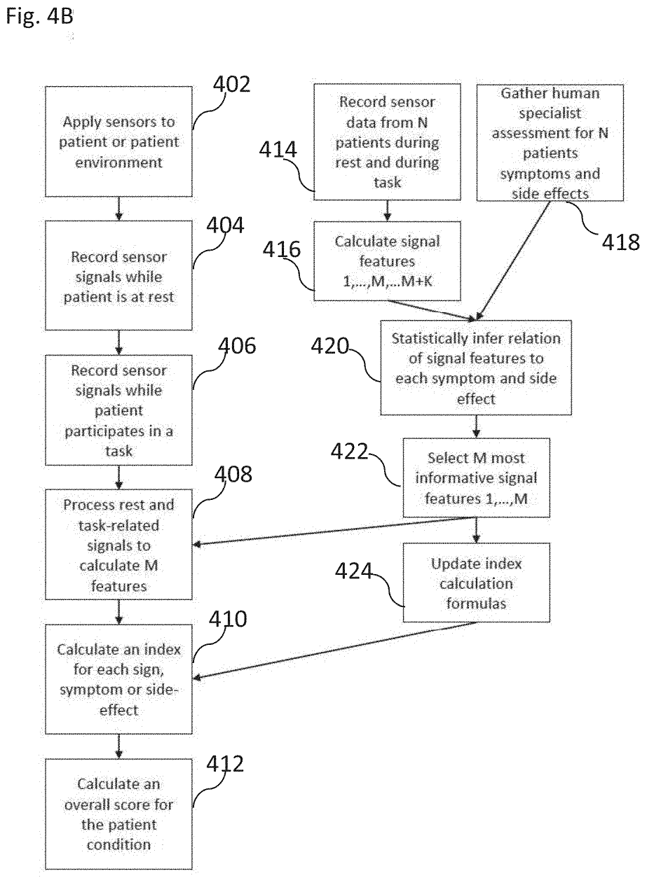

According to some exemplary embodiments, therapeutic effect modifiers are quantified using machine leaning/statistical methods, for example as describe in FIG. 4B. In some embodiments, expert labeled data, in which a neurologist is assessing the patient over time, is combined with an assessment of the patient condition over time in IPG programing and at home with the home-system edition. In some embodiments, the received data is labeled by the expert or the system. In some embodiments, pre-operation and intra-operation data (for example as described below) is used in one of the methods described for example in FIG. 4B, for example to get a most accurate prediction of the changes measured post-op.

According to some exemplary embodiments, therapeutic effect modifiers are quantified based on a large dataset collected from a plurality of patients. In some embodiments, the large dataset is generated by collection of data prior to the surgery, for example data related to one or more of disease stage and duration, severity of symptoms using the assessment system described herein or clinical assessment, severity of medication side effects using the system described herein or a clinical assessment, medication regime history, familial diseases, genetic indications, imaging data and/or mobile phone data or data collected by different sensors, for example GPS data which optionally relates to how much the patient walks, accelerometer data optionally related to small-scale movements of the patient, and/or microphone optionally related to quality of articulation.

According to some exemplary embodiments, data is collected during a DBS surgery, for example one or more of general medical and demographic data, MER data, stimulation quantification data, video data, audio data, data related to decision, for example decisions where to implant the stimulation electrode or lead.

According to some exemplary embodiments, data is collected following an implantation surgery, for example an updated information regarding the therapeutic space using different treatment parameter values sets, data from patients in their home environment, who at least sporadically use the assessment system for assessment of their symptoms/side effect. In some embodiments, this system includes an input device such as a tablet, in which the patients perform additional tasks, input their personal self-assessments, or play games and/or participate in other interactive activities, for example activities that include providing input to the device, that also quantify their motor condition. In some embodiments, the data comprises mobile phone data or data received from other sensors, for example GPS data accelerometer data; data from medical records, data collected from visits to neurologist, data related to changes in medication and/or imaging data.

According to some exemplary embodiments, the pre-surgery and intra-operation data is used to predict how the therapy will be modified in the future. In some embodiments, a large dataset infrastructure is used, that can store many tera bytes and possibly peta-bytes of data, and can apply computational algorithms to the large set of data, for example unlabeled data to extract information. In some embodiments, the applied algorithms include algorithms to extract the most important information from medical records, as described e,g, in J. Jiang “Information extraction from text”, C.C. Aggarwal, C. Zhai (Eds.), Mining text data, Springer, United States (2012), pp. 11-41. Also include audio analytics techniques, extracting information that is indicative of the patients condition. Some of it may rely on how we quantify dysarthria in our system, some of it may be as described in J. Hirschberg, A. Hjalmarsson, N. Elhadad “You're as sick as you sound”: Using computational approaches for modeling speaker state to gauge illness and recovery A. Neustein (Ed.), Advances in speech recognition, Springer, United States (2010), pp. 305-322.

In some embodiments, processing the data comprises extracting meaningful information from the video of the patient, for example recorded by the assessment system pre-operation, intra-operation or post-operation, optionally n combination of one or more indexing techniques, for example indexing techniques described in W. Hu, N. Xie, L. Li, X. Zeng, S. Maybank. A survey on visual content-based video indexing and retrieval IEEE Transactions on Systems, Man, and Cybernetics, Part C: Applications and Reviews, 41 (6) (2011), pp. 797-819.

According to some exemplary embodiments, methods to combine the information extracted from the various sources, and use it to find the relation between the patient's condition before and during the surgery to how the condition would vary in the future, are performed using one or more methods described in J. Fan, F. Han, H. Liu “Challenges of big data analysis”. National Science Review, 1 (2) (2014), pp. 293-314, or in J. Fan, J. Lv, “Sure independence screening for ultrahigh dimensional feature space” Journal of the Royal Statistical Society: Series B (Statistical Methodology), 70 (5) (2008), pp. 849-911.

### Exemplary General Process for Selecting Treatment Parameters

According to some exemplary embodiments, parameter values of a stimulation treatment, for example a DBS treatment, are selected based on a current status of a disease, system and patient, and future needs for therapy of the patient. Reference is now made to FIG. 1C, depicting a general process for selecting stimulation parameters, according to some exemplary embodiments of the invention.

According to some exemplary embodiments, at least one stimulation electrode is positioned within the brain. In some embodiments, the at least one stimulation electrode is located on an electrode lead, for example an electrode lead shaped as a needle, inserted into the brain. In some embodiments, the at least one stimulation electrode is part of a plurality of electrodes axially and/or circumferentially displaced on the external surface of the lead. In some embodiments, the at least one stimulation electrode is placed in contact with brain tissue. In some embodiments, the at least one stimulation electrode and/or electrode lead is positioned at a predetermined location within the brain, for example at a desired anatomical and/or functional location.

According to some exemplary embodiments, at least one stimulation electrode is positioned within the brain at block 102. In some embodiments, the at least one stimulation electrode is placed inside the brain in an implantation procedure. In some embodiments, the at least one stimulation electrode is located on an electrode lead, for example an electrode lead shaped as a needle, introduced into the brain.

According to some exemplary embodiments, initial stimulation parameter values are selected at block 104. In some embodiments, the initial stimulation parameter values are selected based on the position of the stimulation electrode within the brain. Additionally, or alternatively, the stimulation parameter values are selected according to safety considerations. Optionally, the initial stimulation parameter values are selected based on knowledge from a large dataset which includes data collected from a plurality of patients. In some embodiments, stimulation parameters comprise one or more of stimulation amplitude, stimulation frequency, stimulation duration, number of stimulation pulses in a train of pulses, the duration of each individual pulse, or pulse-width, number of trains, overall number of stimulation pulses in a time period, for example per minute, per hour, per day.

According to some exemplary embodiments, stimulation is delivered through the at least one stimulation electrode at block 106. In some embodiments, the stimulation is delivered according to the selected initial stimulation parameter values.

According to some exemplary embodiments, a quantitative assessment of the treatment side effects is performed at block 110. In some embodiments, the treatment side effects comprise gaze deviation and diplopia, unclear articulation of speech (dysarthria), continuous activation (recruitment) of muscles in legs, arms or face, and unintentional movement (dyskinesia). In some embodiments, the quantitative assessment is performed in a timed relationship with the delivery of stimulation, for example during and/or following the delivery of stimulation. In some embodiments, the quantitative assessment is performed in a time period of up to 30 minutes, for example up to 10 minutes, up to 5 minutes, up to 1 minute, up to 30 seconds or any intermediate, shorter or longer time period from the end of stimulation. Alternatively or additionally, the quantitative assessment is performed at least 1 second from the beginning of stimulation, for example 1 second, 10 seconds, 30 seconds, 1 minute, 10 minutes or any intermediate, shorter or longer time period from the beginning of stimulation.

According to some exemplary embodiments, a quantitative assessment of the disease symptoms is performed at block 112. In some embodiments, the disease symptoms comprise muscle rigidity (resistance to passive movement of a limb), tremor and bradykinesia defined as slowness or lack of movement. In some embodiments, the quantitative assessment is performed in a timed relationship with the delivery of stimulation, for example before, during and/or following the delivery of stimulation. In some embodiments, the quantitative assessment is performed in a time period of up to 30 minutes, for example up to 10 minutes, up to 5 minutes, up to 1 minute, up to 30 seconds or any intermediate, shorter or longer time period from the end of stimulation. Alternatively or additionally, the quantitative assessment is performed at least 1 second from the beginning of stimulation, for example 1 second, 10 seconds, 30 seconds, 1 minute, 10 minutes or any intermediate, shorter or longer time period from the beginning of stimulation.

According to some exemplary embodiments, future considerations related to the stimulation treatment are assessed, for example to calculate a desired future flexibility of the therapy, at block 114. In some embodiments, the desired future flexibility is based on estimated changes in the future, of at least one therapeutic effect modifier capable of affecting the delivered therapy. In some embodiments, at least one therapeutic effect modifier include the healing process, for example the healing process of the tissue surrounding the at least one stimulation probe, disease progression, changes in drug regime over time, possible need to change in stimulation location, changes in disease symptoms and treatment side effects over time, and/or possible need to change stimulation parameter over time. In some embodiments, the desired future flexibility is calculated to allow, for example, unfinished programming in the operating room with sufficient future flexibility to allow tuning of the programming following the implantation surgery, for example following or during a recovery period of the patient from the surgery.

According to some exemplary embodiments, the overall information provided to a user or to a system is assessed at block 116. In some embodiments, if the information provided is not sufficient to allow selection of treatment parameter values, then new stimulation parameters are selected at block 118, instead of the initial treatment parameter values, and the assessment process is repeated by delivering a stimulation at block 106 using the new stimulation parameter values.

Optionally, at least one stimulation electrode or an electrode lead is moved to a different location.

According to some exemplary embodiment, if the provided information is sufficient, then the information is ranked at block 120. In some embodiments, the information is ranked using one or more statistical methods and/or algorithms, for example machine learning algorithms. In some embodiments, the information is ranked, for example to generate one or more recommendations to a user of the device, for example to an expert.

According to some exemplary embodiments, an indication is delivered to the expert at block 122. In some embodiments, the indication is a human detectable indication, optionally provided on a display. In some embodiments, the indication is a graphical indication showing a ranking of one or more options according to a selected scoring system.

### Exemplary Detailed Process for Selection of Treatment Parameters

Reference is now made to FIG. 1D depicting detailed process for selection of treatment parameters based on current status and future consideration, according to some exemplary embodiments of the invention.

According to some exemplary embodiments, stimulation is delivered at block 106, for example as described in FIG. 1C. According to some exemplary embodiments, disease symptoms and/or treatment side effects are quantified at block 132, for example as described in FIG. 1C.

According to some exemplary embodiments, stimulation is repeated at block 134. In some embodiments, stimulation is repeated at least one more time, for example 2, 3, 5, 10 times or any intermediate, smaller or larger number of times. In some embodiments, the stimulation is repeated each time with different treatment parameter values.

According to some exemplary embodiments, a therapeutic space is defined at block 136. In some embodiments, a therapeutic space, for example a multi-dimensional space is defined by two or more treatment parameter values that promote a therapeutic effect. In some embodiments, the therapeutic space is defined based on the treatment parameter values used for the stimulation and the quantification of disease symptoms and treatment side effect following or during the stimulation.

According to some exemplary embodiments, information regarding therapeutic effect modifiers, capable of changing the therapeutic effect on the patient in the future, is provided at block 138. In some embodiments, therapeutic effect modifiers comprise changes in disease symptoms over time, changes in drug regime over time, healing process, possible changes in stimulation location, disease progression, changes in stimulation parameter values over time, changes in treatment side-effect over time. In some embodiments, the information is provided as statistical information, for example an index or a score, indicating the potential of a specific therapeutic effect modifier to affect the therapeutic effect in the future per selected stimulation parameter values.

According to some exemplary embodiments, a desired future flexibility, for example a desired modification range, is calculated at block 140. In some embodiments, the desired modification range is based on the information regarding the therapeutic effect modifiers, the therapeutic space and selected treatment parameter values. In some embodiments, the desired modification range, is a calculated or an estimated range in which the selected treatment parameter values will have to be changed in view of the effect of the therapeutic effect modifiers, in order to maintain the provided stimulation treatment within the defined therapeutic effect.

According to some exemplary embodiments, an optimization certainty is provided at block 142. In some embodiments, an optimization certainty refers to the level of certainty to complete an optimization process of treatment parameter values while the patient is in the operating room, when starting from the selected treatment parameter values, which optionally represent a point in the therapeutic space, and based on the desired modification range, the therapeutic space and the disease modifiers. In some embodiments, an optimization certainty is calculated or estimated based on the desired modification range, the therapeutic space for selected treatment parameter values.

According to some exemplary embodiments, treatment parameters values are selected at block 144. In some embodiments, at least one set, for example at least 2, 4, 10 sets or any intermediate, larger or smaller number of sets of treatment parameter values are selected at block 144. In some embodiments, a set of treatment parameter values comprises values for different treatment parameters. In some embodiments, the selected treatment parameter values are selected based on the current defined therapeutic space, the calculated desired modification range which refers to future events, and optionally on the optimization certainty.

According to some exemplary embodiments, the at least one selected set of treatment parameter values is used for automatic reprograming of an implanted pulse generator (IPG), at block 146. In some embodiments, once programming is completed, treatment is delivered at block 148.

Alternatively, and according to some exemplary embodiments, an indication, for example a human detectable indication, is delivered to the user at block 150. In some embodiments, the indication is a graphical representation. In some embodiments, the indication includes information regarding the selected at least one set of treatment parameter values.

According to some exemplary embodiments, the user moves the at least one stimulation electrode to a different location within the brain, at block 152, for example if the selected treatment parameter values are not the desired treatment parameter values. In some embodiments, the user moves the electrode lead on which the at least one stimulation electrode is positioned to a different location within the brain.

According to some exemplary embodiments, the user manually programs the IPG based on the selected treatment parameter values.

### Exemplary Therapeutic Space

Reference is now made to FIG. 1E, depicting a therapeutic space, according to some exemplary embodiments of the invention.

According to some exemplary embodiments, a multi-dimensional space 159 is defined in a coordinate system of two or more stimulation treatment parameters, for example stimulation parameter 1, for example stimulation amplitude, and stimulation parameter 2, for example stimulation frequency, shown in FIG. 1E. In some embodiments, each point within the space represents a different set of treatment parameter values of the two or more treatment parameters constructing the coordinate system. In some embodiments, a therapeutic space, for example therapeutic space 160 is a space included in the space 159 in which the sets of treatment parameter values comprised within the therapeutic space lead to a therapeutic effect, for example a desired therapeutic effect. In some embodiments, a therapeutic space is personalized for a patient or a group of patients. In some embodiments, the therapeutic space is generated, for example, by providing two or more stimulations using different sets of treatment parameter values and assessing disease symptoms and side effects during or following each stimulation event. Alternatively or additionally, the therapeutic space is generated, for example, by providing two or more stimulations using different stimulation electrodes or different combinations of stimulation electrodes.

According to some exemplary embodiments, the therapeutic space 160 includes one or more regions in which stimulation with the selected treatment parameters lead to side effects with varying levels, for example region 162 which includes stimulation parameter values that lead to a desired therapeutic effect with high level side effects, and region 164 which includes stimulation parameter values that lead to a desired therapeutic effect with low level side effects.

According to some exemplary embodiments, the one or more regions include treatment parameter value sets clustered based on similarity in therapeutic effect levels, side effects levels, or a calculated level of future similarity. In some embodiments, the similarity is based on a predetermined range or a predetermined threshold.

According to some exemplary embodiments, the therapeutic space, for example a size and/or shape of the therapeutic space, is determined based on the quantitative assessment of at least one symptomatic effect, at least one side effect and quantification of a desired future flexibility. In some embodiments, the therapeutic space, for example the therapeutic space size and/or shape, is updated as long as the patient continues to receive the stimulation therapy. In some embodiments, the therapeutic space is updated based on measurement of at least one side effect and/or at least one symptomatic effect performed following an implantation surgery, following programming of the IPG, for example while the patient is at home or at a clinic. In some embodiments, at least one indication, for example an alert signal, regarding the updated therapeutic space is delivered to the patient or to a person that monitors the patient condition.

According to some exemplary embodiments, the alert signal is delivered to the patient or the person monitoring the patient condition, if the updated therapeutic space, for example a size and/or the shape of the therapeutic space is not a desired therapeutic space. In some embodiments, the alert signal is transmitted to a remote device, for example a remote computer or cellular device.

### Exemplary System for Assessment of Patient Condition and Selection of Treatment Parameters

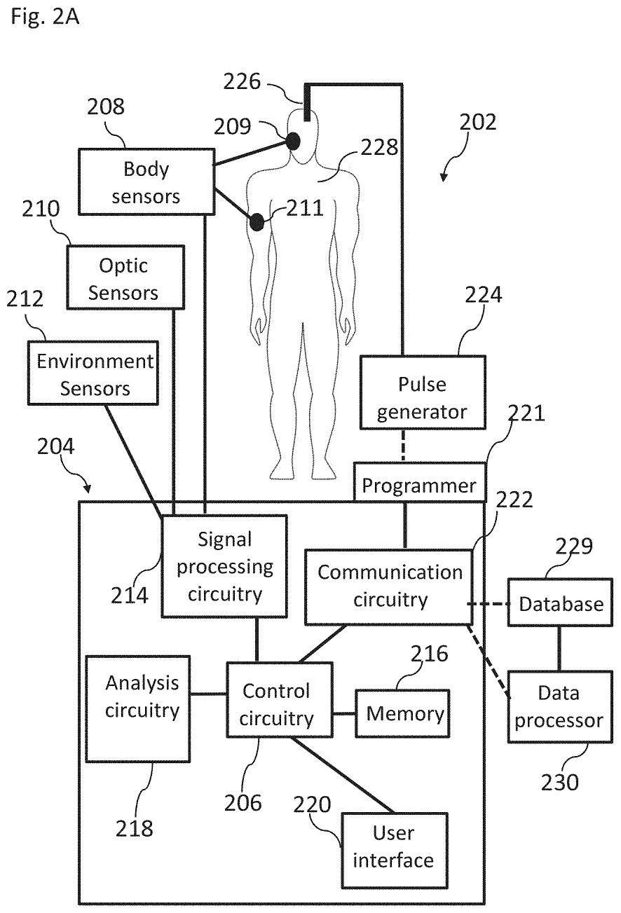

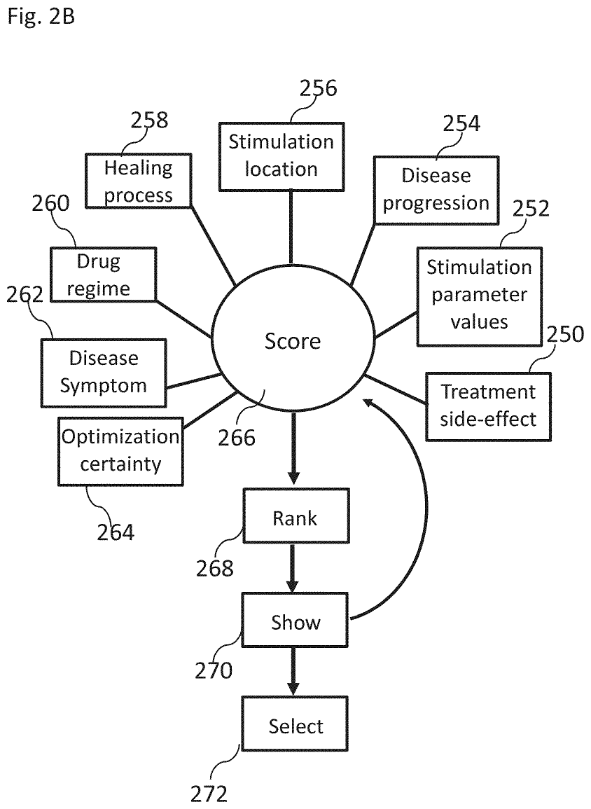

According to some exemplary embodiments, a system for assessment of a patient condition and selection of treatment parameters is used to assess the patient condition before, during and/or after the delivery of a brain stimulation treatment, for example a DBS treatment. In some embodiments, the assessment system is in direct communication with a DBS system, for example with an implanted pulse generator (IPG) of the DBS system, for example to automatically modify the DBS treatment or parameter values thereof. Alternatively, or additionally, the assessment system is in communication with a subject receiving the DBS treatment and/or with an expert, for example a physician, a technician or a nurse. In some embodiments, the subject and/or the expert modify the DBS treatment or parameter values thereof based on an indication received from the assessment system. Reference is now made to FIG. 2A, depicting a system for assessment of a subject condition, according to some exemplary embodiments of the invention.

According to some exemplary embodiments, a system for assessment of a subject condition comprises an assessment device, for example device 204 and one or more sensor connectable to the device 204. In some embodiments, the device 204 is a portable assessment device, shaped and sized to be attached to the body of a subject receiving the treatment, for example to the clothes of the subject, by at least one clip, hook, strap or any other attachment piece. In some embodiments, a weight of the assessment device is in a range of 100-500 grams. In some embodiments, the assessment device comprises a laptop, a tablet or a cellular device. Alternatively, the assessment device is a tabletop device, shaped and sized to be positioned on a table or a movable cart.

According to some exemplary embodiments, the system is constructed from one or more lightweight (up to 100 g) sensor modules attached to the patient's body. In some embodiments, the sensor modules transmit wireless signals to an external module. In some embodiments, the external module is not attached to the patient's body and is located, optionally, in the vicinity of the patient, for example in the patient's home, car, or a backpack or other carriable bag. In some embodiments, the required signal processing, analysis and subsequent communication occurs in the external module. In some embodiments, the external module serves as a communication relay, from which the data is transmitted to a remote cloud-based platform, and the signal processing and analysis is performed in the remote platform.

According to some exemplary embodiments, an initial stage of signal processing occurs in the nearby external module, that allows, for example to compress the data before transmission to the remote platform, thus reducing the required bandwidth for transmitting the data. In some embodiments, this compression may be achieved for example by averaging multiple repetitions of signals acquired in the same or similar condition, or by applying a transform that allows to reduce the amount of data required. For example, it is possible to perform a fast Fourier Transform (FFT), or a Discrete Cosine Transform (DCT), or other similar transforms and to discard data in frequency bands that is not required for subsequent signal processing and analysis. In some embodiments, the data is compressed by downsampling the signals, that is reducing the sample rate, allowing to discard some of the data. In some embodiments, a specific signal processing is performed on data sampled at a higher rate, for example estimating a shape of a high frequency transient, or estimating the frequency or magnitude of a high frequency component in the frequency domain. In this example and in some embodiments, the initial processing can be carried out on the nearby external module on signals acquired with the original, higher sample rate, followed by downsampling of the signal and transmission of the data in the lower sample rate to the remote platform, thus compressing the transmitted data and reducing bandwidth requirements.

According to some exemplary embodiments, the device 204 is configured to measure level of symptoms and/or changes in symptom levels of a neurological disease or a neurological condition, for example Depression, PD, essential tremor, Dystonia, Epilepsy, Obsessive-compulsive disorder, Addiction, Chronic pain, Cluster headache, Dementia, Huntington's disease, multiple sclerosis, Stroke, Tourette syndrome, and Traumatic brain injury. In some embodiments, the device 204 is configured to measure the symptom levels and/or changes in the symptom levels, based on signals received from the one or more sensor connectable to the device 204. Additionally, or alternatively, the device 204 is configured to measure side effect levels or changes in side effect levels of the brain stimulation treatment based on signals received from the one or more sensor. In some embodiments, some of the side effects comprise one or more of gaze deviation and diplopia, unclear articulation of speech (dysarthria) or poor speech volume control, continuous activation (recruitment) of muscles in legs, arms or face, unintentional movement (dyskinesia), impaired balance, for example due to problems with the functionality of the vestibular apparatus Paresthesia (abnormal skin sensation such as a tingling, pricking, chilling, burning, or numb sensation), Acute emotional response, for example acute mania or depression, impaired Impulse Control, Change in Heart Rate, Change in Blood pressure, Nausea/vomiting and Phosphenes (perception of light flashes).

According to some exemplary embodiments, the one or more sensor connectable to the device 204 comprises at least one body sensor 208, configured to be attached to the body of the subject, for example to allow sensing directly from the body of the subject. In some embodiments, the one or more body sensor comprises an EMG sensor, a magnetometer, an accelerometer, a gyroscope, a heartbeat sensor, hemoglobin oxygenation saturation sensor, blood pressure sensor, ECG sensor, EEG sensor, neuro-muscular transmission sensor, electro-dermal activity (or skin conductivity) sensor, respiratory monitor, thermometer. In some embodiments, the one or more body sensor is configured to be positioned on a head of the subject 228, for example on the face 209 of the subject. Alternatively, or additionally, the one or more body sensor is configured to be positioned on the body of the subject 228, for example on a limb 211 of the subject 228. Optionally, the body sensor is placed on a sticker or comprises a sticker, adhesively attachable to the body of the subject.

According to some exemplary embodiments, the one or more sensor connectable to the device 204 comprises at least one optic sensor, for example a video camera. In some embodiments, the optic sensor is configured to sense posture and/or movement of the body of the subject.

According to some exemplary embodiments, the one or more sensor connectable to the device 204 comprises at least one environment sensor configured to sense the environment or changes in the environment surrounding the subject, for example an audio sensor configured to capture a speech of a subject.

According to some exemplary embodiments, the one or more sensor is electrically connected to the device 204 via a signal processing circuitry, for example signal processing circuitry 214. In some embodiments, the signal processing circuitry 214 is electrically connected to a control circuitry 206 of the device 204. Additionally, the device 204 comprises a memory, for example a memory 216, electrically connected to the control circuitry 206. In some embodiments, the signal processing circuitry 214 is configured to process the signal from the one or more sensor, according to at least one signal processing algorithm and/or signal processing method stored in the memory 216. In some embodiments, for example when the signal received from the at least one sensor is an analog signal, the signal processing circuitry 214 is used to convert the analog signal into a digital signal. Additionally, or alternatively, the signal processing circuitry is configured to amplify the signals received from the one or more sensor. Additionally or alternatively, the signal processing circuitry is configured to assess and/or indicate the measurement quality, for example by measuring the impedance between an electrode and the patient tissue. Additionally or alternatively, the signal processing circuitry is configured to assess and/or indicate the quality of signals acquired over time, for example to detect signals with high amplitude transients which are related to external noise that may corrupt the measurement, or to calculate a signal-to-noise measure. Optionally the signal processing circuitry can reject low-quality signals from being fed as input to the signal processing chain that leads to assessing the patient condition. Additionally or alternatively, the signal processing circuitry is configured to estimate the current activity of the subject, for example, resting, walking, speaking, performing one of several predefined tasks required for the patient condition assessment, etc. Optionally, the estimate of the current subject activity is used to determine which subsequent signal processing and analyzing chains should be employed to the acquired signal. Alternatively and optionally, the estimate of the current subject activity is fed as additional input to subsequent signal processing chains, such that the signal processing chains employ different signal processing parameters or methods based on the current subject activity. In some embodiments, the signals received from the at least one sensor or indications thereof are stored in the memory 216. Additionally, or alternatively, the processed signals or indications thereof are stored in the memory 216.

According to some exemplary embodiments, the device 204 comprises an analysis circuitry, for example analysis circuitry 218, electrically connected to the control circuitry 206. In some embodiments, the analysis circuitry is configured to analyze the stored processed signals or indications thereof, for example to measure at least one side effect of the treatment and/or at least one disease symptom. In some embodiments, the analysis circuitry is configured to analyze the stored processed signals using at least one algorithm, for example a machine learning algorithm, stored in the memory 216. In some embodiments, based on the analysis of the stored processed signals, the analysis circuitry 218 calculates a score for each measured side effect of the treatment or an overall side effects score. Alternatively, or additionally, based on the analysis of the stored processed signals, the analysis circuitry 218 calculates a score for each measured symptom of the neurological disease or neurological condition, or an overall symptoms score.

According to some exemplary embodiments, the analysis circuitry 218 generates a quantitative assessment of the subject condition, for example as a subject condition score, based on one or both of the calculated side effects score and the calculated symptoms score. In some embodiments, the analysis circuitry 218 generates the subject condition quantitative assessment using at least one algorithm, for example a machine learning algorithm stored in the memory 216. In some embodiments, the calculated side effects score, the calculated symptom score, and/or the subject condition quantitative assessment are stored in the memory 216.

According to some exemplary embodiments, when the device 204 is in communication with a DBS system or with an IPG, the memory 216 stores log files of the DBS system or the IPG. Alternatively, or additionally, the memory 216 stores at least one DBS protocol or parameter values thereof. In some embodiments, the analysis circuitry is configured to determine a Therapeutic Space of the DBS treatment based on at least some of the stored log files, stored DBS protocol and/or stored parameter values of the DBS protocol.

According to some exemplary embodiments, the device 204 comprises a user interface 220, configured to generate and deliver at least one indication to the subject receiving the treatment and/or to an expert, for example a physician or a nurse. In some embodiments, the user interface 220 comprises a display and/or a speaker. In some embodiments, the indication is related to one or more of the calculated side effects score, the calculated symptoms score, the quantitative assessment of the subject condition and/or the determined TW. In some embodiments, the indication is a human detectable indication, for example an audio indication or a visual indication.

According to some exemplary embodiments, the indication, for example an alert signal, is delivered to the subject and/or to the expert if the quantitative subject condition assessment indicates that the subject condition is not within a desired TW. Alternatively, or additionally, the alert signal is delivered, for example when a modification of the DBS treatment, for example stopping the treatment or modifying one or more treatment parameter values is required.

According to some exemplary embodiments, the user interface 220 comprises one or more input interface, for example a button, a keyboard or any input interface configured to allow insertion of data into the device 204 and/or to activate at least one function of the device 204. In some embodiments, a subject receiving a DBS treatment uses the user interface 220 to activate the device 204 and to perform a quantitative assessment of the subject condition, following the subject feeling the side effects associated with the beginning of the treatment.

According to some exemplary embodiments, the device 204 comprises a communication circuitry 222, electrically connected to the control circuitry 206. In some embodiments, the communication circuitry 222 is configured to transmit and receive signals from a remote device, for example from a pulse generator 224 of a DBS system. In some embodiments, the pulse generator 224 delivers electrical pulses through an electrode lead, for example lead 226, to the brain of subject 228. In some embodiments, the communication circuitry is configured to receive and/or transmit wireless signals, for example Bluetooth, Wi-Fi, or any type of wireless signals to the DBS system, for example to the pulse generator 224.

According to some exemplary embodiments, the communication circuitry 222 is used as a programmer of the DBS system, for example as a programmer of the pulse generator 224. Alternatively, the assessment device 204 is connected to a programmer of a DBS system, for example programmer 221. In some embodiments, a user selects one or more suggested treatment parameter values sets, suggested by the device 204, and the device 204 transmits the information to the programmer 221 or to the pulse generator, for example via the communication circuitry 222. Alternatively, the device 204 displays one or more suggested treatment parameter values sets to a human programmer, and the human programmer manually programs the DBS system, for example via a programmer of the DBS system.

According to some exemplary embodiments, the device 204 receives wireless signals from the DBS system, for example from the pulse generator 224 of the DBS system, when electric pulses are delivered to the subject 228, when the delivery of pulses is initiated and/or when the delivery of pulses ends. In some embodiments, the control circuitry 206 signals the analysis circuitry to quantitatively assess the condition of the subject, in a time relationship, for example when the wireless signals from the pulse generator are received or in a selected time period following the receiving of the wireless signals, for example in a time period of up to 2 hours, up to 1 hour, up to 30 minutes, up to 10 minutes, up to 5 minutes, up to 1 minutes from receiving the wireless signals. In some embodiments, the control circuitry 206 signals the analysis circuitry 218 to quantitatively assess the condition of the subject in a time period of up to 2 hours, up to 1 hour, up to 30 minutes, up to 10 minutes, up to 5 minutes, up to 1 minutes or any intermediate, shorter or longer time period from receiving signals from the pulse generator 224 indicating that the delivery of electric pulses is finished.

According to some exemplary embodiments, the device 204 is configured to reprogram the pulse generator 224, for example when the delivered DBS treatment is not within a determined Therapeutic Space and/or when a quantitative assessment of the patient condition indicates an appearance of undesired side effects. In some embodiments, for example during the reprogramming, the control circuitry 206 signals the communication circuitry to transmit wireless signals to the pulse generator 224. In some embodiments, the transmitted signals include information regarding a new DBS protocol or a new set of DBS treatment parameter values selected to shift the effect of the treatment into the Therapeutic Space. In some embodiments, the transmitted signals include information regarding initiating or terminating DBS treatment based on changes in the assessment of patient condition.

According to some exemplary embodiments, the device 204 is in communication via the communication circuitry 222, with a database, for example a database including at least one data set. In some embodiments, the database 229 is stored on a server or in a cloud storage. In some embodiments, the database 229 stores information regarding results of stimulation of a patient with different stimulation parameters values. In some embodiments, the database 229 comprises information or indications regarding therapeutic effect modifiers, and the effect of therapeutic effect modifiers on a therapeutic effect of stimulation treatments delivered using the different stimulation parameters. In some embodiments, the database 229 includes information or indications regarding previously defined therapeutic spaces in different patients and/or optimization certainty in stimulation treatments of different patients.

According to some exemplary embodiments, the control circuitry 206 applies different statistical methods and/or algorithms on the large dataset stored in the database 229, for example generating scores and/or rankings of different treatment parameter values based on the large dataset. In some embodiments, the generated scores and/or rankings are presented to the user, for example to an expert using the user interface 220, for example on a display connected to the user interface.

According to some exemplary embodiments, an external data processor, for example data processor 230 applies different statistical methods on the large dataset stored in the database 229 for example to generate scores and/or rankings of different treatment parameter values based on the large dataset. In some embodiments, the device 204 receives the calculation results from the data processor, for example via the communication circuitry 222. In some embodiments, the calculation results, for example the scoring and ranking is delivered to the user by the user interface.

According to some exemplary embodiments, the user selects a set of treatment parameter values based on the provided scores and ranking, and the results of the patient condition. In some embodiments, the user uses the selected set of treatment parameter values to reprogram the pulse generator. Alternatively, the user decides to move the electrode lead 226 to a different location within the brain.

According to some exemplary embodiments, the control circuitry 206 is configured to quantify a desired future flexibility, for example a desired leeway. In some embodiments, the control circuitry 206 is configured to quantify the desired future flexibility per a specific treatment parameter values set and/or per a specific stimulation location. In some embodiments, the control circuitry 206 quantifies the desired future flexibility based on a specific set of treatment parameter values stored in the memory 216. Alternatively or additionally, the control circuitry 206 quantifies the desired future flexibility based on the patient condition assessment results.

According to some exemplary embodiments, the control circuitry 206 quantifies the desired future flexibility based on a value, for example a score, of at least one therapeutic effect modifier stored in memory 216. In some embodiments, the control circuitry 206 quantifies the desired future flexibility using at least one algorithm or a statistical method stored in the memory 216.

According to some exemplary embodiments, the control circuitry 206 quantifies the desired future flexibility based on a value, for example a score, of at least one therapeutic effect modifier stored in the database 229. In some embodiments, the score is received, for example from the data processor 230 via the communication circuitry 222. Alternatively, the score is received from a user via the user interface 220. In some embodiments, the control circuitry 206 is configured to generate a therapeutic space, based on the quantified future flexibility. In some embodiments, the control circuitry signals.

According to some exemplary embodiments, the control circuitry 206 signals the user interface 220 to deliver a visual indication, for example to display one or more of results of the quantification of the desired future flexibility, and/or the score of the at least one therapeutic effect modifier. Additionally or alternatively, the control circuitry 206 signals the user interface 220 to deliver a visual indication, for example to display, the generated therapeutic space.

According to some exemplary embodiments, the control circuitry 206 is configured to signal the user interface 220 to display one or more of the results of the desired future flexibility quantification, the score of the at least one therapeutic effect modifier and the therapeutic space by at least one graphical representation, for example a chart, a spider chart, a table, or a graph.

According to some exemplary embodiments, the control circuitry 206 is configured to calculate a score and/or rank for each set of at least two sets of treatment parameter values, based on quantification of future flexibility per each set of treatment parameter values. In some embodiments, the control circuitry 206 is configured to signal said user interface 220 to generate and deliver a visual indication, for example to display, the score and/or the ranking of the at least two sets of treatment parameter values.

According to some exemplary embodiments, the control circuitry 206 is configured to update an existing future flexibility and/or an existing therapeutic space stored in memory 216. In some embodiments, the control circuitry 206 updates the existing future flexibility and/or the existing therapeutic space based on at least one quantitative assessment of the patient condition performed, for example, when the patient is at home or at a clinic. Alternatively or additionally, the control circuitry 206 updates the existing future flexibility and/or the existing therapeutic space based on at least one indication, for example an indication related to at least one therapeutic effect modifier, received via the communication circuitry 222, for example from a remote computer or a remote server.

According to some exemplary embodiments, the control circuitry 206 signals the user interface 220 and/or the communication circuitry 222 to generate and deliver a human detectable indication, for example an alert signal, to the patient or to a person monitoring a condition of the patient, if the updated future flexibility is not a desired future flexibility, for example if the updated future flexibility indicates that a delivered therapy does not have a desired therapeutic effect and/or leads to undesired side effects. Alternatively or additionally, the control circuitry 206 signals the user interface 220 and/or the communication circuitry 222 to generate and deliver a human detectable indication, for example an alert signal, to the patient or to a person monitoring a condition of the patient, if the updated therapeutic space has a size and/or shape that a delivered therapy does not have a desired therapeutic effect and/or leads to undesired side effects.

According to some exemplary embodiments, the device 204 is a sensor box, for example an all-in-one sensor box, in which one or more sensors, for example the sensors described in FIG. 2A are connected or attached to the box.

### Exemplary Future Considerations of a Stimulation Treatment

Reference is now made to FIG. 2B, depicting different future considerations of a stimulation treatment and a general processing scheme, according to some exemplary embodiments of the invention.

According to some exemplary embodiments, different future considerations are addressed when selecting a set of treatment parameter values for a patient, for example to make sure that the delivered therapy remains efficient within a desired therapeutic space, in the future, for example in a month, in a year, in 10 years or any intermediate, shorter or longer time periods, after the implantation of the electrode.

According to some exemplary embodiments, the future considerations comprise at least one therapeutic effect modifier, having the potential to affect the therapeutic effect on a patient. In some embodiments, the at least one therapeutic effect modifier comprises a disease symptom 262, for example expected changes in the disease symptoms over time, that might affect the therapeutic effect of a treatment provided with parameter values determined at present, while the patient is in surgery.

According to some exemplary embodiments, the at least one therapeutic effect modifier comprises a drug regime 260, for example changes in the drug regime of the patient over time. In some embodiments, changes in the drug regime in the future can alter the response of the patient to the stimulation treatment.

According to some exemplary embodiments, the at least one therapeutic effect modifier comprises a healing process 258, for example the healing process of the brain tissue following the electrode lead implantation surgery. In some embodiments, the healing process may affect the tissue near the at least one stimulation electrode and optionally change the response of the tissue to the delivered stimulation.

According to some exemplary embodiments, the at least one therapeutic effect modifier comprises a stimulation location 256. In some embodiments, over time, stimulation location can be changed, for example to address other changes caused by one or more therapeutic effect modifiers. In some embodiments, changing the stimulation location can affect the therapeutic effect, for example reduce the therapeutic effect.

According to some exemplary embodiments, the at least one therapeutic effect modifier comprises disease progression 254. In some embodiments, the disease progression 254 is independent or dependent on the provided stimulation treatment. In some embodiments, the disease progression is changed due to the delivered stimulation treatment. In some embodiments, disease progression or changes in disease progression lead to optional changes in the treatment parameter values in the future.

According to some exemplary embodiments, the at least one therapeutic effect modifier comprises stimulation parameter values 252. In some embodiments, planned changes in stimulation parameters in the future, need to be addressed when selecting treatment parameter values at present.

According to some exemplary embodiments, the at least one therapeutic effect modifier comprises treatment side effects 250, for example changes in the treatment side effects over time. In some embodiments, known changes in the appearance of side effects in the future, need to be addressed when selecting treatment parameter values at present.

According to some exemplary embodiments, one of the future considerations is optimization certainty 264. In some embodiments, optimization certainty means the certainty to complete an optimization process of treatment parameter values selection in a limited time period of an implantation surgery, when starting the optimization process with a specific initial set of treatment parameter values.

According to some exemplary embodiments, the therapeutic effect modifiers and/or the optimization certainty are scored at block 266. In some embodiments, the therapeutic effect modifiers and/or the optimization certainty for specific treatment parameter values, for example treatment parameter values within the therapeutic space are scored. In some embodiments, each modifier is scored independently. Alternatively, a general score is calculated for all relevant therapeutic effect modifiers of a specific set of treatment parameter values. Optionally, a score for optimization circuitry is included in the general score for the specific set of treatment parameter values.

According to some exemplary embodiments, generated scores for different sets of treatment parameter values are ranked at bock 268. In some embodiments, the different sets of treatment parameter values are ranked according to the generated scores of each set.

According to some exemplary embodiments, the rankings and/or scores are presented to the user, for example an expert at block 270.

According to some exemplary embodiments, the user selects a specific set of treatment parameter values to reprogram the IPG at block 272. In some embodiments, the user selects the specific set based on the ranking and/or scores.

### Exemplary System for Quantitative Assessment of a Patient Condition

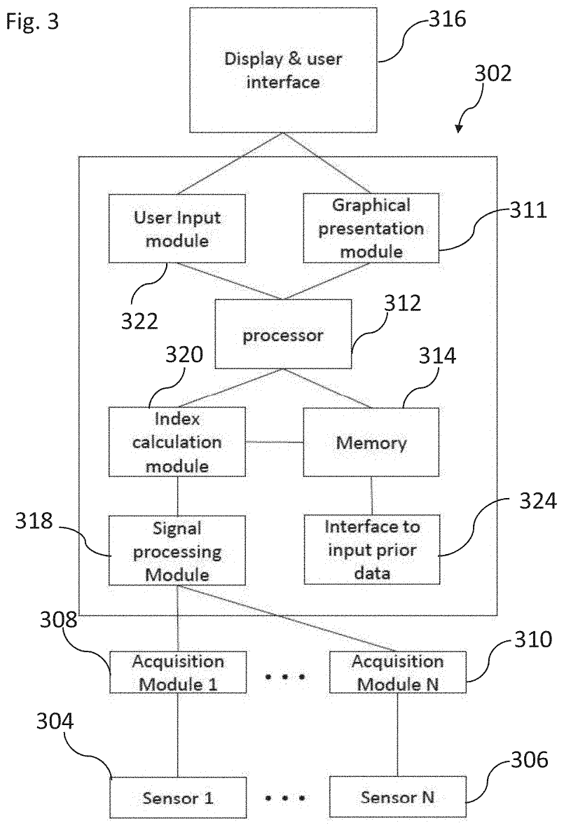

Reference is now made to FIG. 3 depicting a system for quantitative assessment of a patient condition, according to some exemplary embodiments of the invention.

According to some exemplary embodiments, a system for quantitative assessment of a patient condition, for example a system 302 includes one or more sensors, for example the sensors 304 and 306, and one or more acquisition modules, for example the acquisition modules 308 and 310, electrically connected to the sensors 304 and 306 respectively. In some embodiments, the communication and memory modules of the system, for example system 302, and the processing modules are used to carry out its function, for example to quantitatively assess the patient condition.

According to some exemplary embodiments, the sensors, for example sensors 304 and 306, are connected to the acquisition modules, for example acquisition modules 308 and 310 which perform initial signal conditioning on the analog and digitize it in an A2D. Additionally, the recorded data is transmitted to a processor, for example processor 312, which obtains instructions from a memory module(s), for example memory 314, as to how to perform the signal processing.

According to some exemplary embodiments, the system, for example system 302 also optionally includes one or more of video camera recordings, speech recordings, EMG signals recording, EO signals recordings, EEG recordings, position and/or orientation recordings, heartbeat recordings, hemoglobin oxygenation saturation recordings, blood pressure recordings, ECG recordings, neuromuscular transmission recordings, skin conductivity recordings, respiratory recordings, temperature recordings and or one or more of the gaze tracking devices detailed further below. Optionally the system also includes a display, to present the results to system operator, such as a subject, or clinician, as well as a user interface for user interaction with the system, for example display and user interface 316.

It should be noted that in some embodiments, different sensor types may contribute information that can be used to quantify the same attribute, for example a symptom, a clinical sign, a side of effect of a therapy whether pharmacological, electrical or other. In writing “sensor X is used to quantify attribute Y” it should be understood that in some embodiments the sensor X is used alone to quantify attribute Y, or it is used in conjunction with other sensors to quantify attribute Y in other embodiments. In the latter case, in some embodiments, the measures contributed from the various sensors are fused together into a single measure, for example via averaging—which could be simple or weighted averaging—or via a decision tree, or via another of the known methods to fuse various measures to a single measure.

According to some exemplary embodiments, the system is used to perform a step of normalization or standardization per each measure, for example to bring the various measures to a similar scale so they could be averaged in a meaningful way. For example, assumption is being made that an EMG-based measure of rigidity is found to typically vary between 20-5007, and the rigidity-sensing kinematic module typically produces values varying between 0-5 Nsec/m. Then optionally the first measure is normalized by subtraction of 20 followed by dividing by 30, to be brought to a 0-1 scale, while the second measure is divided by 5 also to be brought to a 0-1 scale, and then the two measures are averaged together.

Alternatively, the measures generated from the various sensors are fed as input to a statistical inference calculation, such as performed by a machine learning prediction algorithm, stored in the memory 314, which maps the set of input measures to a single output measure.

According to some exemplary embodiments, the system 302 comprises a signal processing module, for example signal processing module 318 electrically connected to one or more acquisition modules, for example acquisition module 308. In some embodiments, the signal processing module 318 is configured to process signals received from the sensors by one or more of filtering, envelope detection and spectral estimation (including mel-spectrum and cepstrum estimates), to detect peaks and calculate peak prominence values, to calculate various statistical measures on the signals, such as calculating an average, a standard deviation, a median, signal ranges and inter-quartile-ranges, to calculate correlations between signals from the same source or from different sources, to calculate cross-correlations between signals from the same source or from different sources, to align a signal to a trigger signal in time, to average two or more signals that are aligned in time or to subtract one signal from another, to detect high amplitude transient artifacts and optionally reject them, to perform impedance measurements and provide signal quality estimates. The main functions of the signal processing module are to verify input signal quality and to condition the signal to make it more suitable for analysis, for example by filtering out noises with a low-pass-filter or removing trends by high-pass-filters or by other methods. Further, the signal processing module applies techniques that highlight the signal features that are important to analysis, such as by applying transforms to frequency representations, or time-frequency representations in which spectral features or time-evolving spectral features are more easily estimated. Alternatively, the interesting features are highlighted by averaging two or more repetitions of the same type of signal, thus generally increasing the signal-to-noise ratio, or alternatively by subtracting one signal from another, or an ensemble of signals from another ensemble, thereby eliminating common-mode signal features and highlighting the differences between the signals. Before such subtraction or averaging, often alignment is required to ensure that delays in the acquisition times of the signals are removed, or at least taken into account. The end goal of the signal processing modules is to compute output numbers or signals, which can wither be presented to a user, or fed as input to an index calculation module that uses the input to provide an assessment index. In some embodiments, the signal processing module 318 is configured to process the signals received from the sensors, for example to obtain one or more signal feature, for example a value or a score calculated from a set of one or more signal input, and is used in the calculation of indices for the attributes of a subject.

According to some exemplary embodiments, the system 302 comprises an index calculation module, for example index calculation module 320 electrically connected to the processor 312. In some embodiments, the index calculation module 320 is configured to calculate an index, for example by combining the signal features obtained by the signal processing module, using one or more algorithms stored in the memory 314.

According to some exemplary embodiments, the system 302 comprises a user input module, for example user input module 322 electrically connected to the processor 312. In some embodiments, the user input module 322 comprises at least one button, a keypad, or a keyboard. In some embodiments, the user input module is configured to allow receiving signals and/or information from the user of the system 302.

According to some exemplary embodiments, the system 302 comprises an interface to input prior data 324 electrically connected to the memory 314, and configured to upload data from an external memory storage device into the memory 314. In some embodiments, the interface 324 comprises a flash drive interface, and/or a USB interface.

According to some exemplary embodiments, the system 302 comprises a graphical presentation module 311, electrically connected to the processor 312 and to the display and user interface 316. In some embodiments, the processor signals the graphical representation module to generate a graphical representation of a therapeutic space, scores or values of at least one therapeutic effect modifier and/or quantification results of future flexibility. In some embodiments, the graphical presentation module generates the graphical representation as described in FIG. 2A in relation to the user interface 220.

### Exemplary Methods for Quantitative Assessment of Patient Condition

According to some exemplary embodiments, a method for quantification of some movement disorders symptoms and side effects, for example DBS-induced side effects, using an array comprising at least one sensor of at least one type of sensors. In some embodiments, the sensors include, one or more of all of EMG electrode(s), EOG electrode(s), eye-tracking sensor(s), audio recorder(s), video camera(s) and a rigidity-sensing module(s) with at least one accelerometer, at least one gyroscope and/or at least one force meter.

According to some exemplary embodiments, the sensors are applied to the subject, or the subject environment (e.g. an audio recorder is placed in the vicinity of the subject, eye tracker is placed in a position enabling direct line-of-sight with the subject's eyes, a camera is situated and setup to record movements in the subject's limbs and face).

According to some exemplary embodiments, the sensors data is recorded while the patient is at rest. Alternatively, or additionally, the sensors data is recorded while the patient participates in a task. In some embodiments, at first the data is recorded while the patient is at rest and then during task participation, or vice versa. In some embodiments, the recording during rest or during task participation is repeated more than once and the number of times the recording is performed is predefined, or may be modified online by results of previous recordings.

According to some exemplary embodiments, participation in a task comprises performing at least one motor task for example, using one of limb, repeatedly tapping the index finger and the thumb to each other. In some embodiments, participation in a task comprises articulating a set of syllables or words, and/or moving the eyes to each side. In some embodiments, participation in a task comprises moving one of the limbs of the patient by a device while the patient remains passive.

According to some exemplary embodiments, the patient performs a complex task, which optionally includes eye movement, limb movement and/or articulation. In some embodiments, the complex task is performed in interaction with a computerized display, e,g, a touch-sensitive tablet, on which instructions are explicitly or implicitly displayed. For example, in some embodiments, the subject is required to follow with their gaze a moving marker “A” on the display, to tap on it when it changes its appearance to marker “B” and to articulate when it changes to marker “C”.

According to some exemplary embodiments, the sensor signals are processed to obtain signal features. In some embodiments, for each attribute, an index is calculated by combining the signal features according to an equation.

Reference is now made to FIG. 4A, depicting a process for quantitative assessment of patient symptoms following task performance, according to some exemplary embodiments of the invention.

According to some exemplary embodiments, one or more sensor is applied to patient or patient environment at block 402. In some embodiments, the sensors comprise one or more of body sensors, optic sensors and environment sensors, for example as described in FIG. 2A.

According to some exemplary embodiments, signals from the sensors are recorded while a patient is at rest, for example when the patient is not involved in any physical and/or cognitive activity, for example that generates a movement of the patient or resist a movement applied on the patient by an external source, at block 404.

In some embodiments, the patient is instructed to be still and relaxed, not to move, not to help and not to resist an attempt from someone or something else to move the patient body.

According to some exemplary embodiments, signals from the sensors are recorded while the patient participates in a task, at block 406. In some embodiments, the task comprises performing at least one motor task for example, using one of limb, repeatedly tapping the index finger and the thumb to each other, opening and closing fist, holding an arm in the air, bringing a cup towards the mouth or moving the hand in a spiral shape. In some embodiments, participation in a task comprises articulating a set of syllables or words, and/or moving the eyes to each side. In some embodiments, participation in a task comprises moving one of the limbs of the patient by a device while the patient remains passive. In some embodiments, participation in a task comprises walking or running, standing stable without moving, or being pushed or pulled and regaining balance. In some embodiments a task is executed in combination with a cognitive challenge such as performing arithmetic calculations.

According to some exemplary embodiments, the patient performs a complex task, which optionally includes eye movement, limb movement and/or articulation. In some embodiments, the complex task is performed in interaction with a computerized display, e,g, a touch-sensitive tablet, on which instructions are explicitly or implicitly displayed. For example, in some embodiments, the subject is required to follow with their gaze a moving marker “A” on the display, to tap on it when it changes its appearance to marker “B” and to articulate when it changes to marker “C”.

According to some exemplary embodiments, the task is selected to provoke an appearance of at least one side effect of a treatment and/or at least one disease symptom. For example, Parkinson's Disease rigidity in one hand is known to often increase when the other hand is being used, for example when the first is opened and closed. Another example, often hand tremor in Essential Tremor appears while the patient is attempting to perform an accurate task with that hand, such as drinking or touching the clinician's finger with their own finger. Conversely, in Parkinson's Disease tremor tends to appear while the patient is at rest and often is reduced or eliminated when a movement is initiated.

According to some exemplary embodiments, rest and task-related signals are processed to calculate at least one feature, for example a sign, a symptom, and/or side effect, at block 408. In some embodiments, rest related signals are processed to quantify Parkinson's Disease tremor, rigidity, internal capsule recruitment, and/or posture. In some embodiments, task-related signals are processed to quantify bradykinesia, gaze palsy or diplopia, dysarthria or abnormal speech volume control and/or gait disorders.

According to some exemplary embodiments, an index for each feature is calculated at block 410. In some embodiments, an index, for example a score, is calculated for each feature. In some embodiments, the index is calculated using one or more algorithm stored in a memory of an assessment device, for example memory 216 shown in FIG. 2A or memory 314 shown in FIG. 3. In some embodiments, the index is calculated, for example by the analysis circuitry 218 shown in FIG. 2A, or by the index calculation module 320 shown in FIG. 3.

According to some exemplary embodiments, an overall score for the patient condition is calculated at block 412. In some embodiments, the overall score is calculated based on calculated index for each feature. In some embodiments, the overall score is calculated by a processor, a control circuitry or an analysis circuitry of the assessment device. In some embodiments, the overall score is calculated using at least one algorithm stored in a memory of the device.

Reference is now made to FIG. 4B depicting quantitative assessment of patient condition based on information from a large data set, according to some exemplary embodiments of the invention. In some embodiments, the information based on the large data set is generated using statistical inference and/or machine learning or any other classification, indexing, processing, scoring method described in this application.

According to some exemplary embodiments, sensor data from a plurality of patients is recorded, during rest and while performing a task, at block 414.

According to some exemplary embodiments, signal features are calculated at block 416 for the sensor data recorded at block 414. In some embodiments, signal features are data extracted from at least one stored signal. In some embodiments, the signal features are the at least one signal, for example in raw form. Alternatively or additionally, the features comprise a pre-processed form, for example after at least one of filtering, mean subtraction, artifact rejection or removal, noise cleaning, or similar processing methods that improve the usability of the signal while not significantly compressing its size or changing its nature.

According to some exemplary embodiments, the features are parameters extracted from the signal, for example mean, median, variance, standard deviation, statistical skewness, kurtosis or other high-order statistical measures, Discrete Cosine Transform (DCT) components and/or entropy. In some embodiments, spectral domain features include one or more of frequency of highest spectral power component, magnitude of highest spectral power component, total harmonic distortion, the power in one or more frequency bands that can be calculated as an integral of the PSD of the signal between the two edges of the frequency band, statistical properties or measures of the PSD.

According to some exemplary embodiments, features are constructed from time-frequency representations of the signal, for example short time Fourier transforms, other Fourier-based spectrograms, for example based on Welch spectrum estimations, wavelet transforms, Wigner-Ville transforms or similar transforms. In some embodiments, features are constructed from entire time-frequency representations, from at least one selected segment in the time-frequency representations, or from selected components of these representations, for example the magnitude at one or more bands during one or more time intervals, or duration or power of a continuous peak or trough in the time-frequency domain. Alternatively or additionally, other features are driven from cepstral analysis (equivalent in some embodiments to applying spectral estimation to the log of the PSD), including cepstral coefficients, and/or mel-Frequency Cepstral Coefficients (MFCCs).

According to some exemplary embodiments, features comprise parametric representations of the signals, for example auto-regressive (AR) coefficients that optionally provides an optimal estimate of the signal, auto-regressive moving average (ARMA) coefficients that optionally optimally estimates the signal, Linear Prediction Coding coefficients, or other parametric representations. In some embodiments, the features are of a higher-order, that is to be constructed from more than one signal, for example from 2 or more EMG channels, or between at least 1 EMG channel, at least 1 kinematic sensor (accelerometer, gyroscope, goniometer or an optic marker picked up by a camera serving to track a movement of the patient) or between any 2 or more data channels. In some embodiments, high-order features comprise mutual information between signals, correlation coefficients between pairs of signals, maximal cross-correlation value between 2 signals, latency between signals (for example estimated by the lag corresponding to the maximal cross-correlation).

According to some exemplary embodiments, features are derived from other features, instead of directly from the signals. In some embodiments, features are constructed for example, from dimension reduction methods that combine multiple inputs (primary features) optionally optimizing a goal function that generally attempts to concentrate the “important information” in a smaller number of components than the number of inputs. In some embodiments, assuming there are N inputs, that could be N features from the list described above, there usually also N outputs, but what the goal function defines as “important information” is concentrated in M<N output components. In some embodiments, for example, the principal component analysis (PCA) method's goal function defines the data variance as the important information, and for some cases, 3 principal components, calculated by a linear combination of for example 100 inputs, can be enough to account for 90% or 95% of the variance in the data. Thus, it is possible to maintain only 3 PCA components and these would be features for subsequent analysis.

According to some exemplary embodiments, additional techniques for dimensionality reduction comprise the Non-negative Matrix Factorization (NMF), Local Linear Embedding (LLE), Laplacian template maps, Isomaps, Linear Discriminant Analysis, Generalized Discriminant Analysis, Maximum Variance Unfolding and diffusion maps.

According to some exemplary embodiments, a human specialist assessment for symptoms and side effects in a plurality of patients is provided, at block 418. In some embodiments, a database of labeled data is constructed based on the human specialists assessments. In some embodiments, the human specialist assessments serve as a reference, for example a “ground truth” labels, that the algorithms attempt to match, for example through optimizing the combination of signal features. In some embodiments, as this process continues and the number of patients from which data is collected grows, the accuracy improves.

According to some exemplary embodiments, relation of signal features to each symptom and side effect is statistically inferred, at block 420. In some embodiments, a relation between the features and the outputs is estimated. In some embodiments, the outputs are the human expert assessments, for example in a form of binary variables (side-effect is present or not), or categorical variables, as symptoms. In some embodiments, for example, Parkinson's Disease symptoms, are assessed based on a rating scale, for example the unified Parkinson's disease rating scale (UPDRS) or its variants, in which each assessment is actually a categorization of the symptom or side effect to one of groups, defined as 0, 1, 2, 3 and 4.

According to some exemplary embodiments, methods to estimate the relation between features and outputs comprise linear regression or regression analysis in general, logistic regression for binary variables, perceptrons and multi-player perceptrons, support vector machines, Naïve Bayes classifier, k-nearest neighbors, decision trees and random forests, artificial neural networks (ANNs) including deep neural networks, recurrent neural networks and convolutional neural networks, Bayesian networks including dynamic Bayesian Networks (DBNs) and Hidden Markov Models (HMMs), genetic algorithms and evolutionary algorithms. In some embodiments, the algorithms and models in general attempt to optimize the correctness of the prediction of the correct output based on the inputs. Optionally, the prediction is optimized by training algorithms, that train by repetitive updating the model parameters (such as the connections between nodes in an ANN, the probability matrix in a HMM, and so on) according to an update rule, until converging to an optimum in which the prediction error is smallest. In some embodiments, for example, ANNs are trained by Backward Propagation techniques, HMMs are trained by the Viterbi algorithm, and Bayesian Networks by Belief Propagation methods.

In some embodiments, in some of these models, the degree that each input feature is important in improving the prediction and minimizing error, is established explicitly, for example in linear regression. In other techniques, such as ANNs, it is not explicitly clear how each feature contributes to minimizing prediction error. In some embodiments, by removing an input feature, repeating the training process and calculating the prediction error without the removed feature, it is possible to rank the features according to their impact on the prediction error. In some embodiments, this process is also performed for pairs, triplets, and so forth of features, as in some cases the combination of features together is more informative than the sum of their informative values.

In some embodiments, the extent to which a feature or combination of features contributes to the prediction, is dependent on other variables, for example the patient's disease, disease stage, age, dominant symptoms, dominantly affected side and additional drugs or other medications. In some embodiments, by having a large enough database, it is possible to evaluate the most informative features for a subset of the patient population, for example a specific combination of two or more of disease, disease stage, age, dominant symptoms, dominantly affected side and additional drugs or other medications.

According to some exemplary embodiments, the most informative signal features types are selected at block 422. In some embodiments, most informative refers to a set of feature types that is found, for example by testing on previously obtained data, to be most useful in calculating a specific index accurately. In some embodiments, this is based on obtaining previous data from the same patient, or previous data from other patients, or a database, in which the data is also accompanied by labels that were generated externally, not by the system, and indicate the patient condition. Often these labels can be provided by expert clinicians that have examined the patient at the same time or at an equivalent condition as the system. Based on such labels, it is possible to test manually, or to use automatic algorithms, in order to determine the most informative signal features types for a specific combination of disease, disease stage, age, dominant symptoms, dominantly affected side and additional drugs or other medications. In some embodiments, the calculation methods for calculating features in block 416, and the list of most informative signal features types determined in block 422 are applied at block 408 on the recordings from blocks 404 and 406.

Alternatively or additionally, most informative signal features are used to update one or more index calculation formula or algorithm, at block 424. In some embodiments, the index calculation formula or algorithm is the specific formula, or model, or algorithm, as described above as relating between the signal features and the experts' assessments. In some embodiments, after selecting the most informative features, the formula, model, or algorithm trained based on the selected M input features, is the updated index calculation formula, or an index calculation method.

According to some exemplary embodiments, the updated one or more index calculation formula or algorithm from block 424 is used for index calculation at block 410. Thus, an index calculation method constructed and trained over a database consisting of data acquired from the same patient in the past, and/or other patients, is used to calculate the index for the specific patient in the present during the assessment procedure.

### Exemplary Quantitative Assessment of PD Symptoms and Treatment Side Effects

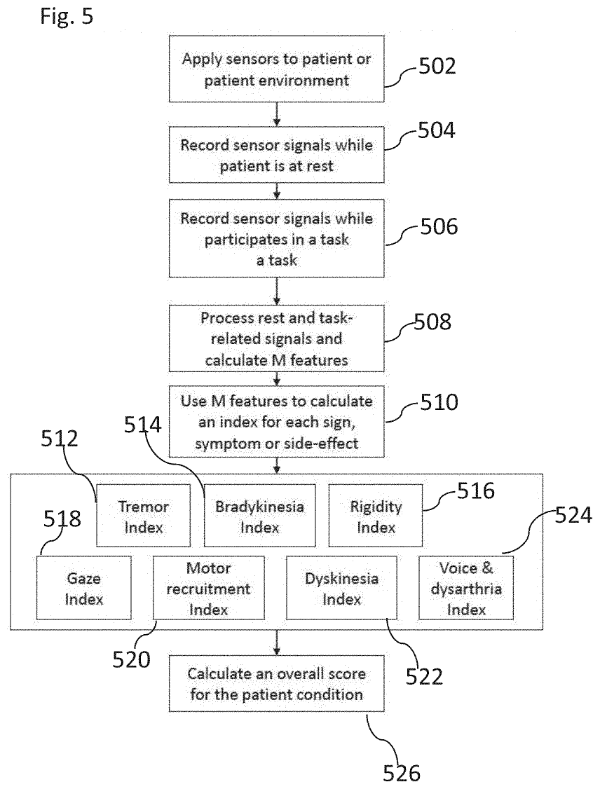

Reference is now made to FIG. 5 depicting a process for quantitative assessment of neurological disease symptoms, for example PD, and/or treatment side effects, according to some exemplary embodiments of the invention.

According to some exemplary embodiments, at least one sensor is applied to a patient or patient environment, at block 502. In some embodiments, the at least sensor comprises one or more of a body sensor, an optic sensor, and/or an environment sensor.

According to some exemplary embodiments, signals are recorded by the at least one sensor while the patient is at rest, at block 504.

According to some exemplary embodiments, signals are recorded by the at least one sensor while the patient participates in a task, at block 506.

According to some exemplary embodiments, rest and task-related signals are processed at block 508. In some embodiments, the signals are processed, for example to calculate one or more features.

According to some exemplary embodiments, the one or more calculated features are used to calculate an index for each sign, symptom and/or side effect, at block 510. In some embodiments, a specific index is calculated, for example a tremor index 512, a bradykinesia index 514, a rigidity index, a gaze index 518, a motor recruitment index, a dyskinesia index and/or a voice and dysarthria index 524.

According to some exemplary embodiments, an overall score for the patient condition is calculated at block 526. In some embodiments, the overall score is calculated based on at least some of the specific indices.

### Exemplary Pulse Generator Programming

According to some exemplary embodiments, a pulse generator, for example an implanted pulse generator (IPG) is programmed based on a quantitative assessment of a patient condition. In some embodiments, the IPG is programmed automatically by a device or a system for assessment of a patient condition. Alternatively, the IPG is programmed manually by an expert, for example a physician or a nurse based on recommendations, for example recommended treatment parameter values delivered to the expert by the assessment device.

According to some exemplary embodiments, a method for programing an IPG for delivering DBS comprises the following steps.

According to some exemplary embodiments, data from the DBS implantation surgery and previous programing sessions if they exist—prior data is received. In some embodiments, the prior data includes electrophysiology recordings from the surgery and/or processed outputs of these recordings mapping the recorded trajectories to functional territories as described for example in U.S. Pat. No. 8,792,972 or WO2018008034. According to some exemplary embodiments, the prior-data is used to plan an efficient search of the DBS parameter space. In some embodiments, the search includes identifying DBS lead contacts that are positioned in statistically less-beneficial positions and that should not be tested or should be tested relatively sparsely, as well as optimally positioned contacts that should be tested at high resolution. In some embodiments, the plan includes which DBS configurations will be tested.

According to some exemplary embodiments, the plan is presented to a caregiver of the patient and approval is obtained.

According to some exemplary embodiments, at least one sensor is applied to the patient and/or to the environment of the patient, for example to record one or more of voice, eye movement, muscle activation and mechanical proxies of rigidity.

According to some exemplary embodiments, all variables are recorded at baseline condition, with the patient OFF treatment or receiving baseline treatment.

According to some exemplary embodiments, an initial scan procedure begins.

According to some exemplary embodiments, the DBS parameters are adjusted to a selected planned configuration. In some embodiments, a DBS treatment is delivered to the patient using the selected configuration.

According to some exemplary embodiments, data from all sensors is recorded. In some embodiments, various analysis methods are applied on the recorded data, for example to obtain indices for one or more of the various symptoms, signs and side effects. Optionally, the analysis results and/or the indices are used to adjust the scan plan. In some embodiments, if a contact or contacts configuration expected to be highly beneficial leads to side-effects at relatively low current stimulation, higher voltages, or more fine-grained testing of this contact may be cancelled. Additionally or alternatively, a contact or contact configuration initially considered poor yielding desirable results can be scanned at finer details to search for an optimum. In some embodiments, once data recording with a first DBS parameters configuration is completed, the configuration is changed to a different configuration, DBS treatment is delivered and data from the sensors is measured. In some embodiments, the DBS configuration is changed until reaching the last planned configuration.

According to some exemplary embodiments, the scan results are presented, for example in the form of a table summarizing the quantification of attributes at each tested configuration, and/or by a higher-level graphical representation optionally highlighting onset of symptom alleviation and side effects per all or selected configurations. Optionally a group of most optimal configurations is presented, for example a group including at least 2, 3, 4, 5, 6 or any smaller or larger of configurations.

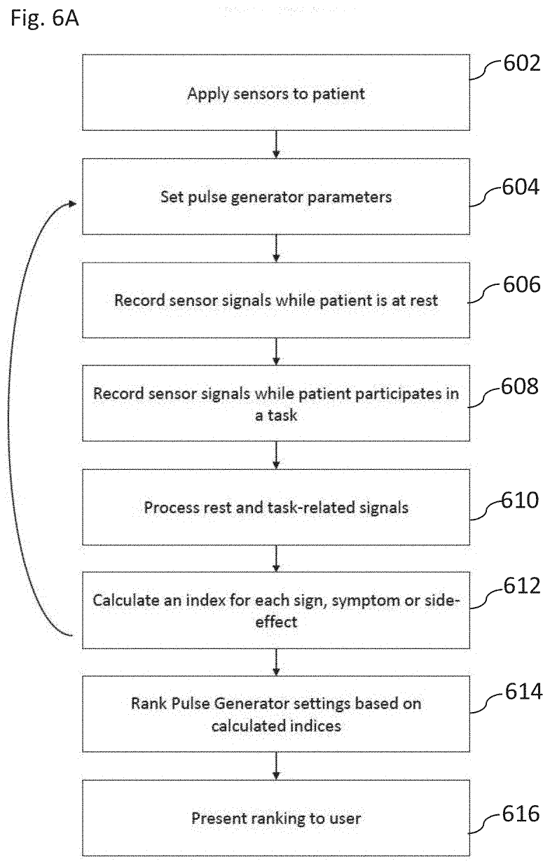

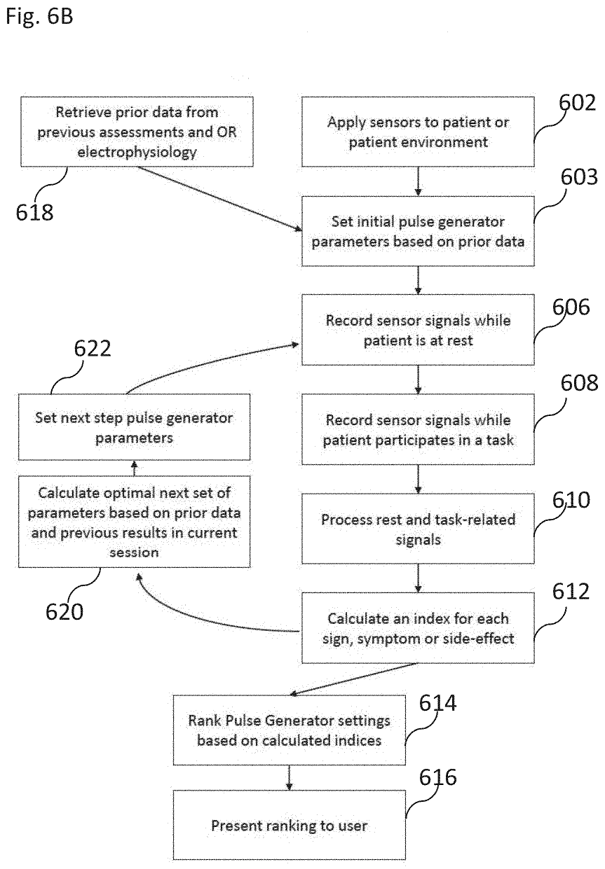

Reference is now made to FIG. 6A depicting pulse generator programming using quantitative assessment of patient symptoms, according to some exemplary embodiments of the invention.

According to some exemplary embodiments, sensors are applied to the patient and/or to the patient environment, at block 602. In some embodiments, the sensors comprise one or more of body sensors, optic sensors and environmental sensors.

According to some exemplary embodiments, pulse generator parameters are set at block 604. In some embodiments, once the parameters are set a DBS treatment is delivered to the patient.

According to some exemplary embodiments, sensor signals are recorded while the patient is at a rest state, at block 608.

According to some exemplary embodiments, sensor signals are recorded while the patient participates in a task, at block 610.

According to some exemplary embodiments, signals recorded during rest and during participation in a task are processed, at block 610.

According to some exemplary embodiments, an index is calculated for each sign, symptom or side effect, at block 612. In some embodiments, the index is calculated based on the processed signals.

According to some exemplary embodiments, once the index is calculated, the pulse generator parameters are set with a different set of values, at block 604. In some embodiments, signal recordings, signal processing and calculation of a new index for each sign, symptom or side effect, is repeated while the patient receives a DBS treatment with the new set of parameter values.

According to some exemplary embodiments, the different settings of the pulse generator are ranked based on the calculated indices, at block 614.

According to some exemplary embodiments, the ranking is presented to the user. Alternatively, the pulse generator is automatically programmed according to a selected setting, for example a setting that has the highest ranking.

According to some exemplary embodiments, for example as shown in FIG. 6B, prior data from previous assessments and operating room electrophysiology is retrieved at block 618. In some embodiments, the prior data is stored in a memory of the assessment device.

According to some exemplary embodiments, initial pulse generator parameters are set based on the stored prior data, at block 603. In some embodiments, once the parameters are set, a DBS is delivered to the patient using the initial pulse generator parameters.

According to some exemplary embodiments, once an index is calculated at block 612, the next set of parameters is calculated based on prior-data and/or previous results in current session, at block 620. In some embodiments, the next set of parameters is the optimal set of parameters, in the sense that it is the set of parameters most likely to be the most efficient set to select at this stage, and to minimize the number of subsequent parameter sets that would be tested before reaching an optimal set of parameters that lead to a DBS treatment with a maximal therapeutic effect and minimal side effects.

According to some exemplary embodiments, the pulse generator is set with the next set of parameters at block 622. In some embodiments, once the pulse generator is programmed with the next set of parameters, DBS is delivered to the patient.

### Exemplary Index Calculation

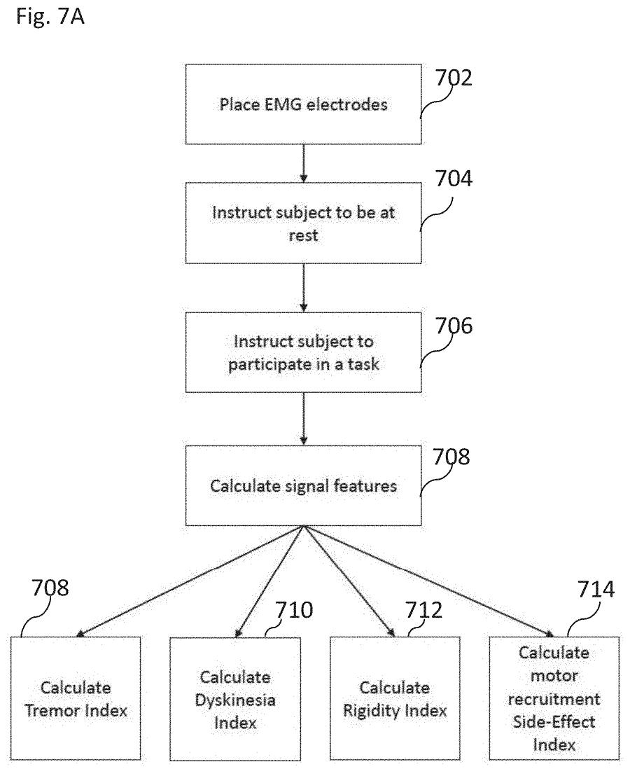

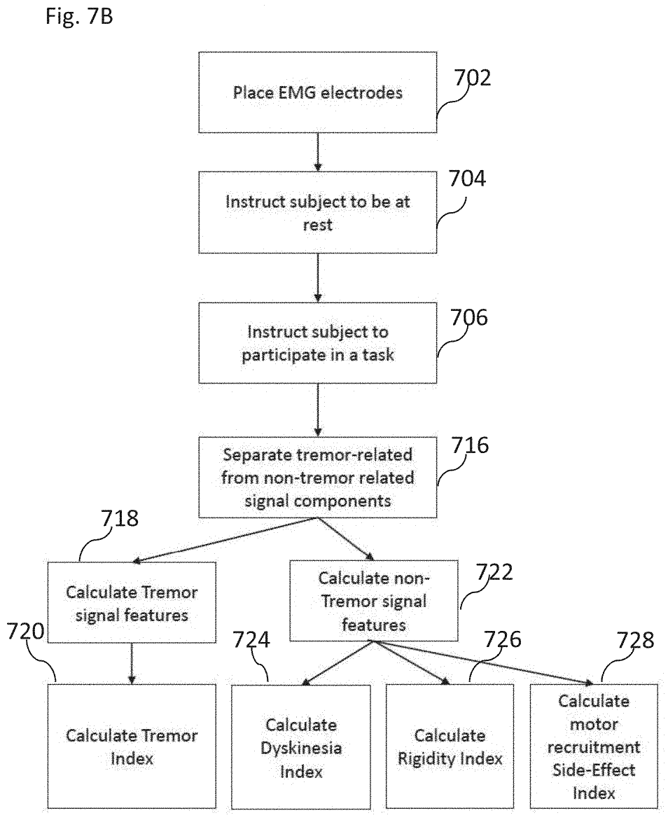

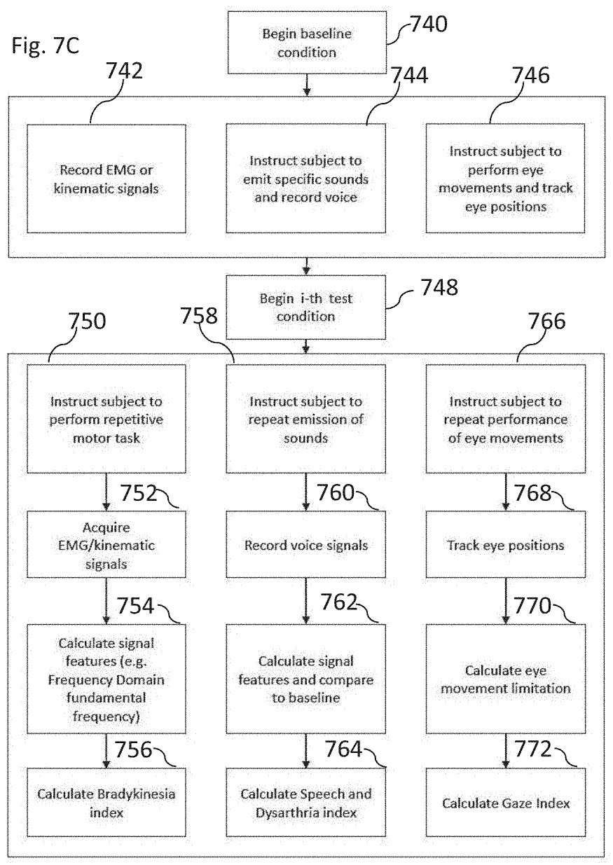

Reference is now made to FIG. 7A, depicting a general process for generation of one or more index, according to some exemplary embodiments of the invention.

According to some exemplary embodiments, EMG electrodes are placed on a subject, for example a patient, at block 702. In some embodiments, the EMG electrodes are placed at one or more location on face of the patient. Alternatively or additionally, the electrodes are placed at one or more location on at least one limb of the subject, for example a leg or a hand.

According to some exemplary embodiments, a subject is instructed to be at rest, at block 704. In some embodiments, when the subject is at rest, signals from the EMG electrodes are received and optionally processed and/or stored.

According to some exemplary embodiments, a subject is instructed to participate in a task, at block 706. In some embodiments, when the subject participates in a task, signals from the EMG electrodes are received, and optionally processed and/or stored.

According to some exemplary embodiments, signal features are calculated at block 708. In some embodiments, the signal features are calculated from the signals measured when the subject was at rest and/or from the signals measured when the subject participated in a task.

According to some exemplary embodiments, one or more indices are calculated from the calculated signal features. In some embodiments, a tremor index is calculated at block 708. In some embodiments, a dyskinesia index is calculated at block 710. In some embodiments, a rigidity index is calculated at block 712. In some embodiments, a motor recruitment side-effect index is calculated at block 714.

According to some exemplary embodiments, tremor-related signal components are separated from non-tremor related signal components prior to calculation of signal features. Reference is now made to FIG. 7B, depicting a process for index generation with separation of tremor-related signals, according to some exemplary embodiments.

According to some exemplary embodiments, following separately recording signals in rest and while a subject participates in a task, tremor-related signals, for example signal components, are separated from non-tremor related signals, at block 716.

According to some exemplary embodiments, tremor signal features are calculated at block 718, from tremor-related signal components separated at block 716. In some embodiments, a tremor index is calculated at block 720. In some embodiments, the tremor index is calculated from the calculated tremor signal features.

According to some exemplary embodiments, the non-tremor signal components separated at block 716, are used to calculate non-tremor signal features at block 722. In some embodiments, a dyskinesia index is calculated at block 724 based on the calculated non-tremor signal features. In some embodiments, a rigidity index is calculated at block 726 based on the calculated non-tremor signal features. In some embodiments, a motor recruitment side effect index is calculated at block 728 based on the calculated non-tremor signal features.

According to some exemplary embodiments, the non-tremor related indices, for example the dyskinesia index, the rigidity index and the motor recruitment side-effect index are calculated separately from the calculated non-tremor related signal.

### Exemplary Task-Related Index Calculations

Reference is now made to FIG. 7C depicting task-related index calculations compared to a baseline, according to some exemplary embodiments of the invention.

According to some exemplary embodiments, baseline measurements of one or more of disease symptom, treatment side effect and subject condition are initiated at block 740. In some embodiments, during the baseline measurements, EMG or kinematic signals are recorded from a subject at block 742. In some embodiments, the EMG or kinematic signals are recorded while the subject is at rest. Alternatively or additionally, the subject is instructed to emit specific sounds and the emitted sounds are then recorded. Alternatively or additionally, the subject is instructed to perform eye movements while the system records the eye position and/or tracks the eye movement.

According to some exemplary embodiments, following or during treatment delivery or after a selected period of time from the baseline measurements, test condition measurements are performed at block 748.

According to some exemplary embodiments, the subject is instructed to perform a repetitive motor task at block 750, for example as described above. In some embodiments, the subject is instructed to perform the task during the delivery of a treatment, or within a time period of up to 1 day, for example up to 12 hours, up to 10 hours, up to 5 hours, up to 1 hour, up to 30 minutes or any intermediate, smaller or larger time period from the ending of the treatment delivery.

According to some exemplary embodiments, EMG and/or kinematic signals are acquired at block 752. In some embodiments, the signals are acquired during the performance of the motor task. Alternatively, the signals are acquired in a time duration of up to 1 day, for example up to 12 hours, up to 10 hours, up to 5 hours, up to 1 hour, up to 30 minutes or any intermediate, smaller or larger time duration from the ending of the motor task performance.

According to some exemplary embodiments, one or more signal features are calculated at block 754. In some embodiments, the one or more signal features comprise frequency, domain fundamental frequency, or other signal features described in the section “exemplary feature construction”. In some embodiments, the features are calculated from the EMG or kinematic signals recorded at rest, and following or during the performance of the motor task. In some embodiments, the calculated features of signals measured during or following a task are compared to calculated features of base line signals, for example signals measured at rest.

According to some exemplary embodiments, a Bradykinesia index is calculated at block 756. In some embodiments, the Bradykinesia index is calculated based on the comparison to baseline features as described at block 754.

According to some exemplary embodiments, the subject is instructed to repeat the emission of sounds, at block 758. In some embodiments, the subject is instructed to repeat the emission of sounds during the delivery of a treatment or within a time period of up to 1 day, for example up to 12 hours, up to 10 hours, up to 5 hours, up to 1 hour, up to 30 minutes or any intermediate, smaller or larger time period from the ending of the treatment delivery.

According to some exemplary embodiments, voice signals are recorded at block 760. In some embodiments, the signals are acquired during the emission of the sounds. Alternatively, the signals are acquired in a time duration of up to 1 day, for example up to 12 hours, up to 10 hours, up to 5 hours, up to 1 hour, up to 30 minutes or any intermediate, smaller or larger time duration from the ending of the sounds emission.

According to some exemplary embodiments, signal features are calculated at block 762. In some embodiments, the signal features are calculated from the baseline signals and from the voice signals recorded at 760. In some embodiments, the calculated features of the base signals are compared to the calculated features of the signals recorded at block 760.

According to some exemplary embodiments, a speech and/or dysarthria index is calculated at block 764. In some embodiments, the speech and/or dysarthria index is calculated based on the results of the comparison performed at block 762.

According to some exemplary embodiments, a subject is instructed to repeat performance of eye movements, at block 766. In some embodiments, the subject is instructed to repeat the performance of eye movements during the delivery of a treatment, or within a time period of up to 1 day, for example up to 12 hours, up to 10 hours, up to 5 hours, up to 1 hour, up to 30 minutes or any intermediate, smaller or larger time period from the ending of the treatment delivery.

According to some exemplary embodiments, eye positions are tracked at block 768. In some embodiments, the eye positions are tracked during the performance of the eye movements at block 766. Alternatively, eye positions are tracked in a time duration of up to 1 day, for example up to 12 hours, up to 10 hours, up to 5 hours, up to 1 hour, up to 30 minutes or any intermediate, smaller or larger time duration from the ending of the eye movement performance at block 766.

According to some exemplary embodiments, an eye movement limitation is calculated at block 770. In some embodiments, the eye movement limitation is calculated based on the comparison between baseline eye positions and the eye positions following the repeated performance of eye movements.

According to some exemplary embodiments, a gaze index is calculated at block 772. In some embodiments, the gaze index is calculated based on the calculated movement limitation. In some embodiments, the gaze index is calculated based on the difference between the eye positions recorded at baseline and the eye positions following repeated performance of eye movements.

### Exemplary Separation of Tremor-Related Signals

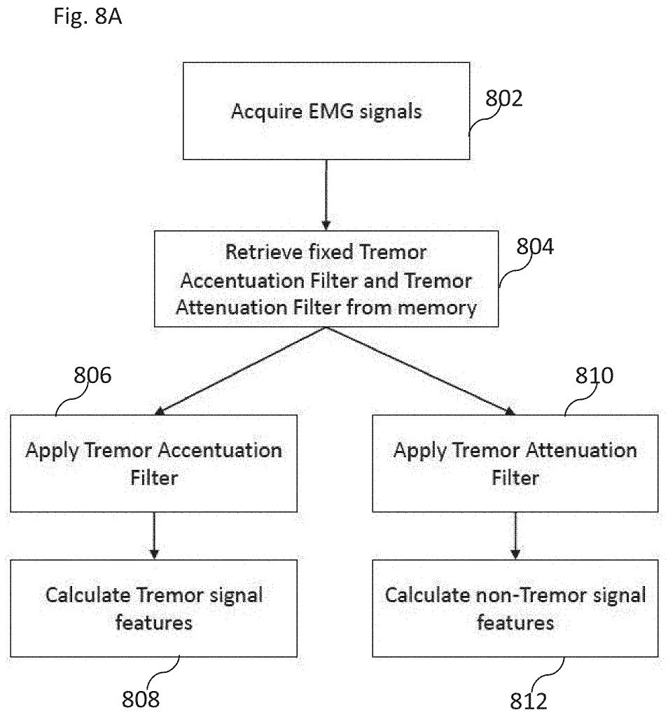

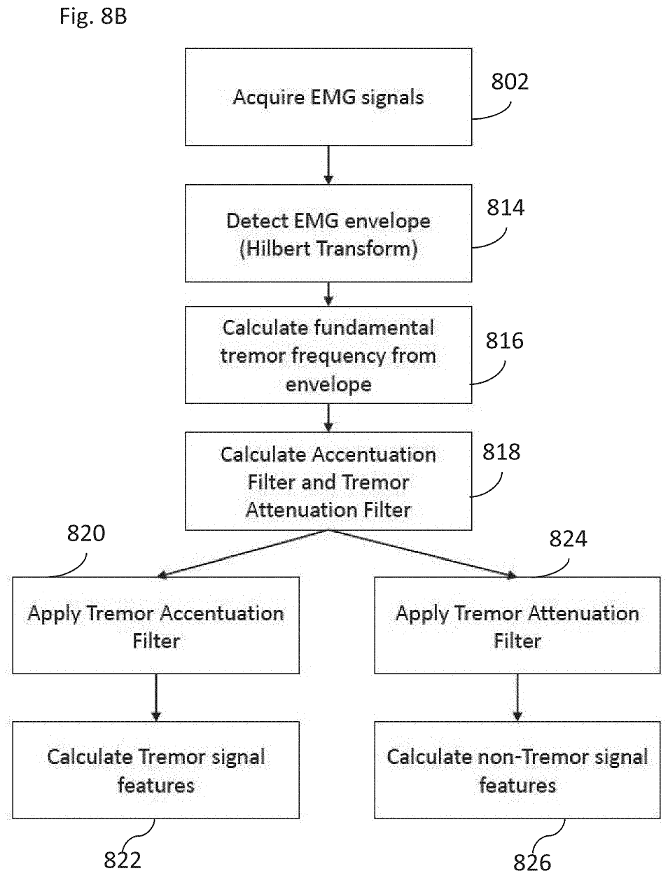

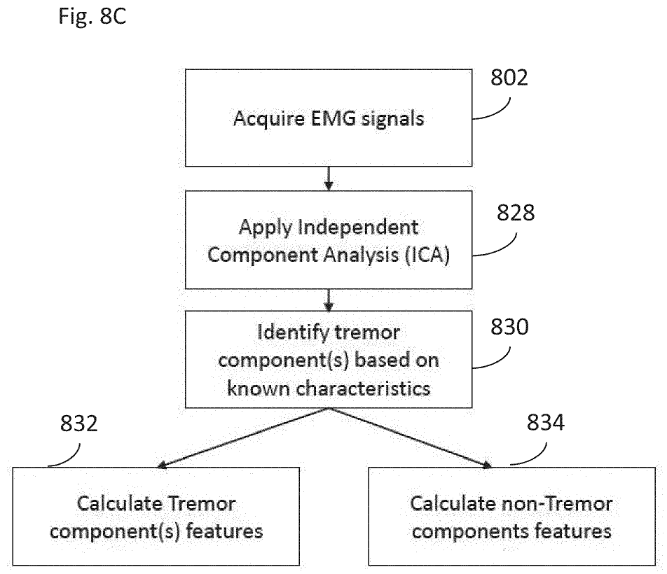

According to some exemplary embodiments, signals recorded by at least one sensor are separated into tremor-related signals and non-tremor related signals, prior to calculation of signal features and/or calculation of at least one index, for example as shown in FIG. 7B. Reference is now made to FIGS. 8A-8C depicting different methods for separation of tremor-related signals from non-tremor related signals according to some exemplary embodiments of the invention.

According to some exemplary embodiments, signals are acquired from at least one sensor, for example at least one EMG sensor, at block 802. In some embodiments, the acquired signals are stored in a memory of an assessment device, for example memory 216 or memory 314 shown in FIGS. 2A and 3 respectively.

According to some exemplary embodiments, fixed tremor accentuation and fixed tremor attenuation filters are retried from a memory, at block 804.

According to some exemplary embodiments, the tremor accentuation filter is applied on the acquired sensor signals, at block 806. In some embodiments, tremor signal features are calculated based on the filtered accentuated signals, at block 808.

According to some exemplary embodiments, the tremor attenuation filter is applied on the acquired sensor signals, at block 810. In some embodiments, non-tremor signal features are calculated based on the filtered attenuated signals, at block 812.

According to some exemplary embodiments, for example as shown in FIG. 8B, adaptive filtering is applied on the recorded signals to separate tremor-related signals from non-tremor related signals.

According to some exemplary embodiments, the sensor signals acquired at block 802 comprise EMG signals.

According to some exemplary embodiments, an EMG envelope is detected in the acquired EMG signals. In some embodiments, the EMG envelope is detected by applying a Hilbert transformation algorithm on the acquired EMG signals.

According to some exemplary embodiments, tremor frequency, for example fundamental tremor frequency is calculated form the detected envelope at block 816.

According to some exemplary embodiments, an accentuation filter and/or an attenuation filter are calculated at block 818. In some embodiments, the one or both filters are calculated based on the tremor frequency calculated at block 816.

According to some exemplary embodiments, the calculated tremor accentuation filter is applied on the acquired EMG signals at block 820. In some embodiments, tremor signal features are calculated at block 822. In some embodiments, the tremor signal features are calculated based on accentuated tremor signals filtered at block 820.

According to some exemplary embodiments, the calculated tremor attenuation filter is applied on the acquired EMG signals at block 824. In some embodiments, non-tremor signal features are calculated at block 826. In some embodiments, the non-tremor signal features are calculated based on attenuated tremor signals filtered at block 824.

According to some exemplary embodiments, for example as shown in FIG. 8C independent component analysis (ICA) is applied on acquired sensor signals, prior to features calculation for example EMG signals.

According to some exemplary embodiments, an independent component analysis is applied on acquired EMG signals at block 828.

According to some exemplary embodiments, tremor components are identified in the results of the of the ICA analysis, based on known characteristics of the output ICA components, for example amplitude, fundamental frequency, harmonic distortion, entropy, kurtosis, at block 830.

According to some exemplary embodiments, an output ICA component that has characteristics most similar to typical characteristics for the symptom-related component is identified. In some embodiments, the identified tremor components are stored separately from the non-tremor components.

According to some exemplary embodiments, tremor components features are calculated at block 832. In some embodiments, the tremor components features are calculated based on the identified tremor components.

According to some exemplary embodiments, non-tremor components features are calculated at block 834. In some embodiments, the tremor components features are calculated based on the identified non-tremor components.

### Exemplary Tremor Analysis

According to some exemplary embodiments, processing of tremor-related signals is performed in different processing methods. In some embodiments, EMG signals are received and processed in order to detect tremor. In some embodiments, the signal processing comprises an “on demand” signal processing, for example a signal processing initiated in response to an indication, for example a signal from a control circuitry or a user. In some embodiments, an “on demand” signal processing is used to quantify tremor from an EMG signal recorded at a specific site, for example at one or more of left face, right face, left upper limb, left lower limb, right upper limb, right lower limb.

According to some exemplary embodiments, the processing method is also used for acquisition and signal processing for other symptoms and/or side effects. In some embodiments, the patient is required to perform different tasks or be at rest, when recording signals for detection of at least one side effect and/or at least one disease symptom. In some embodiments, at each stimulation level (or other therapy level), the patient is examined for various symptoms and/or side-effects, such that the signals that should be processed to obtain an index for a specific symptom—tremor in this example—do not arrive continuously, but rather the processing should be performed “on demand”. In some embodiments, an indication from a user or a control circuitry initiates recording signals from at least one sensor to evaluate at least one side effect and/or at least disease symptom. Alternatively, the indication from a user or a control circuitry marks a window in previously recorded signals from at least one sensor, for example to evaluate the at least one side effect and/or the at least disease symptom using signals within the marked window.

According to some exemplary embodiments, the system waits for an indication, for example a flag, or trigger, to signal that the EMG signals being acquired are to be used as input for a specific signal processing process, for example tremor signal processing. In some embodiments, the indication is generated automatically by the system. In some embodiments, when the system detects that the patient is at rest, the system generates an indication that the current recorded EMG signals should be used for one or more of tremor, inter capsule recruitment and EMG-based rigidity. In some embodiments, when the system detects that the patient moves his eyes in a stereotypical manner, for example, moving the eyes in large movement to one side, and then a large movement to the other side, the system indicates that the recorded signals should be used for Gaze-disorder processing.

According to some exemplary embodiments, processing Inertial Measurement Unit (IMU) signals for rigidity analysis is automatically triggered, for example by recognizing stereotypical, repetitive, large movements around the axis of the elbow. In some embodiments, processing the microphone signal for dysarthria identification is triggered, for example by recognizing a stereotypical articulation pattern that the patient emits for dysarthria testing. Alternatively, a user initiates the processing, either manually, by pressing a button or another input device to signal the required trigger, or orally, by saying the name of the tested symptom, which is picked up by the microphone and automatically identified by a speech processing circuitry in the system.

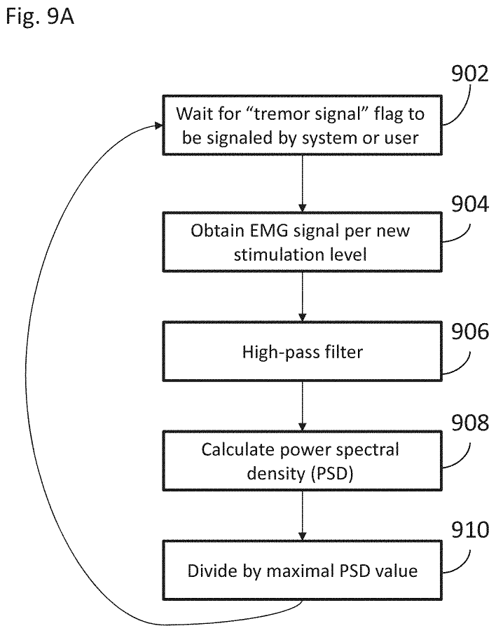

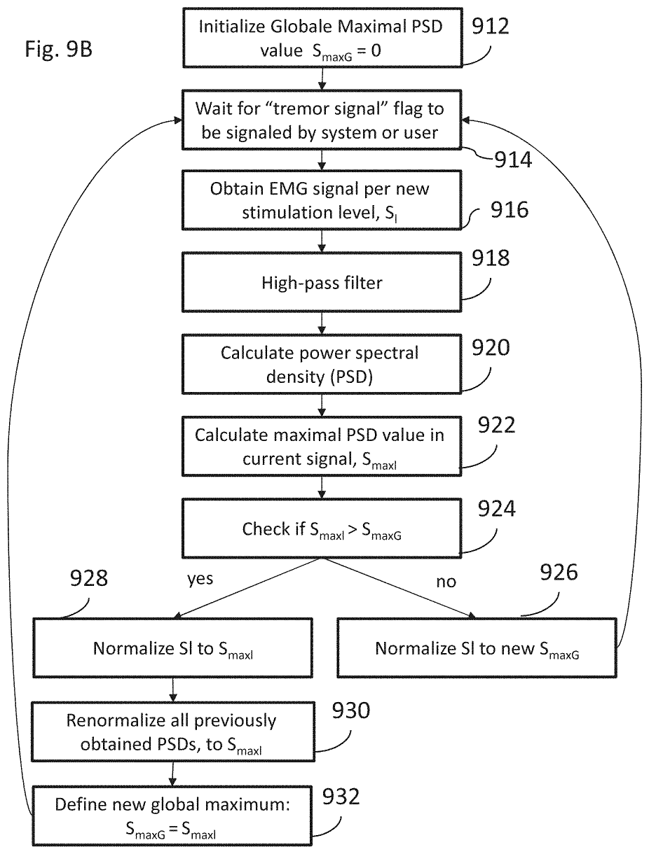

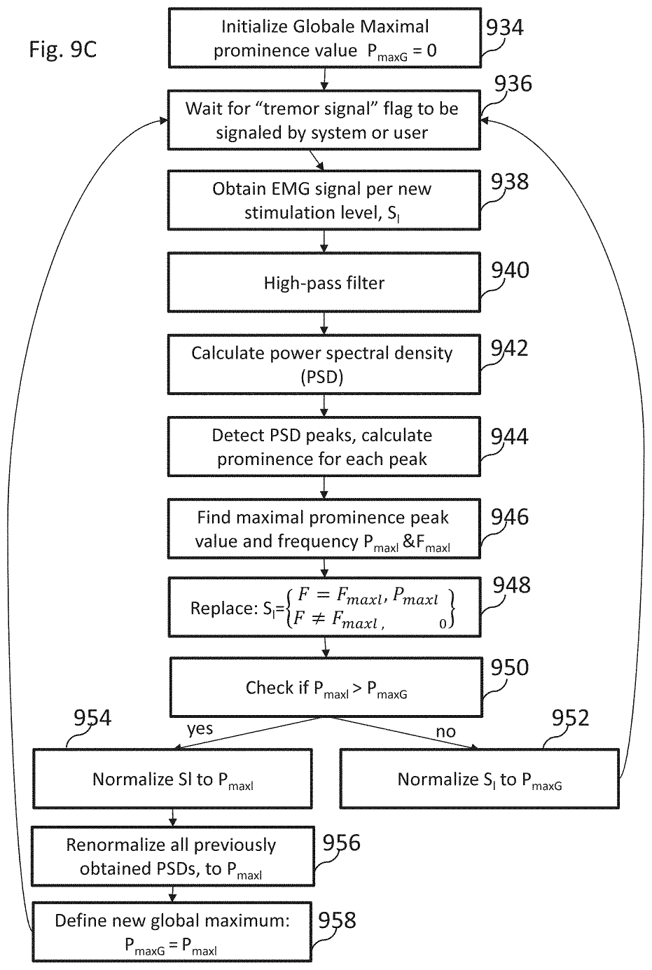

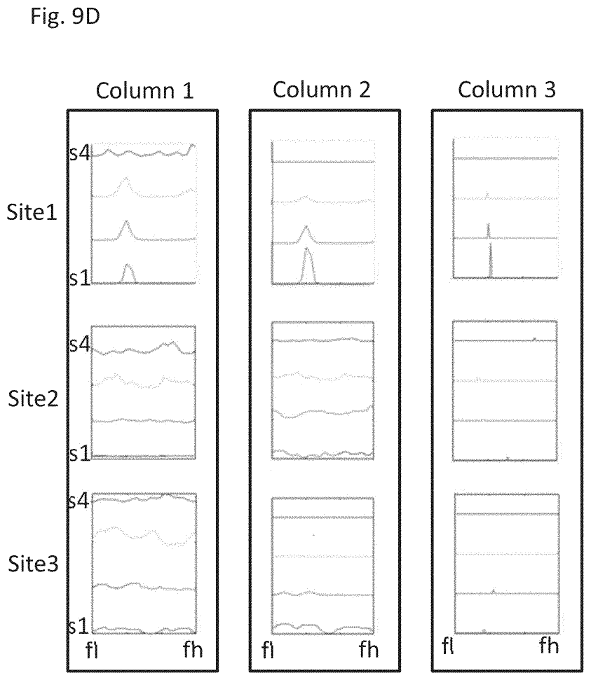

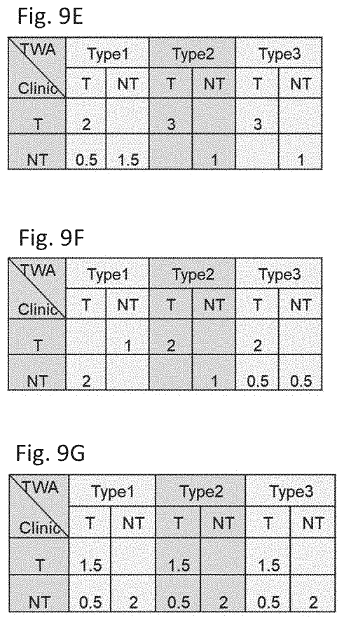

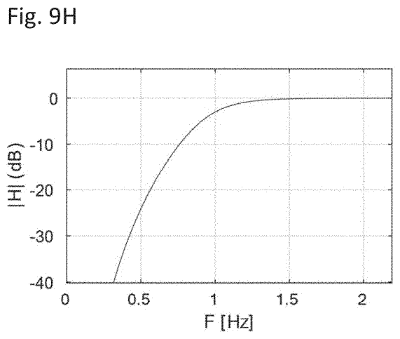

Reference is now made to FIG. 9A, depicting a general on-demand process for initiating analysis for detection of tremor, according to some exemplary embodiments of the invention.

According to some exemplary embodiments, an assessment system remains in a stand-by mode until an indication regarding tremor signals measurements is received, at block 902.

According to some exemplary embodiments, when an indication is received, at least one EMG signal is obtained, for example per a new stimulation level, at block 904.

According to some exemplary embodiments, the obtained signal is filtered using a high-pass filter, at block 906.

According to some exemplary embodiments, power spectral density (PSD) is calculated at block 908.

According to some exemplary embodiments, the PSD results are normalized, for example divided by a maximal value in PSD, at block 910.

According to some exemplary embodiments, the results of the PSD or following normalization is displayed in the user interface. In some embodiments, the information is presented in frequency ranges according to the detected side effect or disease symptoms, for example the displayed frequency range is in a range of 2-8 Hz (for PD tremor). In some embodiments, this allows a user to focus on the 3-7 Hz that is the frequency band of PD tremor. In some embodiments, for Essential Tremor (ET) patients, the displayed frequency range is in a range of 2-14 Hz, for example to allow view of the 4-12 Hz range of ET tremor. In some embodiments, these ranges are recommended for patients with typical tremors, however the UI is configured to modify the displayed range to better suit a specific patient or the preference of the user/clinician.

Reference is now made to FIG. 9B, depicting the use of a global maximal PSD value, according to some exemplary embodiments of the invention.

According to some exemplary embodiments, tremor is quantified in response to an indication, based on EMG recordings from one or more specific EMG sites, for example left face, right face, left upper limb, left lower limb, right upper limb, and/or the right lower limb.

According to some exemplary embodiments, a global maximal PSD value, SmaxG, is stored in memory (initiated as equals 0), for example for a specific EMG site, at block 912. In some embodiments, each EMG site has its own global maximum, and the analysis is performed for each site separately.

According to some exemplary embodiments, an assessment system is in a standby state, waiting for an indication from a system or user, at block 914.

According to some exemplary embodiments, upon receiving an indication, for example flag for processing another input EMG signal for tremor quantification, an EMG signal is obtained at block 916. In some embodiments, the EMG signal is obtained per a new stimulation level.

According to some exemplary embodiments, the obtained signal is filtered using a high-pass filter, at block 918.

According to some exemplary embodiments, PSD is calculated, Sl, at block 920.

According to some exemplary embodiments, a maximal PSD value in the current signal, Smaxl, is calculated at block 922.

According to some exemplary embodiments, the maximal value of the PSD of current stimulation level, Smaxl, is compared with SmaxG at block 924.

According to some exemplary embodiments, If SmaxG is larger, then S1 is normalized with respect to SmaxG at block 926.

According to some exemplary embodiments, If Smaxl is larger, then S1 is normalized with respect to Smaxl at block 928.

According to some exemplary embodiments, all previously obtained PSDs are re-normalized to Smaxl at block 930, and Smaxl is defined as the new SmaxG at block 932. Optionally, to re-normalize a previously normalized PSD, Sx, to the new level of Smaxl, it is sufficient to multiply Sx by SmaxG and then divide by Smaxl.

Reference is now made to FIG. 9C, depicting the use of a global value which is the largest prominence value calculated for peaks in the PSD signal during processing of a signal to detect tremor, according to some exemplary embodiments of the invention. Optionally in the process described in FIG. 9C, after calculating the peak prominences, only the largest peak prominence value is maintained, and the value of the PSD at all other frequencies is replaced with zero.

According to some exemplary embodiments, a global maximal prominence value, PmaxG, is stored in memory (initiated as equals 0), for example for a specific EMG site, at block 934. In some embodiments, each EMG site has its own global maximum, and the analysis is performed for each site separately.

According to some exemplary embodiments, an assessment system is in a standby state, waiting for an indication from a system or user, at block 936.

According to some exemplary embodiments, upon receiving an indication, for example flag for processing another input EMG signal for tremor quantification, an EMG signal is obtained at block 938. In some embodiments, the EMG signal is obtained per a new stimulation level.

According to some exemplary embodiments, the obtained signal is filtered using a high-pass filter, at block 940.

According to some exemplary embodiments, PSD is calculated, at block 942.

According to some exemplary embodiments, the peaks in the PSD of current stimulation level are detected and prominences are calculated for all the peaks, at block 944.

According to some exemplary embodiments, the maximal prominence value, Pmaxl, and the frequency at which Pmaxl appears, Fmaxl are detected, at block 946.

According to some exemplary embodiments, the PSD values are replaced with zeros, everywhere except Fmaxl, at which the value is replaced with Pmaxl, at block 948. In some embodiments, this step is performed to clarify the display of the signals, for example by maintaining only the most prominent peak and removing other components which may be distracting. Alternatively, at the locations where peaks are detected the PSD value is replaced by the peak prominences, while the PSD at frequencies where peaks aren't detected is replaced by zeros.

According to some exemplary embodiments, Pmaxl, is compared with PmaxG, at block 950.

According to some exemplary embodiments, if PmaxG is larger, then Sl is normalized with respect to PmaxG, at block 952.

According to some exemplary embodiments, If Pmaxl is larger, then Sl is normalized with respect to Pmaxl, at block 954. Additionally, all previously obtained PSDs are re-normalized to Pmaxl at block 956, and Pmaxl is defined as the new PmaxG at block 958. In some embodiments, to re-normalize a previously normalized PSD, Sx, to the new level of Pmaxl, Sx is multiplied by PmaxG and then divide by Pmaxl.

FIGS. 9D-9G describe quantitative results of an experiment comparing the 3 analysis methods, described in FIGS. 9A-9C.

Table 1 below summarizes some parameters of the recordings performed in the first experiment:

| recordingDate | brainSide | score | note | stimAmp |
|---|---|---|---|---|
| 30 Jan. 2019 | Left | 2 | NaN | 1− |
| 30 Jan. 2019 | Left | NaN | continued tremor | 250 |
| 30 Jan. 2019 | Left | 0 | Tremor Arrest | 500 |
| 30 Jan. 2019 | Left | 0 | Tremor Arrest | 1000 |

Reference is now made to FIG. 9D, depicting an example of analyzing multiple EMG signals, and displaying outputs that highlight the degree of tremor in various methods, according to some exemplary embodiments of the invention.

According to some exemplary embodiments, the three rows are respective to the three sites, sitel, site 2 and site 3, which are EMG-recording sites, or locations on the subject's body over which EMG electrodes are positioned, for example face, arm and leg. In some embodiments, each of the three columns is respective to one of the three exemplary tremor analysis methods described in previous FIGS. 9A-9C. In some embodiments, the left column, columnl, displays PSDs, in which each calculated PSD is normalized with respect to its own maximum, for example as described in FIG. 9A. The middle column, column 2, displays PSDs, that per each site, each PSD is normalized to the maximal PSD value calculated per that site, for example as described in FIG. 9B. The right column, column 3, displays PSD prominence values, in which only the maximal prominence is maintained per each calculated PSD, and each prominence value is normalized to the largest PSD prominence calculated per that site, for example as described in FIG. 9C.

According to some exemplary embodiments, the frequency range displayed is between fl and fh, in which f1 can be about 2 Hz and fh can be about 8 Hz in the case of a typical PD patient, or 4 Hz and 12 Hz respectively for a typical ET patient. Per each site, 4 increasing stimulation levels are depicted in this example, from s1 to s4, (only s1 and s4 are shown, for clarity of the figure). For example, s1 can be zero, and s4 can be 2 mA. In this example, in site 1 there is significant tremor, which is reduced as stimulation level is increased. This can be well observed in the top row and middle and right columns, wherein there is a significant peak in the PSD and its height, as well as the area below it, decreases as stimulation increases. In sites 2 & 3, no significant tremor is found.

FIG. 9E summarizes results of the 3 types of processing methods for signals received from face electrodes. FIG. 9F summarizes results of the 3 types of processing methods for signals received from arm electrodes. FIG. 9G summarizes results of the 3 types of processing methods for signals received from leg electrodes.

FIG. 9H shows an example of a high pass filter having a 1 Hz cutoff, as described in this section.

Exemplary Sensing and Quantification Strategies According to some exemplary embodiments, an assessment system comprises at least one optic sensor, for example a video camera. In some embodiments, the video camera is used to quantify one of more of tremor, dyskinesia, postural instability, gait disorder, rigidity, or muscle recruitment side effect. Optionally, a video camera is also used to detect treatment-induced gaze abnormality.

According to some exemplary embodiments, the video-based quantification of attributes is achieved by at least one of two strategies. In some embodiments, the first is segmentation of the video sequences to identify sub-structures in the images that correspond to one or more of the limbs, the head, the torso or facial structures, and per each sub-structure process the video stream to calculate various features of the movement of the sub-structure. In some embodiments, the second strategy is to process the images as a whole, or possibly define one structure as the foreground, and process the dynamic pixels related to the foreground structure, for example to calculate various features of the movement of the structure.

According to some exemplary embodiments, the segmentation, of a single foreground structure or several sub-structures is based on edge-detection or on texture identification, or on other methods such as multi-variate clustering accounting for edge features, color, texture, spatial frequency components or other features of a group of pixels in an image or in the video sequence.

According to some exemplary embodiments, for either strategy mentioned above, one or more of the following exemplary features are calculated, per a single or multiple structures: the physical range of movement (i.e. how far does the structure move), the variability of the range of movement quantified as the variance, or standard deviation, or coefficient of variance of range of movement or another measure of variability. Optionally, additional features relate to the rate of appearance of the movement, i.e. is it continuous or is it intermittent, and if intermittent then the properties of average interval between movements, median interval between movements, variability of the interval and similar characteristics may be applied.

According to some exemplary embodiments, in the temporal frequency domain, features include power in various frequency bands, such as 2-4 Hz, 4-7 Hz, 8-12 Hz, etc or any intermediate, smaller or larger range of frequencies. Additionally or alternatively, the temporal frequency domain includes the magnitude of peaks in the power spectral density (PSD), and optionally the corresponding frequencies of peaks in the PSD. In some embodiments, the total-harmonic-distortion (THD) relating to a specific fundamental frequency is calculated as

$$T H D ( {f}_{t} ) = {\sum }_{m} PSD ( {mf}_{t} ) / P S D ( {f}_{t} ) ,$$

which measures the degree of deviation of a rhythmic movement from a sinusoidal oscillation.

According to some exemplary embodiments, when employing the first strategy of sub-structure segmentation, pairwise calculations are performed between pairs of sub-structures, such as correlation or cross-correlation, phase delays and cross-coherence calculations. In some embodiments, higher-order calculations, quantifying the relations between movements of 3 substructures or more, can also be carried out.

According to some exemplary embodiments, tremor detection is based on detecting rhythmic movement of the limbs, the head or in the facial muscles. In some embodiments, the highly rhythmic movement is identified by a large peak in the frequency-domain, for example in the 3-7 Hz range or any intermediate, smaller or larger range of frequencies. In some embodiments, the large peak at the fundamental tremor frequency fₜ in the 3-7 Hz range, is accompanied by peaks at the harmonic frequencies that are the products of m×fₜ, m=2, 3, etc. In some embodiments, in the case of tremor, high cross-correlation and cross-coherence values are expected as the rhythmic movement that often occurs in more than one limb, often has the same fundamental frequency, and is likely to appear and disappear in synchrony over the various body parts.

According to some exemplary embodiments, tremor is identified and quantified when the patient is instructed to be at rest, and not performing voluntary movements, further highlighting the non-voluntary movement associated with tremor. This is also true for quantification of dyskinesia and of motor recruitment side effects and gaze abnormality.

According to some exemplary embodiments, quantification of dyskinesia employs the similar two strategies described above, of processing video sequences of pixels in a single foreground structure or multiple sub-structures. In some embodiments, for example in the case of dyskinesia, cross correlations and/or cross-coherence is expected to be lower than in patients exhibiting tremor. In addition, the movement is typically less rhythmic, and the frequency-domain peak is expected to be lower, if it has any observable value at all. In some embodiments, the maximum range of movement is expected to be larger than found during tremor.

According to some exemplary embodiments, identification and quantification of motor side effects require sensitivity to muscle contractions in the face, arms and/or legs. In some embodiments, an occurring contraction would be visible as a pulling on facial muscles and causing movement of the mouth corners, or near the eyes. These are often isolated phenomena, in the sense that a treatment-induced muscle contraction in one part of the body would appear without a similar contraction in another part of the body. This limitation of the phenomena in space makes them more difficult to even detect, as global features calculated from the entire image or structure would “average out” the local effect of muscle contraction. In some embodiments, quantification of muscle contraction side effects require calculating features from smaller sub-structures in the video sequence.

According to some exemplary embodiments, postural stability is quantified when the subject is standing up and/or walking. In some embodiments, gait characteristics are visible when the subject is walking. In some embodiments, quantification of gait and/or postural stability requires a different setup and camera configuration, than the setup and camera configuration required for example to detect and quantify the local manifestations of treatment-induced motor recruitment. While the latter setup and configuration is aimed to enable detection of small changes in a small region of the face, the former aims to capture images of the whole body or large portions of the body, either static or optionally walking over several cycles of movement, and thus the required setup and configuration may be inherently different. Thus, in some embodiments, to achieve quantification of the treatment-induced motor recruitment side effect and one of the symptoms of postural instability and gate disorder, at least one additional camera is required, and at least one additional setting up stage in the preparation process, or alternatively the camera setup must be updated as needed during the recording session.

According to some exemplary embodiments, quantification of rigidity via analysis of a video sequence requires a limb of the patient not to be static. In some embodiments, the limb is moved by a second person, or by the subject themselves, and the analysis is focused on quantifying how easy the passive movement is, or how the limb continues in passive movement after the maneuver ends.

According to some exemplary embodiments, in order to optimize the performance of the video camera, a specific background is employed. In some embodiments, a clinician or the subject are instructed to select a location on premises, be it in the clinic or the house or elsewhere, in which the background complies best with predefined requirements. Additionally in this case, the system provides an indication about the quality of the compliance of the background with requirements, either by a score, e.g. of 1-10, or by a binary compliant/non-compliant indication. Alternatively, a specific background sheet or cloth is used by the clinician or the subject. Optionally, the sheet or cloth is clean of any lines or texture variations. Alternatively, the sheet or cloth has a background clean of lines and texture variations, and a foreground with lines at regular intervals or some predefined divisions or a selected pattern.

According to some exemplary embodiments, the clinician or subject or someone assisting the subject at a home environment is instructed to locate the cloth at a specific distance behind the subject while the video is being captured. Additionally or alternatively, instructions are given as to a specific angle in which the background is located with respect to the subject and the camera. This kind of background may assist in calibrating the video processing algorithms by assessing the distance to the subject, or the angle to the subject. Alternatively or additionally, the background improves the video quantification performance by enhancing the contrast between the subject and the image background.

### Exemplary Condition Assessment Using EMG

According to some exemplary embodiments, EMG recordings are used to assess the condition of a subject before, during and following a brain stimulation treatment, for example DBS. In some embodiments, the EMG recordings are used to quantify at least one symptom of a neurological disease and/or at least one side effect of the brain stimulation treatment.

According to some exemplary embodiments, EMG electrodes are applied over pre-determined muscles of a patient, for example a patient of a neurological disease. In some embodiments, the electrodes are then connected to an assessment system or an assessment device.

According to some exemplary embodiments, signals are recorded during a baseline condition, for example when the patient is at rest or when the treatment is stopped. Alternatively, the signals are recorded at a selected time period, and are then termed as reference signals.

According to some exemplary embodiments, at least one parameter related to the treatment is changed, for example stimulation amplitude, stimulation frequency, stimulation duration, number of stimulation pulses in a train of pulses, number of trains, and/or duration of each train. In some embodiments, the at least one parameter comprises position of at least one stimulation electrode along a lead, number of stimulation electrodes, insertion depth of the lead, and/or position of the at least one electrode within the brain.

According to some exemplary embodiments, the signals are recorded while the patient is at rest. In some embodiments, the patient is then instructed to perform a task, and additional signals are recorded during and following the task performance.

According to some exemplary embodiments, the signals, for example baseline signals, signals recorded in rest and signals recorded and task-related signals. In some embodiments, the signals are pre-processed prior to feature calculation. In some embodiments, features are calculated from the pre-processed signals.

According to some exemplary embodiments, an index for one or more of symptoms, signs or side effects is calculated. In some embodiments, the index is calculated as at least one linear combination or at least one non-linear combination of the calculated features.

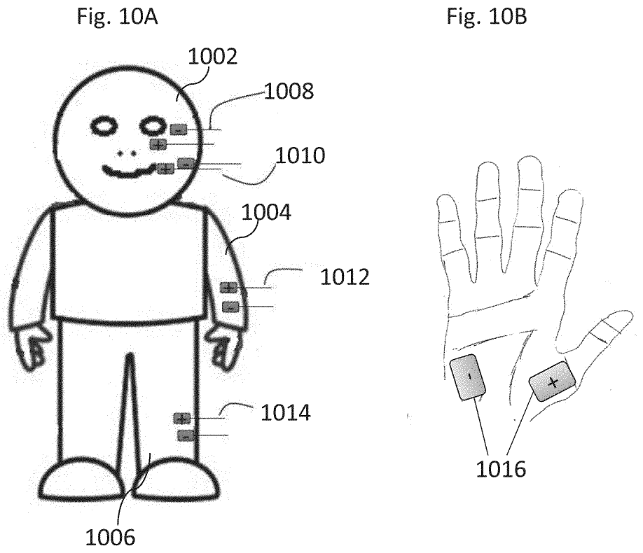

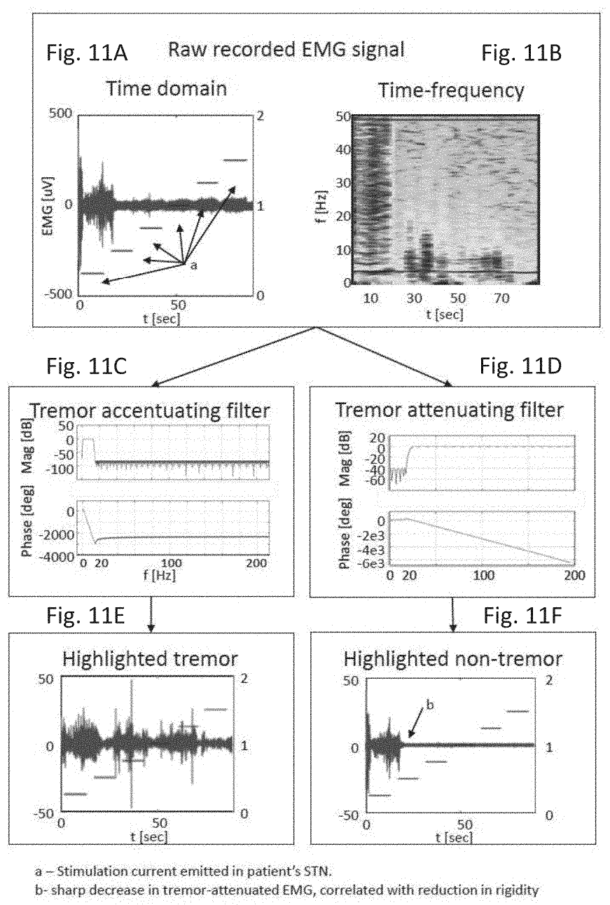

Reference is now made to FIGS. 10A and 10B, depicting locations for placement of EMG electrode pairs, according to some exemplary embodiments of the invention.

According to some exemplary embodiments, one or more EMG electrode pairs are placed on the face 1002, at least one hand 1004 of the patient, and at least one leg 1006 of the patient. In some embodiments, at least one EMG electrode is placed at location 1008 on the face, for example to record signals from the Orbicularis Oculi muscle. In some embodiments, the at least one EMG electrode is placed at location 1010 on the face, for example to record signals from a mixture of two or more of the Zygomaticus muscle, the Masseter muscle, the Buccinator muscle, and the Risorius muscle.

According to some exemplary embodiments, at least one EMG electrode is placed at location 1012 on the hand 1004, for example to record signals from the Extensor Carpi Radialis muscle and/or the Flexor Carpi Radialis muscle.

According to some exemplary embodiments, for example as shown in FIG. 10B, at least one EMG electrode is positioned at location 1016 on the hand, for example to record potential difference between the Opponens pollicis and mixture of Opponens digiti minimi and Flexor digiti minimi brevis.

According to some exemplary embodiments, for example as shown in FIGS. 11A-11F, and as described in FIG. 8A time domain and/or a time-frequency representations are generated from a raw recorded EMG signal, followed by using tremor accentuating and tremor attenuating filters to highlight tremor and non-tremor related signals respectively.

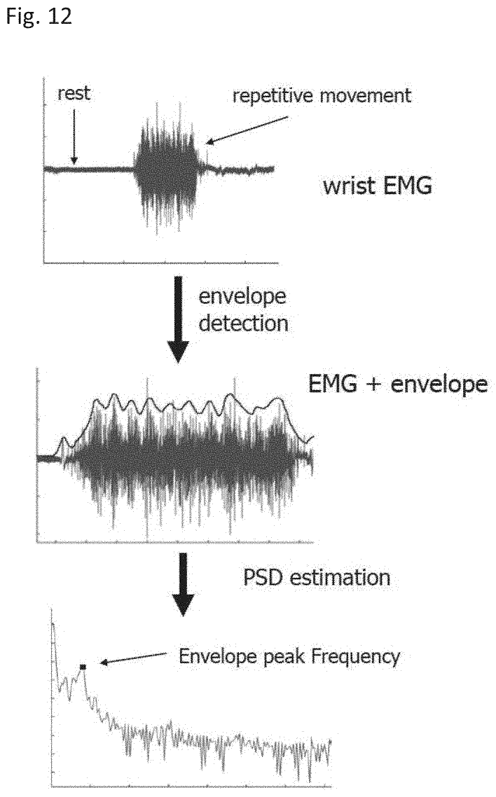

According to some exemplary embodiments, for example as shown in FIG. 12, envelope detection is performed on a wrist EMG signal, followed by PSD estimation, for example to identify the envelope peak frequency.

Exemplary Rigidity Assessment According to some exemplary embodiments, the assessment system is configured to assess rigidity, by a sensor which is a rigidity-measuring device aimed at quantifying the mechanical properties of a limb being rotated around a joint, such as an arm, wrist or ankle, as a proxy for the clinical symptom of muscle rigidity. In some embodiments, the devices, for example the devices described in “A portable system for quantitative assessment of parkinsonian rigidity” by Houde Dai, Bernward Otten, Jan Hinnerk Mehrkens, L. T. D'Angelo, 35th Annual International Conference of the IEEE EMBS, 2013, and “Quantification of the UPDRS Rigidity Scale” by Susan K. Patrick, Allen A. Denington, Michel J. A. Gauthier, Deborah M. Gillard, and Arthur Prochazka IEEE TRANSACTIONS ON NEURAL SYSTEMS AND REHABILITATION ENGINEERING, VOL. 9, NO. 1, MARCH 2001, utilize Newton's second law of motion in its angular application: T=Iα, wherein T is the torque applied to the limb, I is the moment of inertia and a is the angular acceleration. In some embodiments, the resistance to rotation, embodied by I in the equation, depends on the passive mechanical properties of the limb, as well as the reactive mechanical properties of the muscles, which are influenced by the presence of the rigidity symptom.

According to some exemplary embodiments, a rigidity-measuring device containing multiple sensors is shaped and sized to be attached to a tested limb. In some embodiments, the rigidity-measuring device is shaped as a cuff, that is positioned over the arm and is either elastic and conforms tightly to the limb or it has some specific tightening-loosening feature such as a hook and loop fastener. In some embodiments, at least some of the sensors are sensitive to changes in position, such as accelerometers, gyroscopes and magnetometers.

According to some exemplary embodiments, these sensors are found in single packages termed inertial measurement unit. In some embodiments, each property (acceleration, angular velocity or magnetic field) is measured in 3-axes, as the motion of the limb in a real-world setting occurs in 3 axes. In some embodiments, the aim of utilizing these Inertial Measurement Unit (IMU) sensors (whether or not packaged in an IMU), is to accurately record the position of a location on the limb, despite intrinsic errors in each of the sensors. In some embodiments, a combination of accelerometer and gyroscope is sufficient to obtain a reasonably accurate position. Optionally, readings of a magnetic sensor sensitive to horizontal motions are added to the accelerometer readings that is mostly sensitive to the vertically oriented force of gravity.

According to some exemplary embodiments, the limb is moved controllably and automatically or semi-automatically, for example by a mechanical device, in which the applied force is measured intrinsically. Alternatively, the limb is moved by a second person, or a device in which the force is not directly controlled (such as continuous passive motion device) and then the force is measured by one or two force meters attached to the rigidity-measuring device, sensitive to the force applied to it by the second person or machine. In some embodiments, to convert the force measurements to torque, the distance from the point of force application to the joint must be measured or estimated, as T=Fl, in which F is the net applied force and l is the torque arm.

According to some exemplary embodiments, the mechanical measurements are then used to calculate mechanical parameters of the limb, via the equation T=c|ω|+d|θ|+e, in which ω and θ are the angular velocity and the limb angle respectively, calculated from the IMU sensor readings, c and d are the elastic stiffness and viscosity of the limb, and e is a constant error. In some embodiments, c, d and e are scalar parameters, while T, ω and α are continuous variables calculated from the readings. Thus, the elastic stiffness and viscosity are estimated from the set of readings, via any fitting tool such as linear regression. In some embodiments, another relevant index is mechanical impedance, defined as Z=c+d2πf, wherein f is the frequency of repetition of the movement of the limb. In some embodiments, the parameters c, d and Z, are correlated to varying degrees with the presence of rigidity and its severity.

According to some exemplary embodiments, the output of this rigidity-measuring module is used by itself, or coupled with EMG measurements, as described herein, for example to obtain a more robust quantification of rigidity.

According to some exemplary embodiments, a process of obtaining a rigidity measurement comprising the following steps:
a. Attach the rigidity measuring device to the patient arm according to specific instructions (e.g. arrow indication on device pointing towards elbow or away from elbow).
b. Optionally, measure, estimate or otherwise obtain an estimation of the length l between the center of the rigidity measuring device and the patient's elbow or more accurately the elbow's fulcrum. This length, l, is the torque arm length and is required to convert the measurements of forces to measurements of torque. The estimation may not necessarily require performing a measurement, for example it may be possible to estimate the length from other properties of the patient, such as height, weight, age, etc.
c. Optionally, start with tested arm resting and horizontal (about 1-2 seconds), and no force (or small force) applied to grip the device by the operator. This allows an initial period in the recording that is in a known position, improving the estimation of the initial orientation (based on gravity effect measure by accelerometer), and the location of the periodic manipulation in the signals.
d. Optionally hold the subject's elbow—on the tested side—with one hand.

Alternatively, the elbow is fixed.
e. With the 2^{nd} hand hold the patient's tested arm by the rigidity device, in the location marked on the device to ensure the applied force is measured by the force sensors. Alternatively, holding can be done by a mechanical device, for example a lever or a robotic arm instead of the human holding the subject's hand.
f. Perform repetitive vertical or horizontal flexion and extension of the patient's arm, about the axis of the elbow. In some embodiments, force is applied, that causes rotation around the elbow joint, or optionally around the wrist joint. In some embodiments, the flexion/extension and the repetitions are optional.
g. Optionally, complete the process by returning to a resting horizontal position (about 1-2 seconds).

Calculation procedure
h. Determine the time intervals of the initial rest and the repetitive manipulation, optionally by:
   i. Generate a 1-d signal from the 3 gyroscope signals that are measured one for each axis. This can be done by:
      1. Taking at each time sample the total energy of the 3 signals
      2. Selecting the signal from the axis that changes most during the manipulation. This can be achieved robustly by calculating the inter-quartile range (IQR) for each axis signal, and selecting the axis with the highest IQR. IQR is more robust to noise and outliers than simple range (max(x)-min(x)).
   ii. Transform the gyroscope signals to a representation that emphasizes total movement energy. Example:
      1. smooth the gyro signal with moving window or low-pass filter
      2. take the square (x²)
   iii. Process the obtained result to locate manipulation
      1. Establish the baseline mean and standard deviation (STD), or median and MAD.
      2. Beginning at the beginning of the signal (time=0), find time points in the signal that deviate from mean+STD by more than a threshold.
      3. Check for a minimum number of consecutive threshold-crossing samples, to make sure the threshold crossing represents changing from “rest” to “manipulation” states, and not a random noise or artifact result.
      4. Perform a similar check to steps (2) & (3), beginning from the end of the signal and going backwards, to determine the transition back from manipulation to the “rest” state.

In some embodiments, if force is applied by mechanical device—this determining comprises reading output from the device when it applied force to the patient. Repetitive manipulation is optional as above.
i. In some embodiments, determine initial orientation of the device during the detect “rest state”. This is achieved by analyzing the accelerometer signals, that at rest are mostly influenced by the force of gravity. The static, offset value measured by the 3 accelerometer axis at rest are the 3 components (x, y, z) of the force of gravity, enabling to determine the orientation of the device up to rotation about the axis of the direction of gravity. By knowing the orientation of device attachment to arm (see 1.a.), the orientation in 3-d may be completely known.
j. Apply the orientation detection algorithm, such as is cited by Dai et al., to detect the angle between the arm and the horizon during the manipulation. Use the baseline orientation in the previous step to optionally correct the calculated angle to correspond to the angle between the elbow and the horizon. This may require to invert the sign of the angle (from +to −), or to apply a pi/2 rotation. In some embodiments, application of the orientation detection algorithm is required when a human moves the patient arm, and there is no other sensor for the angle (such as a goniometer). In some embodiments, if arm movements are performed by mechanical device application of the orientation detection algorithm is not required.
k. Use the calculated angle θ, its time derivative w and the torque T=Fl, to perform the fitting or regression described above to extract elastic modulus and viscosity. In some embodiments, the angle theta is directly measured, without calculation.

### Exemplary EMG Rigidity Analysis

According to some exemplary embodiments, rigidity is estimated from EMG recordings of a patient at rest. In some embodiments, rigidity related signals are separated from tremor-related signals, prior to rigidity analysis.

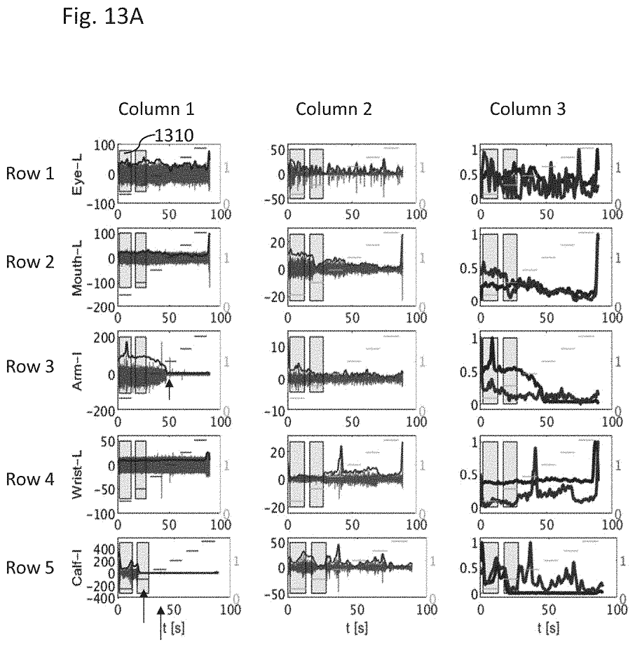

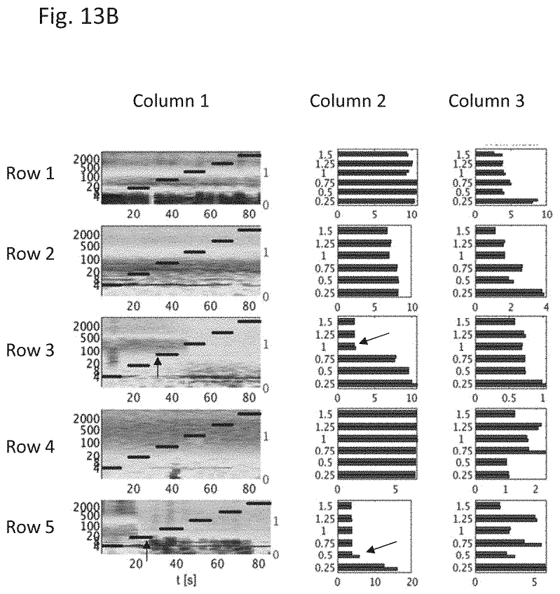

Reference is now made to FIGS. 13A and 13B, depicting the results of an EMG rigidity analysis.

In the experiment and in some embodiments, tremor analysis is performed by first decimate the acquired signal, for example decimate to 440 Hz. Following decimation the signal is passed through a band pass filter, for example a band pass filter of 2-13 Hz. In the experiment and in some embodiments, a Gauss-kernel moving window RMS is calculated. Analysis results are displayed, for example as bars, by showing mean and/or median of RMS during stimulation.

In the experiment and in some embodiments, rigidity analysis is performed by passing the acquired signal through a LPF filter, for example a LPF filter with a cutoff at 2000 Hz, followed by a HPF filter, for example a HPF filter with a cutoff at 20 Hz. In the experiment and in some embodiments, a Gauss-kernel moving window RMS, which is a method to calculate average RMS localized around a specific time point, is calculated. Analysis results are displayed, for example as bars, by showing mean and/or median of RMS during stimulation. In the experiments, it was found that the EMG signal recorded while the patient is at rest, after filtering out the effects of tremor, is correlated with the clinical symptom of rigidity. In some embodiments, and in the experiment reduction in the power in the frequency band of 20-2000 Hz, quantified as detailed above, is found to occur at the same treatment level at which reduction in rigidity was found by an expert's clinical assessment.

In FIGS. 13A and 13B, column 1 represents Rigidity-processed signal+moving-window RMS of the signal; column 2 represents Tremor-processed signal+moving-window RMS of the signal; column 3 represents moving-window RMS of rigidity- and tremor-processed signals; column 4 represents time-frequency representation of the raw EMG signal; column 5 represents rigidity indices per stimulation level, calculated by taking mean or median values of the rigidity-processed signal at each stimulation level; column 6 represents tremor indices per stimulation level, calculated by taking mean or median values of the rigidity-processed signal at each stimulation level.

In FIGS. 13A and 13B Columns 1-3, x-scale is time [sec], y-scale is in micro-Volts; column 4-, x-scale is time in secs, y-scale is frequency in [Hz] (logarithmically scaled); columns 5 & 6—x-scale is the size of the rigidity or tremor index, y-scale is the stimulation current applied in milli-Amperes.

In FIGS. 13A and 13B, row 1 represents an EMG signal measured next to the subject's eye; row 2 represents an EMG signal measured next to the subject's mouth; row 3 represents an EMG signal measured from the subject's arm; row 4 represents an EMG signal measured from the subject's wrist; row 5 represents an EMG signal measured from the subject's leg. In FIG. 13B at column 2, rows 3 and 5, the arrows point to a stimulation level in which the rigidity index is reduced in the arm and in the leg respectively.

In FIG. 13A, column 1, the 2 semi-transparent rectangles, for example semi-transparent rectangle 1310 depict the intervals in time, in which delivered treatment levels were clinically found to reduce the patient's rigidity. The arrows in rows 3 and 5 of column 1 point to time points at which the EMG signal, processed for rigidity analysis as described above, undergoes a significant reduction. In row 5, the arrow and the semi-transparent rectangle coincide, indicating that the change in the signal is in timely agreement with the clinical assessment. In row 3 the arrow and the semi-transparent rectangle do not exactly coincide, yet this may be due to the methodology of the experiment, in which the clinical assessment occurs during one period of stimulation, and the EMG-based assessment occurs in a second period that occurs a few minutes later. Thus, the overall stimulation regime isn't exactly the same, and some discrepancies may occur. As such, the result displayed at row 3 is potentially also an example of correlation between reduction in clinical assessment of rigidity and the rigidity-processed signal.

### Exemplary Speech and Dysarthria Assessment

According to some exemplary embodiments, speech and/or dysarthria assessment are performed based on signals recorded by at least one audio sensor. In some embodiments, audio sensors capture the subject's speech articulation, and the signals are processed for example, in order to quantify at least one of two attributes: the amplitude of speech and the clarity of speech.

According to some exemplary embodiments, to quantify dysarthria, the articulation at a specific treatment level or using a selected set of treatment parameter values, is compared with prior articulation scores of the same patient at time points in the past, or to articulation scores of a population of other subjects. In some scenarios, such as intra-operatively or in post-operative tuning of DBS or pump therapy levels, the articulation at a specific therapy level is compared to the articulation of the same patient in the absence of therapy, or at a baseline level of therapy, optionally within the time frame of the tuning session (up to about an hour, or up to about 20 minutes or any shorter or longer time period). In some embodiments, the processing required to perform this comparison is based on general signal processing methods, such as filtering, envelope detection and spectral estimation, and optionally using methods from the more focal fields of speech recognition and speaker recognition.

According to some exemplary embodiments, speech recognitions techniques are aimed at translating acquired articulated sounds to specific words in one or more languages, for example using methods described in “Speech recognition with deep recurrent neural network” published by Graves et al. in 2013 IEEE International Conference on Acoustics, Speech and Signal Processing. In some embodiments, a flow of such techniques includes acquiring the articulated sound (sensing the physical sound wave, and digitizing it), preprocessing it by optionally filtering followed by calculating a set of representing features. In some embodiments, the set of representing features include time-frequency representations of the sound (optionally via mel-frequency Cepstrum (MFC), but Short-Time Fourier Transform, Wavelet Transform, and other methods are applicable), which are then optionally fed to a machine-learning classifier that fits the highest likelihood letter to signal time bins. According to some exemplary embodiments, machine learning classifiers are trained on a data set that includes multiple digitized utterances and their textual translation. An example to commonly used classifiers are deep recurrent neural networks (RNNs), hidden Markov Models (HMMs) and combinations of HMMs and various types of neural networks.

According to some exemplary embodiments, speaker recognition techniques are aimed at identifying the speaker's identity, either based on specific spoken text, or not. In some embodiments, a similar general flow of acquisition, preprocessing, feature extraction and classification applies, and the difference from speech recognition is that the classifier is trained to minimize errors in recognizing the speaker correctly, and the features are selected to minimize this classifying error. According to some exemplary embodiments, the MFC coefficients are used as features for speaker recognition, as well as mean-subtracted cepstra, and the 1^{st} and 2^{nd} derivatives of these features (knowns as deltas). Alternatively, other features that are used for speech recognition and speaker recognition include one or more of frequency domain linear prediction (FDLP), mean Hilbert envelope coefficients (MHECs) and power-normalized cepstral coefficients (PNCCs). In some embodiments, the classifier is based on a non-parametric model, such as a Dynamic Time Warp (DTW) or nearest-neighbors, or on parametric models such as vector quantization, Gaussian mixture models, HMMs and support vector machines (SVMs).

According to some exemplary embodiments, in the specific sub-branch of speaker verification, the aim is to decide whether the likelihood of the speaker being a specific person that the system trained on, is significantly larger than the probability that it is any other speaker in the population. In some embodiments, in this case, a similarity index is calculated from the set of features to represent the degree of similarity between the tested vocalization and the voice in the training database, and comparing this to a threshold representing how “significant” is defined in the specific system.

According to some exemplary embodiments, in the setting of identifying disease-related or treatment-induced changes in the articulation, it is not the objective to identify the speaker, or the spoken text, yet there are several paths in which these techniques are utilized. One way is to compare the success rate in speech recognition of the same articulated text at any treatment configuration, and baseline. In some embodiments, an increase in the recognition success rate is correlated with decrease in the deteriorating speech symptoms, while a decrease in the success rate indicates dysarthria side effect. In some embodiments, dysarthria is identified by utilizing a speaker verification technique, when the vocalization of a patient receiving DBS treatment is not recognized as belonging to the same patient recorded at baseline treatment levels.

According to some exemplary embodiments, a second way to utilize these techniques is not to rely on the final output, that is correctly identified speech or verified speaker, but to use one the interim calculations. For example, the speaker verification similarity is used by itself, regardless of the result of a comparison with a threshold. In some embodiments, the similarity index is tracked during tuning of the treatment, as well as its variability and tendency to change, both spontaneously and in relation with the treatment tuning. In some embodiments, when at a certain treatment level the similarity index changes to an extent that is significantly different from the established trend, it is indicative of dysarthria. The same holds for likelihoods calculated in a speech recognition process, that is required to decide which letter or word (or letters or words) is most likely being uttered. Even though the final classification result is not changed, a reduction in the likelihood of the correct number that exceeds variability due to spontaneous differences between repetitions of the same articulation, can be indicative of dysarthria.

According to some exemplary embodiments, primitive features used as input to the classifiers are fed into a new classifier, trained specifically to detect dysarthria. In some embodiments, both primitive features, and more downstream results of processing such as similarity index or uttered letter probability, are fed into a new classifier that is trained to detect dysarthria.

According to some exemplary embodiments, to train a dysarthria classifier, first a database of vocalizations and their related tags is constructed, including speaker identification (speaker #001, #002, etc. . . . ), and speaker condition—(normal vocalization, vocalization degraded due to disease symptoms, or treatment-induced dysarthria). In some embodiments, the classifier is trained on this data through one of the many machine learning supervised classification methods (SVM, decision tree, random forest, Naïve Bayes, HMM, artificial neural networks etc. . . . ), for example to minimize its prediction errors. In some embodiments, two settings are possible—first, in which the classifier is trained only to detect or reject the dysarthria condition, and the second in which it is required to distinguish between normal speech, disease-related abnormal speech, or treatment-induced dysarthria. In some embodiments, while the second option is more informative, and is used in more applications, such as patient diagnosis or assessment of patients before advanced treatment is employed, it is more difficult to train, would require a larger database and may result in larger error rates.

An example of using basic features to separate between recordings of patients with dysarthria and patients without dysarthria is described below. The described steps allow, for example, separation between groups of stimulation with dysarthria and stimulation without dysarthria. Thus, in some embodiments, given a new recording, it can be classified to one of these groups and dysarthria can be detected if it exists.

According to some exemplary embodiments, capability for detecting Dysarthria in human speech are prepared, for example, by recording vocal articulation data from multiple subjects, in which each recording is labeled as “Dysarthria” or “Not-Dysarthria”.

According to some exemplary embodiments, an algorithm for detecting the dysarthria in the recordings is constructed by calculating features in the recorded signals.

According to some exemplary embodiments, a model in which the various features are combined to a single number via a mathematical relationship, for example a linear combination model, or a non-linear model such as a Generalized Linear Model (GLM) is defined. In some embodiments, the model has several coefficients that are unknown at the outset.

According to some exemplary embodiments, the model coefficients, or the exact combination of the calculated features per each recording, that yields an optimal separation between the groups of recordings labeled as “Dysarthria” and those labeled “Non-Dysarthria” are inferred (or learned as in “machine-learning”).

According to some exemplary embodiments, the generated algorithm is used to detect the dysarthria side effect, in a patient being assessed. In some embodiments, vocal articulation data is recorded from the patient. In some embodiments, the algorithm is applied to the recorded data to examine whether Dysarthria is present in the recording or not.

According to some exemplary embodiments, the binary decision described in previously (for example True/False, Dysarthria/Not-Dysarthria) is followed by a quantification of the severity of the side effect in the examined patient. In some embodiments, this is performed for example by measuring a distance between the point representing the examined patient in the recording feature-space and the line (or curve, or plane, or other geometric entity) that represents the threshold between the two groups. In some embodiments, the larger the distance in feature-space, the higher the severity score assigned to the patient's dysarthria.

In some embodiments, over longer time frames than the DBS surgery itself or a DBS programing session, the voice of a patient not receiving advanced treatment is repeatedly recorded and analyzed, and changes in the recognition outcomes indicate a significant worsening of articulation due to disease progression. Such an event may lead to presentation of an indication to the system user, be that a caregiver, movement disorders specialist or a non-professional such as a family member or the patient themselves, suggesting further consultation and possible adjustment of treatment.

### Exemplary Gaze Disorder Assessment

According to some exemplary embodiments, the assessment system is used to detect gaze abnormalities, for example treatment-induced gaze abnormalities. In some embodiments, at least one sensor of the system is configured to track the movement range of the patient's eyes, and to detect treatment-induced gaze abnormalities. In a DBS setting a common gaze abnormality is a limitation in the movement of one of the eyeballs that should be tested during an active task of moving the eyes to each side as far as possible. In some embodiments, to achieve this goal, the sensors engaged can include eye-tracking devices for example as described in www(dot)cs(dot)cmu(dot)edu/˜ltrutoiu/pdfs/ISWC_2016_trutoiu(dot)pdf. In some embodiments, the eye-tracking devices include video eye-trackers, for example video eye-trackers based on infrared (IR) light directed at the eyes, locating the identified corneal reflection (CR, 1^{st} Purkinje image) and using the vector between the pupil center, or the iris center, and the CR to infer the direction of gaze. In some embodiments, the video eye trackers do not make use of IR light, but are based on image processing to locate the eyes and the pupil/iris in an image and then calculate the gaze direction from the pupil position and/or visible shape. In some embodiments, a calibration step is performed for any of these methods, in which the patient performs a set of pre-defined eye movements at a baseline treatment level.

According to some exemplary embodiments, a technique based on recording the electrooculogram (EOG), i.e. the voltage recorded between two or more surface electrodes on the skin around the eye as is influenced by the dipole between the negatively charged retina, and the cornea, is used. As the eyeball turns, the dipole rotates and the voltage between a pair of surface electrodes changes accordingly, for example becoming negative or positive according to the direction of the eye movement. In some embodiments, this method allows to measure for each eye the maximal voltage deflection obtained when moving the eye to each extreme position (left, right, up, down) at baseline, and then compare the EOG voltage deflection during treatment. In some embodiments, when the voltage deflection for one eye is similar to the baseline deflection, while for the second eye it fails to reach the baseline deflection, this is indicative of treatment-induced gaze abnormality.

According to some exemplary embodiments, the eye position is tracked by attaching a soft contact lens with embedded mirrors or magnetic field sensors, and following the position of these using a camera or magnetic coils.

### Exemplary Gaze Analysis

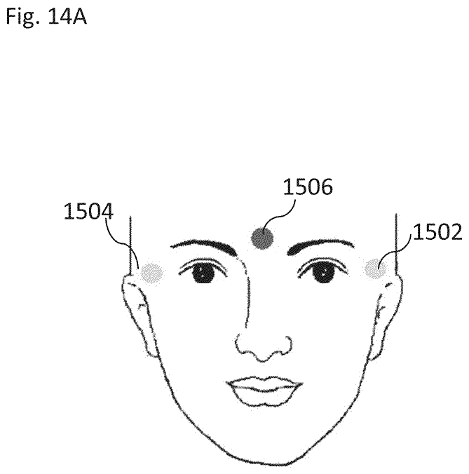

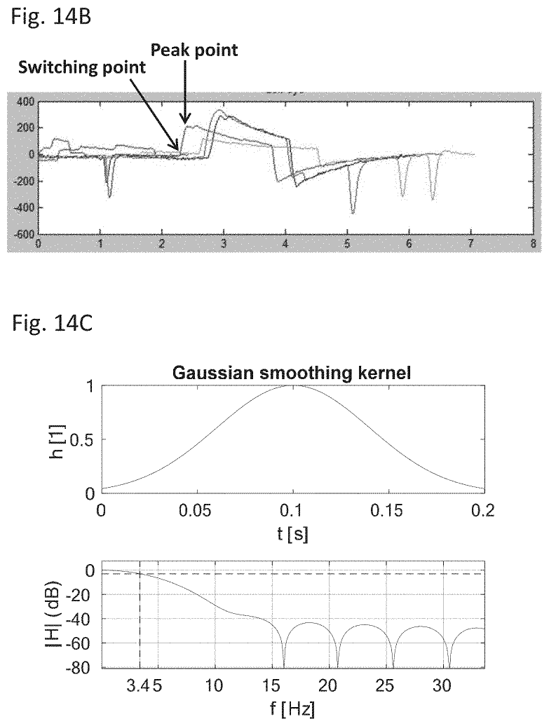

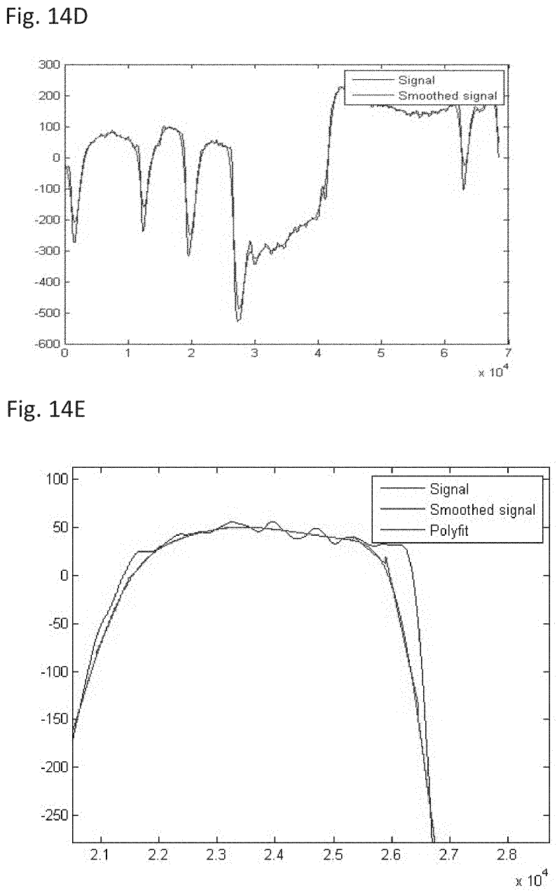

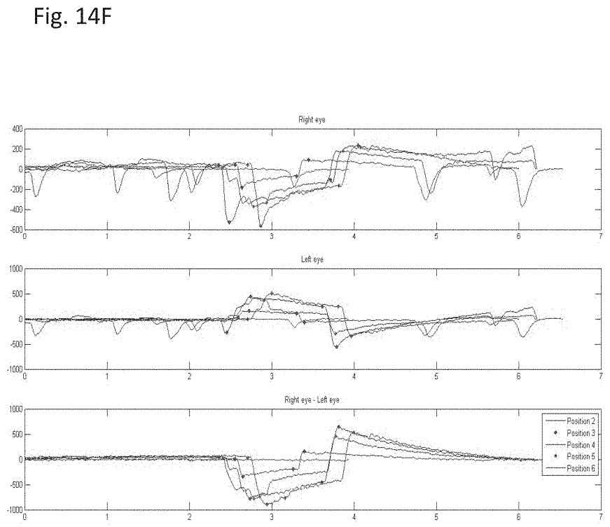

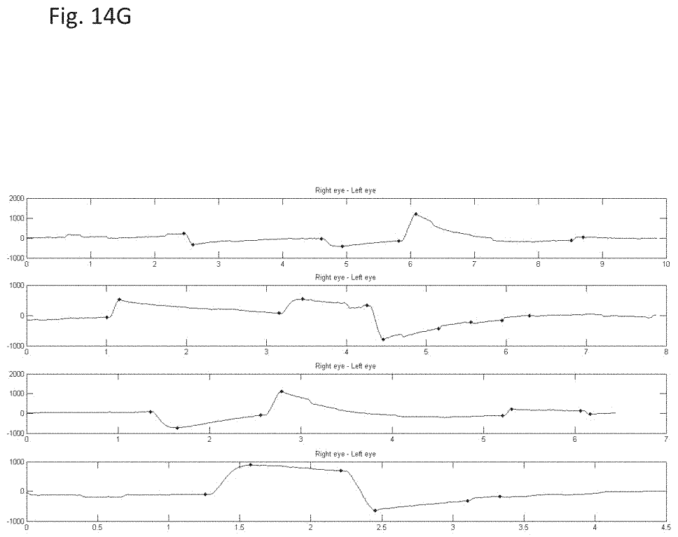

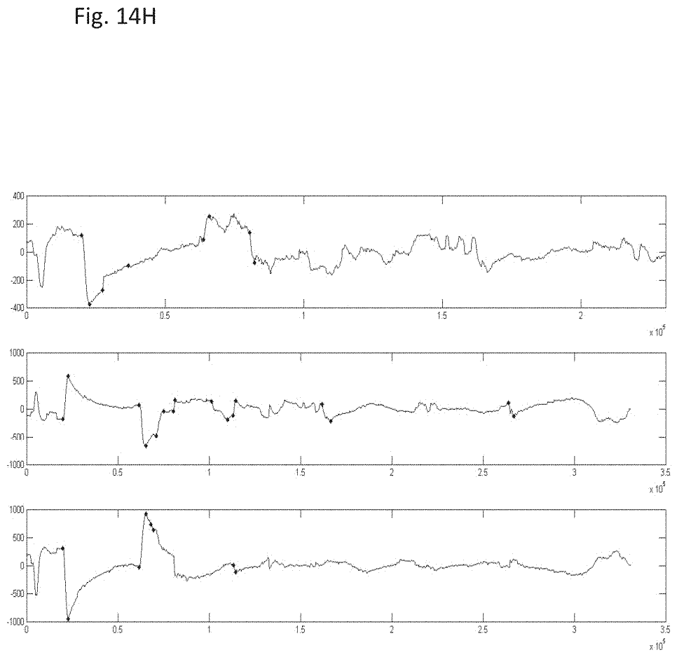

According to some exemplary embodiments, in order to measure signals for assessment of gaze, at least two electrodes, for example EMG electrodes, each electrode is placed on one side of the face near an eye, for example as shown in FIG. 14A. In some embodiments, the electrodes are positioned at a location close to the eye, for example locations 1502 and 1504, for example to measure activity of muscles related to eye movements. In some embodiments, at least one reference is positioned on the face or at a different location on the body. In some embodiments, the at least one reference electrode is positioned over frontal bone just above the Nasion 1506.

According to some exemplary embodiments, in order to detect eye movement, potential differences between the voltage in steady state and the voltage at a peak point pf a signal are measured. In some embodiments, to measure these points values, an algorithm to detect the starting eye movement point (switching point) value and its related peak value is used, for example as shown in FIG. 14B. In some embodiments, for gaze palsy detection, a step value measured at baseline (without stimulation) is compared with the step value measured at stimulation. In some embodiments, an indication regarding gaze palsy is received when the difference is higher than a predetermined tolerance.

An alternative approach is to establish a baseline step size for each side (eye), and per stimulation level calculate current step sizes per each eye and compare to the baseline. Significant deviation of only one eye from baseline, while the value for the 2^{nd} eye is according to the baseline, indicates gaze palsy.

FIGS. 14C-14I describe the results of an exemplary gaze analysis using a signal processing method for detection of gaze.

In the gaze analysis and in some embodiments, the received data is passed through a bandpass filter (for example Butterworth order=2, cutoff=0.1-20 Hz). Then, in some embodiments, the signal is smoothened by convolving the signal with Gaussian, for example as shown in FIG. 14C.

In the gaze analysis and in some embodiments, following smoothening, the signal is divided into segments with the same size, for example segments with duration of 50 msec. Then, in some embodiments, a polyfit function is applied on each segment, for example to receive the closest trend line, meaning fitting a linear approximation for each segment.

In the analysis and in some embodiments, switching points are then identified, for example by checking a line slope of the linear approximation of each segment which is larger than a predetermined value. Following switching points detection, the peak values are then measured.

FIG. 14D depicts the results of a signal smoothening process. FIG. 14E shows the results of a polyfit function application on selected segments.

In the figures showing the results, the algorithm was set to ignore a selected time duration between switching point and peak point greater than 1 second, a step value smaller than 35 uV, and a peak width at half max which is less than 0.4 seconds, which optionally indicates blinking. FIG. 14F describes results of the signal processing method showing one side movement.

In the analysis and in some embodiments, in order to detect movement of the eye pupil to the side in two or more steps, we added the values of the steps. This allows to get the full step value. i.e. when the patient moves the eye pupil in one step. The algorithm, in some embodiments, adds the values of the adjacent steps with the same slope direction.

FIG. 14G describes results of the signal processing method showing eye movement with 2 steps. FIG. 14H describes results of the signal processing method using data from a real surgery. FIG. 14I describes the full range of the results using the signal processing method.

### Exemplary Internal Capsular Recruitment Assessment

According to some exemplary embodiments, an algorithm is used to detect motor movement which caused by internal capsular recruitment, for example as a result of electrical stimulation. In some embodiments, the algorithm is used to detect recruitment (artificial activation due to electrical current leakage) of the facial muscles, which is a frequent side-effect encountered in DBS during surgery or during programing of the IPG. Alternatively or additionally, the algorithm is used to detect recruitment of muscles in the upper limbs or the lower limbs, also a side effect of DBS.

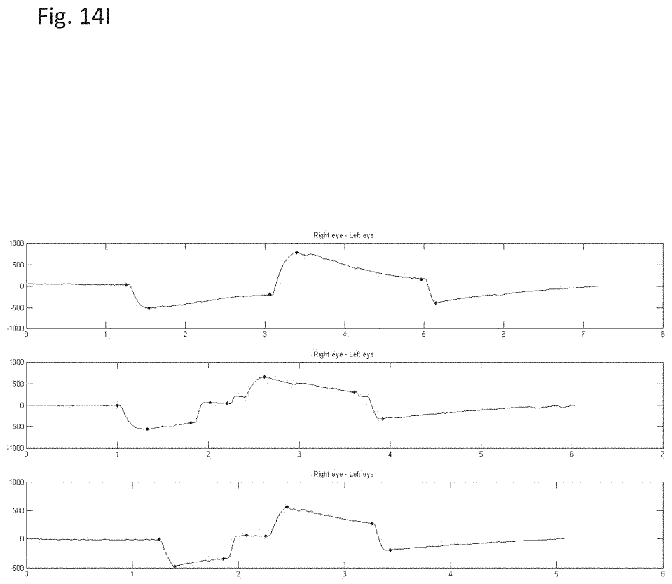

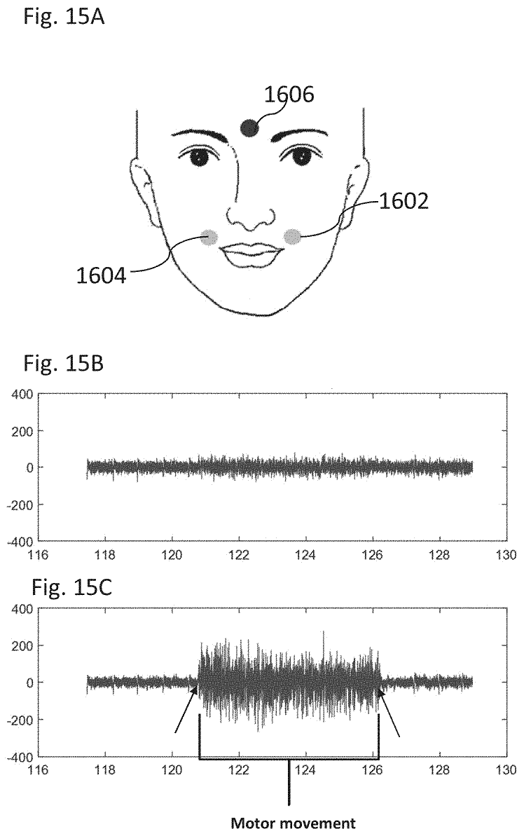

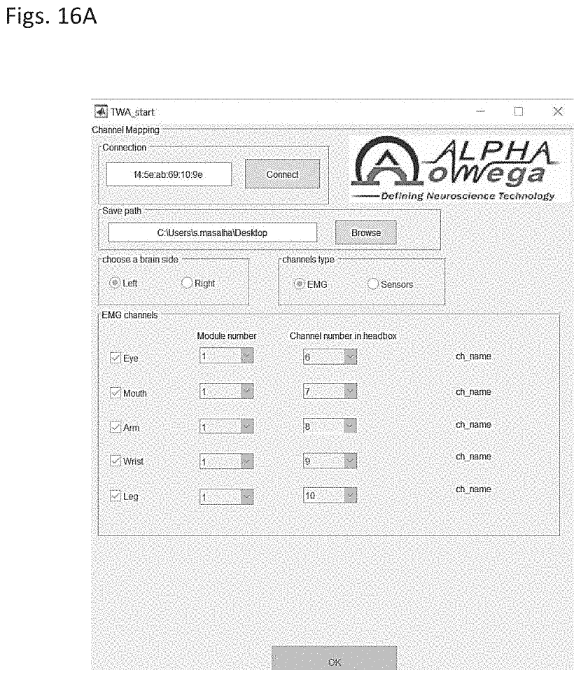

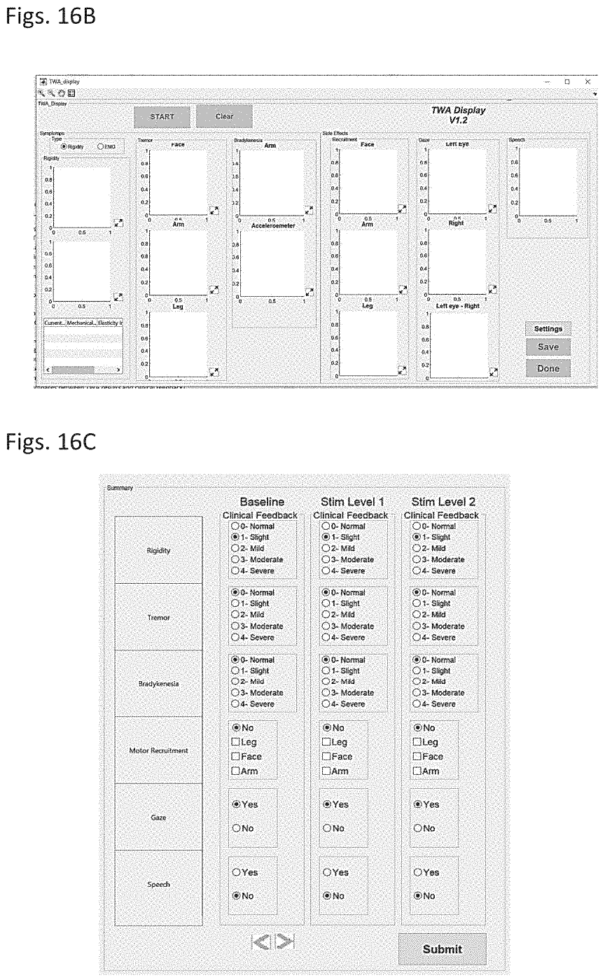

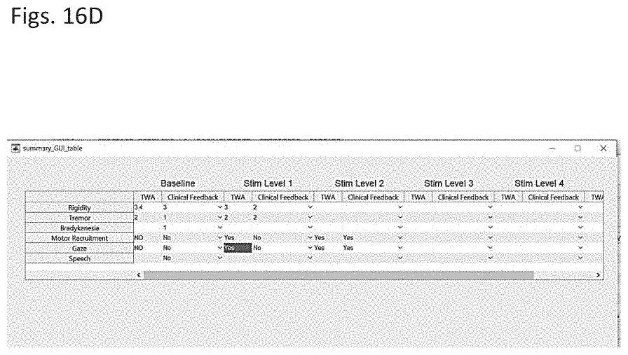

According to some exemplary embodiments, the algorithm is based on EMG signals recorded from the side of the mouth (left or right) on Zygomaticus muscles and a reference electrode on the middle of the forehead, for example as shown in FIG. 16A. FIG. 15A shows positioning of EMG Electrodes superio-lateral to the corners of the mouth of the subject, for example over left and right Zygomaticus muscles at locations. In addition, a reference electrode is positioned on the body or the face, for example over frontal bone just above the Nasion 1606.

According to some exemplary embodiments, the assessment and analysis method using the algorithm includes the following steps.

According to some exemplary embodiments, data is recorded prior to stimulation, for example in a time period of up to 10 minutes prior to the stimulation or any shorter or longer time period, and during stimulation. In some embodiments, a Low-pass filter, e.g. Butter worth filter 3 poles is applied on the signal, for example to remove stimulation artifact between 2-100 Hz. In some embodiments, the average and standard deviation (STD) of the difference in the data recorded before stimulation are calculated. Additionally or alternatively, the median and median absolute deviation (MAD) or similar indices of centrality and variability of the data are calculated. In some embodiments, a point when the difference (of the data during stimulation) reaches higher than the average+3 times the STD (or another threshold defining substantial deviation from the “center” of the data, e.g. in terms of median and MAD) is identified, for example to detect start of moving and when it decreases back.

FIG. 15B describes results of the analysis on a left mouth channel. FIG. 15C describes results of a right mouth channel, where motor movements are detected, the two arrows indicate two points detected by the algorithm.

### Exemplary Signal Pre-Processing

According to some exemplary embodiments, pre-processing of a signal acquired by at least one sensor comprises one or more of mean subtraction, normalization or standardization, analysis to components via principal component analysis (PCA) or independent component analysis (ICA), or filtering according to frequency-domain characteristics, using fixed or adaptive filters. In some embodiments, one objective of the pre-processing is to detect the moment in which DBS was administered, or configuration was changed. In some embodiments, this is done by identifying stimulation artifacts in the signal, which optionally result from electromagnetic interference between the field associated with the stimulation current and the recorded signals, characterized by spikes in the frequency domain with many high-order harmonics. In some embodiments, another objective is to minimize the effect of stimulation artifacts so that further processing can be applied to the clean signal. In some embodiments, this is accomplished by filtering, fixed or adaptive, or by a process of pattern recognition. In the latter, the repetitions of the artifact appearance are identified, a prototype artifact is constructed from this ensemble of signals, and then the prototype is subtracted from the signal at each time point in which the artifact is identified. In some embodiments, other objectives are to accentuate or attenuate specific features in the signal, such as the rhythmic low-frequency (for example in a range of 4-6 Hz, or any smaller or larger range of values) oscillations related to tremor, and/or to standardize the amplitudes such that features extracted from different subjects will be comparable.

### Exemplary Feature Construction

According to some exemplary embodiments, one way to construct signal features is to use knowledge and intuition about the signals being recorded and the attributes that are wished to be quantified. In some embodiments, signal features are constructed from one or more of fundamental tremor frequency ft, defined as the frequency with the highest power density in a band [fa, fb], in which fa≤ft≤fb, during rest; movement frequency fm, which is the frequency in which the power density increases the most when switching from rest to active movement, optionally calculated over a raw signal, a filtered signal or a low-frequency modulation envelope calculated from the signal; power density at the fundamental tremor frequency ft, normalized to the power in a frequency band [f0, f1], in which f0≤ft≤f1; total harmonic distortion (THD) relative to the fundamental tremor frequency; total power in frequencies above fhi (fhi=15, 20 or 25 Hz); highest frequency in which power density is >5% of maximum power density; correlation over time between power in frequencies above fhi and below fhi; correlation over time between pairs of signals, or signal envelopes, recorded from the various muscles; maximal cross-correlation values between pairs of signals, or signal envelopes, recorded from the various muscles; time lags respective of maximal cross-correlation values calculated for pairs of signals, or signal envelopes, recorded from the various muscles; the lags between application of stimulus and appearance of each of the other features; and degree of non-stationarity of the features—how much does the mean and variance of each feature change over time.

For example, in some embodiments the expected is parkinsonian tremor to be associated with a fundamental frequency of 4-5 Hz, high power density at ft, high THD relative to ft, high correlation between power in high-frequency (>20 Hz) and low-frequency bands and high cross correlation between limbs. In some embodiments, the expected is dyskinesia to be associated with low power density at ft, high power in frequencies above fhi, large non-stationarity and low correlation and cross-correlation between limbs. In some embodiments, rigidity is expected to be associated with high power in frequencies above fhi, low correlation between high-frequency and low-frequency bands, low non-stationarity and high cross correlation between limbs. In some embodiments, the tremor, dyskinesia and rigidity symptoms are most evident when the patient is at rest, as they manifest spontaneously and without relation to intentional movement.

In contrast, in some embodiments, bradykinesia is evident when the patient performs a motor task. In some embodiments, a feature quantifying bradykinesia comprises the envelope frequency to which most power density is added when comparing signals recorded during a repetitive movement task with signals recorded during rest. In some embodiments, the more the bradykinesia is severe, the lower the frequency of movement is expected to be.

According to some exemplary embodiments, DBS-Induced motor recruitment side effect is the result of current reaching and activating corticospinal or corticobulbar tracts of the internal capsule, leading to lower motor neuron activation and muscle contraction in the limbs or face. In some embodiments, these contractions are recorded in the EMG of the activated muscle, and the characterizing features in the EMG signal are high temporal correlation with the stimulation onset and offset, and an increase in the EMG signal amplitude and energy as the stimulation level is increased. According to some exemplary embodiments, the relation of increase in the EMG signal with larger DBS levels, is in contrast to the relation between the DBS level and EMG signal related with tremor or rigidity, which generally decreases with increasing DBS levels. Additionally, when the stimulating electrode is placed inside the target nucleus, usually some reduction of symptoms is evident before side effects, including motor recruitment, take place. In some embodiments, this sequential structure is used to differentiate between EMG signals related to the symptoms, such as rigidity, and the side-effect induced EMG.

According to some exemplary embodiments, the abovementioned expectations are used to define and construct the signal features, and the calculation of each attribute's index from the features, is statistically inferred from a database that includes 1, 2, . . . , M features calculated for each of N subjects, and optionally the respective assessment of a specialist for each of the attributes.

According to some exemplary embodiments, another way to construct the features is to generate a large library of features, regardless of intuition or prior knowledge. In some embodiments, a database with features and specialist assessments of attributes is used to statistically highlight the features that have a high predictive value for the various attributes. Examples of library features include projections of signals on PCA principal components, powers in frequency bands—e.g. in the bands [1-5 Hz], [5-10 Hz], [15-20 Hz] etc., distance between 1^{st} quartile and 3^{rd} quartile of signal amplitude.

### Exemplary Fixed and Adaptive Filtering

According to some exemplary embodiments, fixed filtering is applying a predefined filter on the data, while adaptive filtering means that the filter is dependent on the signal characteristics. For example, in order to obtain a signal that is relatively clean of the low frequency component of tremor, a fixed 4- or 8-pole Butterworth IIR high-pass filter with cut-off frequency f_{c}=20 Hz, or a fixed high order (e.g. N=2345) FIR high-pass filter with cut-off frequency fc=20 Hz are used. In some embodiments, adaptive filtering aimed at the same goal begins in performing spectral density analysis on the input signal and locating the fundamental frequency fₜ according to the highest power density between 2-6 Hz. Following this stage, an IIR or FIR filter is designed to have a cut-off frequency of 5fₜ, thus filtering out the 1^{st} 5 harmonics of fₜ.

### Exemplary Display

According to some exemplary embodiments, the assessment system comprises a display, for example a therapeutic space assessment (TSA) display. In some embodiments, The Display software (SW) is a user interface which connects to a DBS, optionally by wireless transmission. In some embodiments, the display SW collects data online from the system and run the different analysis functions to assess patient condition, for example assessment of Rigidity, Tremor, Bradykinesia, Motor recruitment, Gaze and speech. In some embodiments, the SW presents and saves the results numerically and/or graphically. In some embodiments, the SW has settings window available for the user to edit the software settings. In some embodiments, the SW enables the user to log his clinical feedback, or to insert any other data into the software, including assessments of symptoms and side effects that are not quantified by the system. In some embodiments, the SW presents a summary table comparing the TSA results with the clinical feedback, and/or ranking or scoring of different measurements.

According to some exemplary embodiments, the SW is divided by four main windows:
1. A startup window, for example as shown in FIG. 16A, which allows, for example, connection to the DBS system, and/or defining EMG and sensor channel mapping.
2. A TSA Display, for example as shown in FIG. 16B, which includes at least one display adaptor, a setting window, and a save button. Additionally, the TSA display includes a user interface for allowing access to a clinical feedback input window.
3. A clinical feedback input user interface, for example as shown in FIG. 16C.
4. A summary table that allows, for example, to compare between TSA results and clinical feedback, for example as shown in FIG. 16D.

It is expected that during the life of a patent maturing from this application many relevant DBS systems will be developed; the scope of the term DBS system is intended to include all such new technologies a priori. As used herein with reference to quantity or value, the term “about” means “within ±10% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. A method for selecting stimulation treatment parameter values, comprising:
receiving signals related to a patient condition from at least one sensor, during and/or following at least one brain stimulation session, in which stimulation is delivered in at least one location within the brain, using at least one set of treatment parameter values;
analyzing said received signals;
quantitatively assessing at least one treatment side effect and at least one symptomatic effect based on the analyzed received signals;
selecting a set of treatment parameter values based on said quantitative assessment of said treatment side effects and said symptomatic effect.

2. A method according to claim 1, comprising mapping a therapeutic space based on results of said quantitative assessment of said at least one treatment side effect and said at least one symptomatic effect, wherein said therapeutic space is a multi-dimensional space defined by two or more treatment parameter values that promote a desired therapeutic effect with a desired level of side effects.

3. A method according to claim 1, wherein said analyzing comprises analyzing said received signals following and/or during an implantation surgery.

4. (canceled)

5. A method according to claim 1, wherein said analyzing comprises analyzing said received signals using one or more statistical methods to said quantitatively assess said at least one treatment side effect and at least one symptomatic effect.

6. A method according to claim 1, comprising recording said received signals when the patient is at rest and/or when a patient performs a task.

7. A method according to claim 1, wherein said selecting comprises selecting said set of treatment parameter values based on future flexibility of said selected set of treatment parameter values.

8-10. (canceled)

11. A method according to claim 7, wherein said future flexibility is based on future changes in an at least one therapeutic effect modifier capable of affecting therapy and/or tuning ability in the future.

12-18. (canceled)

19. A method according to claim 1, wherein said quantitatively assessing comprises quantitatively assessing one or more of gaze deviation and diplopia, continuous activation of muscles in legs, arms or face, dyskinesia, muscle rigidity, tremor and bradykinesia.

20. A method according to claim 1, wherein said selecting comprises selecting a set of values related to stimulation amplitude, stimulation frequency and/or stimulation duration of a stimulation treatment.

21. (canceled)

22. A method for mapping therapeutic space, comprising:
receiving signals related to a patient condition from at least one sensor, during and/or following at least one brain stimulation delivered in at least one location within the brain, using at least one set of treatment parameter values;
analyzing said received signals;
quantitatively assessing at least one treatment side effect and at least one symptomatic effect based on said analyzed received signals;
mapping therapeutic space based on said quantitative assessment.

23. A method according to claim 22, wherein said mapping comprises mapping said therapeutic space based on a desired future flexibility.

24. A method according to claim 22, comprising recording said signals when the patient is at rest or when a patient performs a task.

25. A method according to claim 22, comprising determining that a stimulation electrode or an electrode lead is positioned in a correct location inside the brain.

26. A method according to claim 22, comprising selecting at least one set of treatment parameter values based on said mapping of said therapeutic space.

27. (canceled)

28. A system for selecting a set of treatment parameter values for a brain stimulation treatment, comprising:
a control circuitry;
a memory connected to said control circuitry, wherein said memory stores signals related to a patient condition continuously measured during and/or following at least one brain stimulation, at least one set of treatment parameter values used for said brain stimulation;
an analysis circuitry connected to said control circuitry, wherein said control circuitry signals said analysis circuitry to quantitatively assess at least one treatment side effect and at least one symptomatic effect of said brain stimulation based on said stored continuously measured signals;
a user interface connected to said control circuitry, wherein said user interface is configured to deliver an indication regarding said at least one treatment side effect and said at least one symptomatic effect.

29. A system according to claim 28, wherein said control circuitry generates a map of a therapeutic space based on said quantitative assessment of said at least one treatment side effect and at least one symptomatic effect, and signals said user interface to deliver an indication regarding said mapped therapeutic space, wherein said therapeutic space is a multi-dimensional space defined by two or more treatment parameter values that promote a desired therapeutic effect with a desired level of side effects.

30. A system according to claim 29, wherein said control circuitry calculates at least one optional set of treatment parameter values based on said mapped therapeutic space.

31. (canceled)

32. A system according to claim 29, wherein said control circuitry calculates a relation between at least one set of treatment parameter values and said mapped therapeutic space, and signals said user interface to deliver an indication regarding said relation.

33. (canceled)

34. A system according to claim 28, wherein said analysis circuitry calculates said at least one value of a future flexibility based on said quantitative assessment of said at least one treatment side effect and said at least one symptomatic effect and/or said at least one set of treatment parameter values stored in said memory.

35-42. (canceled)

43. A system according to claim 28, wherein said at least one treatment side effect comprises gaze deviation, diplopia, continuous activation of muscles in legs, arms or face, and dyskinesia.

44-48. (canceled)
